(19) United States
(12) Patent Application Publication
(10) Pub. No.: US 2007/0117797 A1
(43) Pub. Date: May 24, 2007

(54) Alkylamino, arylamino, and sulfonamido cyclopentyl amide modulators of chemokine receptor activity

(76) Inventors: Stephen D. Goble, Edison, NJ (US); Lihu Yang, Edison, NJ (US); Changyou Zhou, Plainsboro, NJ (US); Shankaran Kothandaraman, Kendall Park, NJ (US); Deodialsingh Guiadeen, Linden, NJ (US); Gabor Butora, Martinsville, NJ (US); Alexander Pastemak, Princeton, NJ (US); Sander G. Mills, Scotch Plains, NJ (US)

Correspondence Address: MERCK AND CO., INC, P O BOX 2000, RAHWAY, NJ, 07065-0907, US

(21) Appl. No.: 10/585,232

(22) PCT Filed: Dec. 29, 2004
(86) PCT No.: PCT/US04/43777

§ 371 Date: Jun. 30, 2006

Related U.S. Application Data
(60) Provisional application No. 60/533,892, filed on Jan. 2, 2004.

(51) Int. Cl. A61K 31/538; A61K 31/4745; A61K 31/47; A61K 31/426; A61K 31/16

(52) U.S. Cl. 514/230.5; 546/122; 546/146; 546/340; 544/90; 548/190; 514/300; 514/310; 514/365; 514/616

(57) ABSTRACT

Compounds of the formula (I) which are modulators of chemokine receptor activity useful in the prevention or treatment of certain inflammatory and immunoregulatory disorders and diseases, allergic diseases, atopic conditions including allergic rhinitis, dermatitis, conjunctivitis, and asthma, as well as autoimmune pathologies such as rheumatoid arthritis and atherosclerosis, and pharmaceutical compositions comprising these compounds and the use of these compounds and compositions in the prevention or treatment of such diseases in which chemokine receptors are involved.

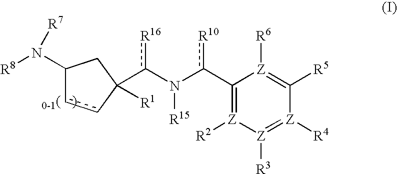

### BACKGROUND OF THE INVENTION

The chemokines are a family of small (70-120 amino acids), proinflammatory cytokines, with potent chemotactic activities. Chemokines are chemotactic cytokines that are released by a wide variety of cells to attract various cells, such as monocytes, macrophages, T cells, eosinophils, basophils and neutrophils to sites of inflammation (reviewed in Schall, Cytokine, 3, 165-183 (1991) and Murphy, Rev. Immun., 12, 593-633 (1994)). These molecules were originally defined by four conserved cysteines and divided into two subfamilies based on the arrangement of the first cysteine pair. In the CXC-chemokine family, which includes IL-8, GROα, NAP-2 and IP-10, these two cysteines are separated by a single amino acid, while in the CC-chemokine family, which includes RANTES, MCP-1, MCP-2, MCP-3, MIP-1α, MIP-1β and eotaxin, these two residues are adjacent.

The α-chemokines, such as interleukin-8 (IL-8), neutrophil-activating protein-2 (NAP-2) and melanoma growth stimulatory activity protein (MGSA) are chemotactic primarily for neutrophils, whereas β-chemokines, such as RANTES, MIP-1α, MIP-1β, monocyte chemotactic protein-1 (MCP-1), MCP-2, MCP-3 and eotaxin are chemotactic for macrophages, monocytes, T-cells, eosinophils and basophils (Deng, et al., Nature, 381, 661-666 (1996)).

The chemokines are secreted by a wide variety of cell types and bind to specific G-protein coupled receptors (GPCRs) (reviewed in Horuk, Trends Pharm. Sci., 15, 159-165 (1994)) present on leukocytes and other cells. These chemokine receptors form a sub-family of GPCRs, which, at present, consists of fifteen characterized members and a number of orphans. Unlike receptors for promiscuous chemoattractants such as C5a, fMLP, PAF, and LTB4, chemokine receptors are more selectively expressed on subsets of leukocytes. Thus, generation of specific chemokines provides a mechanism for recruitment of particular leukocyte subsets.

On binding their cognate ligands, chemokine receptors transduce an intracellular signal though the associated trimeric G protein, resulting in a rapid increase in intracellular calcium concentration. There are at least seven human chemokine receptors that bind or respond to β-chemokines with the following characteristic pattern: CCR-1 (or “CKR-1” or “CC—CKR-1”) [MIP-1α, MIP-1β, MCP-3, RANTES] (Ben-Barruch, et al., J. Biol. Chem., 270, 22123-22128 (1995); Beote, et al, Cell, 72, 415-425 (1993)); CCR-2A and CCR-2B (or “CKR-2A”/“CKR-2A” or “CC—CKR-2A”/“CC—CKR-2A”) [MCP-1, MCP-2, MCP-3, MCP-4]; CCR-3 (or “CKR-3” or “CC—CKR-3”) [Eotaxin, Eotaxin 2, RANTES, MCP-2, MCP-3] (Rollins, et al., Blood, 90, 908-928 (1997)); CCR-4 (or “CKR-4” or “CC—CKR-4”) [MIP-1α, RANTES, MCP-1] (Rollins, et al., Blood, 90, 908-928 (1997)); CCR-5 (or “CKR-5” or “CC—CKR-5”) [MIP-1α, RANTES, MIP-1β] (Sanson, et al., Biochemistry, 35, 3362-3367 (1996)); and the Duffy blood-group antigen [RANTES, MCP-1] (Chaudhun, et al., J. Biol. Chem., 269, 7835-7838 (1994)). The β-chemokines include eotaxin, MIP (“macrophage inflammatory protein”), MCP (“monocyte chemoattractant protein”) and RANTES (“regulation-upon-activation, normal T expressed and secreted”) among other chemokines.

Chemokine receptors, such as CCR-1, CCR-2, CCR-2A, CCR-2B, CCR-3, CCR-4, CCR-5, CXCR-3, CXCR-4, have been implicated as being important mediators of inflammatory and immunoregulatory disorders and diseases, including asthma, rhinitis and allergic diseases, as well as autoimmune pathologies such as rheumatoid arthritis and atherosclerosis. Humans who are homozygous for the 32-basepair deletion in the CCR-5 gene appear to have less susceptibility to rheumatoid arthritis (Gomez, et al., Arthritis & Rheumatism, 42, 989-992 (1999)). A review of the role of eosinophils in allergic inflammation is provided by Kita, H., et al., J. Exp. Med. 183, 2421-2426 (1996). A general review of the role of chemokines in allergic inflammation is provided by Lustger, A. D., New England J. Med., 338(7), 426-445 (1998).

A subset of chemokines are potent chemoattractants for monocytes and macrophages. The best characterized of these is MCP-1 (monocyte chemoattractant protein-1), whose primary receptor is CCR2. MCP-1 is produced in a variety of cell types in response to inflammatory stimuli in various species, including rodents and humans, and stimulates chemotaxis in monocytes and a subset of lymphocytes. In particular, MCP-1 production correlates with monocyte and macrophage infiltration at inflammatory sites. Deletion of either MCP-1 or CCR2 by homologous recombination in mice results in marked attenuation of monocyte recruitment in response to thioglycollate injection and Listeria monocytogenes infection (Lu et al., J. Exp. Med., 187, 601-608 (1998); Kurihara et al. J. Exp. Med., 186, 1757-1762 (1997); Boring et al. J. Clin. Invest., 100, 2552-2561 (1997); Kuziel et al. Proc. Natl. Acad. Sci., 94, 12053-12058 (1997)). Furthermore, these animals show reduced monocyte infiltration into granulomatous lesions induced by the injection of schistosomal or mycobacterial antigens (Boring et al. J. Clin. Invest., 100, 2552-2561 (1997); Warmington et al. Am J. Path., 154, 1407-1416 (1999)). These data suggest that MCP-1-induced CCR2 activation plays a major role in monocyte recruitment to inflammatory sites, and that antagonism of this activity will produce a sufficient suppression of the immune response to produce therapeutic benefits in immunoinflammatory and autoimmune diseases.

Accordingly, agents which modulate chemokine receptors such as the CCR-2 receptor would be useful in such disorders and diseases.

In addition, the recruitment of monocytes to inflammatory lesions in the vascular wall is a major component of the pathogenesis of atherogenic plaque formation. MCP-1 is produced and secreted by endothelial cells and intimal smooth muscle cells after injury to the vascular wall in hypercholesterolemic conditions. Monocytes recruited to the site of injury infiltrate the vascular wall and differentiate to foam cells in response to the released MCP-1. Several groups have now demonstrated that aortic lesion size, macrophage content and necrosis are attenuated in MCP-1 −/− or CCR2 −/− mice backcrossed to APO-E −/−, LDL-R −/− or Apo B transgenic mice maintained on high fat diets (Boring et al. Nature, 394, 894-897 (1998); Gosling et al. J. Clin. Invest., 103, 773-778 (1999)). Thus, CCR2 antagonists may inhibit atherosclerotic lesion formation and pathological progression by impairing monocyte recruitment and differentiation in the arterial wall.

### SUMMARY OF THE INVENTION

The present invention is directed to compounds of the formula: 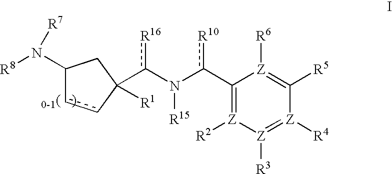
which are modulators of chemokine receptor activity and are useful in the prevention or treatment of certain inflammatory and immunoregulatory disorders and diseases, allergic diseases, atopic conditions including allergic rhinitis, dermatitis, conjunctivitis, and asthma, as well as autoimmune pathologies such as rheumatoid arthritis and atherosclerosis. The invention is also directed to pharmaceutical compositions comprising these compounds and the use of these compounds and compositions in the prevention or treatment of such diseases in which chemokine receptors are involved.

### DETAILED DESCRIPTION OF THE INVENTION

The present invention is directed to compounds of the formula I: 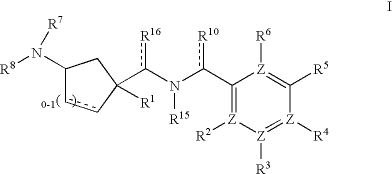
wherein:
Z is N or C, where no more than two Z are N;
R¹ is selected from: —C₁₋₆alkyl, —C₀₋₆alkyl-O—C₁₋₆alkyl, —C₀₋₆alkyl-S—C₁₋₆alkyl, —C₀₋₆alkyl-SO₂—C₁₋₆alkyl, —C₀₋₆alkyl-SO—C₁₋₆alkyl, —C₀₋₆alkyl-SO₂—NR¹²—C₀₋₆alkyl, —(C₀₋₆alkyl)-(C₃₋₇cycloalkyl)-(C₀₋₆alkyl), hydroxy, heterocycle, —CN, —NR¹²R¹², —NR¹²COR¹³, —NR¹²SO₂R¹⁴, —COR¹¹, —CONR¹²R¹², and phenyl;, where alkyl and the cycloalkyl are unsubstituted or substituted with 1-7 substituents independently selected from: halo, hydroxy, —O—C₁₋₃alkyl, trifluoromethyl, C₁₋₃alkyl, —O—C₁₋₃alkyl, —COR¹¹, —SO₂R¹⁴, —NHCOR¹⁵, —NHSO₂CH₃, —heterocycle, ═O, and —CN, and where phenyl and heterocycle are independently unsubstituted or substituted with 1-3 substituents independently selected from: halo, hydroxy, C₁₋₃alkyl, C₁₋₃alkoxy, trifluoromethyl and NHCOR¹⁵;
when the Z attached to R² is N, R² is oxygen or is absent, and when the Z attached to R² is C, R² is selected from: hydrogen, C₁₋₃alkyl optionally substituted with 1-3 fluoro, —O—C₁₋₃alkyl optionally substituted with 1-3 fluoro, hydroxy, chloro, fluoro, bromo and phenyl;
when the Z attached to R³ is N, R³ is oxygen or is absent, and when the Z attached to R³ is C, R³ is selected from: hydrogen, hydroxy, halo, C₁₋₃alkyl where the alkyl is unsubstituted or substituted with 1-6 substituents independently selected from: fluoro, hydroxy and —COR¹¹, —NR¹²R¹², —COR¹¹, —CONR¹²R¹², —NR¹²COR¹³, —OCONR¹²R¹², —NR¹²CONR¹²R¹², —heterocycle, —CN, —NR¹²—SO₂—NR¹²R¹², —NR¹²—SO₂—R¹⁴, —SO₂—NR¹²R¹² and nitro;
when the Z attached to R⁴ is N, R⁴ is oxygen or is absent, and when the Z attached to R⁴ is C, R⁴ is selected from: hydrogen, C₁₋₃alkyl optionally substituted with 1-3 fluoro, —O—C₁₋₃alkyl optionally substituted with 1-3 fluoro, hydroxy, chloro, fluoro, bromo and phenyl;
R⁵ is selected from: C₁₋₆alkyl where alkyl is unsubstituted or substituted with 1-6 substituents selected from fluoro and hydroxyl, —O—C₁₋₆alkyl where alkyl is unsubstituted or substituted with 1-6 fluoro, —CO—C₁₋₆alkyl where alkyl is unsubstituted or substituted with 1-6 fluoro, —S—C₁₋₆alkyl where alkyl is unsubstituted or substituted with 1-6 fluoro, pyridyl which is unsubstituted or substituted with one or more substituents selected from: halo, trifluoromethyl, C₁₋₄alkyl, and COR¹¹, fluoro, chloro, bromo, —C₄₋₆cycloalkyl, —O—C₄₋₆cycloalkyl, phenyl which is unsubstituted or substituted with one or more substituents selected from halo, trifluoromethyl, C₁₋₄alkyl, and COR¹¹, —O-phenyl which is unsubstituted or substituted with one or more substituents selected from: halo, trifluoromethyl, C₁₋₄alkyl, and COR¹¹, —C₃₋₆cycloalkyl where alkyl is unsubstituted or substituted with 1-6 fluoro, —O—C₃₋₆cycloalkyl where alkyl is unsubstituted or substituted with 1-6 fluoro, -heterocycle, —CN and —COR¹¹;
when the Z attached to R⁶ is N, R⁶ is oxygen or is absent, and when the Z attached to R⁶ is C, R⁶ is selected from: hydrogen, C₁₋₃alkyl optionally substituted with 1-3 fluoro, —O—C₁₋₃alkyl optionally substituted with 1-3 fluoro, hydroxy, chloro, fluoro, bromo and phenyl;
R⁷ is selected from: hydrogen, C₁₋₈alkyl which is unsubstituted or substituted with 1-6 substituents selected from: hydroxy, halo, —O—C₁₋₆alkyl, CN, —NR¹²R¹², —NR¹²COR¹³, —NR¹²SO₂R¹⁴, —COR¹¹, —CONR¹²R¹², phenyl and heterocycle, where the alkyl, phenyl, and heterocycle are unsubstituted or substituted with 1-3 substituents selected from: halo, hydroxy, C₁₋₃alkyl, C₁₋₃alkoxy, —CO₂H, —CO₂—C₁₋₆alkyl, and trifluoromethyl, and —SO₂C₁₋₆alkyl which is unsubstituted or substituted with 1-6 substituents selected from: hydroxy, halo, —O—C₁₋₆alkyl, CN, —NR¹²R¹², —NR¹²COR¹³, —NR¹²SO₂R¹⁴, —COR¹¹, —CONR¹²R¹², phenyl and heterocycle, where the alkyl, phenyl, and heterocycle are unsubstituted or substituted with 1-3 substituents selected from: halo, hydroxy, C₁₋₃alkyl, C₁₋₃alkoxy, —CO₂H, —CO₂—C₁₋₆alkyl, and trifluoromethyl;
R⁸ is selected from C₁₋₁₀alkyl, —SO₂C₁₋₁₀alkyl, pyridyl or phenyl, unsubstituted or substituted with 1-5 substituents selected from: hydroxy, halo, —O—C₁₋₆alkyl, —S—C₁₋₆alkyl, CN, —NR¹²R¹², —NR¹²COR¹³, —NR¹²SO₂R¹⁴, —COR¹¹, —CONR¹²R¹², —SO₂R¹⁴, heterocycle, ═O (where the oxygen is connected via a double bond), phenoxy and phenyl, where the alkyl, phenyl, phenoxy and heterocycle are unsubstituted or substituted with 1-3 substituents selected from: halo, hydroxy, C₁₋₃alkyl, C₁₋₃alkoxy, —COR¹¹, —CN, —NR¹²R¹², —SO₂R¹⁴, —NR¹²COR¹³, —NR¹²SO₂R¹⁴ and —CONR¹²R¹², where the alkyl and alkoxy are optionally substituted with 1-5 fluoro;
R¹⁰ and R¹⁶ are independently selected from: ═O, hydrogen, phenyl, C₁₋₆alkyl which is unsubstituted or substituted with 1-6 of the following substituents: —COR¹¹, hydroxy, fluoro, chloro, and —O—C₁₋₃alkyl; and,
R¹¹ is independently selected from: hydroxy, hydrogen, C₁₋₆ alkyl, —O—C₁₋₆alkyl, benzyl, phenyl, C₃₋₆ cycloalkyl, where the alkyl, phenyl, benzyl, and cycloalkyl groups are unsubstituted or substituted with 1-3 substituents independently selected from: halo, hydroxy, C₁₋₃alkyl, C₁₋₃alkoxy, —CO₂H, —CO₂—C₁₋₆ alkyl, and trifluoromethyl,
R¹² is selected from: hydrogen, C₁₋₆ alkyl, benzyl, phenyl, C₃₋₆ cycloalkyl, where the alkyl, phenyl, benzyl, and cycloalkyl groups are unsubstituted or substituted with 1-3 substituents independently selected from: halo, hydroxy, C₁₋₃alkyl, C₁₋₃alkoxy, —CO₂H, —CO₂—C₁₋₆alkyl, and trifluoromethyl, and
R¹³ is selected from: hydrogen, C₁₋₆ alkyl, —O—C₁₋₆alkyl, benzyl, phenyl, C₃₋₆ cycloalkyl, where the alkyl, phenyl, benzyl, and cycloalkyl groups are unsubstituted or substituted with 1-3 substituents independently selected from: halo, hydroxy, C₁₋₃alkyl, C₁₋₃alkoxy, —CO₂H, —CO₂—C₁₋₆alkyl, and trifluoromethyl,
R¹⁴ is selected from: hydroxy, C₁₋₆ alkyl, —O—C₁₋₆alkyl, benzyl, phenyl, C₃₋₆ cycloalkyl, where the alkyl, phenyl, benzyl, and cycloalkyl groups are unsubstituted or substituted with 1-3 substituents independently selected from: halo, hydroxy, C₁₋₃alkyl, C₁₋₃alkoxy, —CO₂H, —CO₂—C₁₋₆alkyl, and trifluoromethyl,
R¹⁵ is selected from hydrogen and C1-3alkyl;
or, R² and R¹⁵ are joined together to form a carbocycle or heterocycle ring with a linker selected from: —CH₂(CR¹⁷R¹⁷)₁₋₃—, —CH₂NR¹⁸—, —NR¹⁸—CR¹⁷R¹⁷—, —CR¹⁷R¹⁷O—, —CR¹⁷R¹⁷SO₂—, —CR¹⁷R¹⁷SO—, —CR¹⁷R¹⁷S—, —CR¹⁷R¹⁷—, and —NR¹⁸— (with the left side of the linker being bonded to the amide nitrogen at R¹⁵),
R¹⁷ is selected from: hydrogen, hydroxy, halo and C₁₋₃alkyl, where the alkyl is unsubstituted or substituted with 1-6 substituents independently selected from: fluoro, and hydroxy, —NR¹²R¹², —COR¹¹, —CONR¹²R¹², —NR¹²COR¹³, —OCONR¹²R¹², —NR¹²CONR¹²R¹², -heterocycle, —CN, —NR¹²—SO₂—NR¹²R¹², —NR¹²—SO₂—R¹⁴, —SO₂—NR¹²R¹², and ═O, and where when one R¹⁷ is connected to the ring via a double bond the other R¹⁷ at the same position is absent,
R¹⁸ is selected from: hydrogen, C₁₋₃alkyl unsubstituted or substituted with 1-6 substituents independently selected from: fluoro, and hydroxy, COR¹³, SO₂R¹⁴, and SO₂NR¹²R¹²;
the dashed line represents an optional bond.

Preferred compounds of the present invention include compounds of formula Ia: 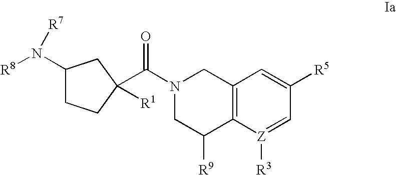
wherein R¹, R³, R⁵, R⁷, R⁸, and Z are described herein and, wherein R⁹ is selected from: hydrogen, hydroxy, C₁₋₃alkyl unsubstituted or substituted with 1-6 substituents independently selected from fluoro and hydroxy, —COR¹¹, —CONR¹²R¹², —NR¹²COR¹¹, —NR¹²—SO₂—R¹⁴, —SO₂—NR¹²R¹², and ═O, where R⁹ is connected to the ring via a double bond.

Preferred compounds of the present invention also include compounds of formula Ib: 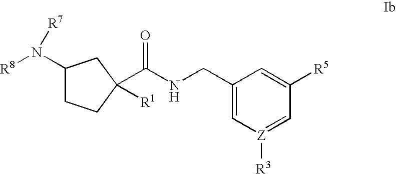
wherein R¹, R³, R⁵, R⁷, R⁸, and Z are described herein.

Preferred compounds of the present invention also include compounds of formula Ic: 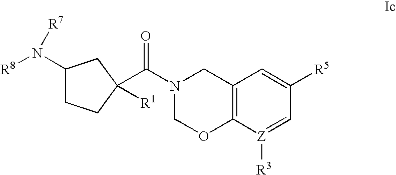
wherein R¹, R³, R⁵, R⁷, R⁸, and Z are described herein.

More preferred compounds of the present invention include compounds of formula Id: 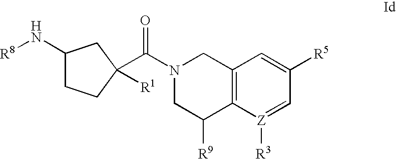
wherein R¹, R³, R⁵, R⁸, R⁹, and Z are described herein.

More preferred compounds of the present invention also include compounds of formula Ie: 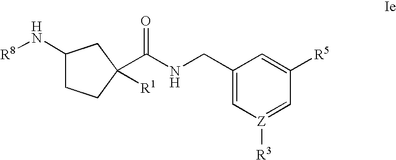
wherein R¹, R³, R⁵, R⁸, and Z are described herein.

More preferred compounds of the present invention also include compounds of formula If: 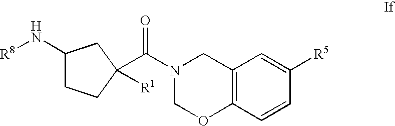
wherein R¹, R⁵, and R⁸ are described herein.

In the present invention it is preferred that R¹ is selected from: —C₁₋₆alkyl, —C₀₋₆alkyl-O—C₁₋₆alkyl, and —(C₀₋₆alkyl)-(C₃₋₇cycloalkyl)-(C₀₋₆alkyl), where the alkyl and the cycloalkyl are unsubstituted or substituted with 1-7 substituents independently selected from: halo, hydroxy, —O—C₁₋₃alkyl, trifluoromethyl, C₁₋₃alkyl, —O—C₁₋₃alkyl, —COR¹¹, —CN, —NR¹²R¹², and —CONR¹²R¹².

In the present invention it is more preferred that R¹ is selected from:
—C₁₋₆alkyl unsubstituted or substituted with 1-6 substituents independently selected from: halo, hydroxy, —O—C₁₋₃alkyl, trifluoromethyl, and —COR¹¹,
thiazolyl unsubstituted or substituted with NHCOR¹⁵,
—C₀₋₆alkyl-O—C₁₋₆alkyl-unsubstituted or substituted with 1-6 substituents independently selected from: halo, trifluoromethyl, and —COR¹¹, and
—(C₃₋₅cycloalkyl)-(C₀₋₆alkyl) unsubstituted or substituted with 1-7 substituents independently selected from: halo, hydroxy, —O—C₁₋₃alkyl, trifluoromethyl, and —COR¹¹.

In the present invention it is still more preferred that R¹ is selected from: C₁₋₆alkyl, C₁₋₆alkyl substituted with hydroxyl, and C₁₋₆alkyl substituted with 1-6 fluoro.

In the present invention it is most preferred that R¹ is selected from: —CH(CH₃)₂, —CH(OH)CH₃, and —CH₂CF₃.

In the present invention it is preferred that R² is hydrogen or R² and R¹⁵ are joined together by a tether chosed from: —CH₂—CH₂— and —CH₂—O—.

In the present invention when the Z attached to R³ is N it is preferred that R³ is nothing or O (to give a N-oxide).

In the present invention when the Z attached to R³ is N it is more preferred that R³ is nothing.

In the present invention when the Z attached to R³ is C it is preferred that R³ is selected from: hydrogen, halo, hydroxy, C₁₋₃alkyl, where the alkyl is unsubstituted or substituted with 1-6 substituents independently selected from: fluoro, and hydroxy, —COR¹¹, —CONR¹²R¹², -heterocycle, —NR¹²—SO₂—NR¹²R¹², —NR¹²—SO₂—R¹⁴, —SO₂—NR¹²R¹², -nitro, and —NR¹²R¹².

In the present invention when the Z attached to R³ is C it is more preferred that R³ is hydrogen, fluoro, or trifluoromethyl.

In the present invention it is preferred that the Z attached to R⁴ is C.

In the present invention it is preferred that R⁴ is hydrogen.

In the present invention it is preferred that R⁵ is selected from: C₁₋₆alkyl substituted with 1-6 fluoro, —O—C₁₋₆alkyl substituted with 1-6 fluoro, chloro, bromo and phenyl.

In the present invention it is more preferred that R⁵ is selected from: trifluoromethyl, trifluoromethoxy, chloro, bromo, and phenyl.

In the present invention it is most preferred that R⁵ is trifluoromethyl.

In the present invention it is preferred that the Z attached to R⁶ is C.

In the present invention it is preferred that R⁶ is hydrogen.

In the present invention it is preferred that R⁷ is hydrogen or methyl.

In the present invention it is more preferred that R⁷ is hydrogen.

In the present invention it is preferred that R⁸ is selected from the following: C₁₋₈alkyl optionally substituted with hydroxy, C₁₋₆alkyl substituted with 1-6 fluoro, C₁₋₆alkyl substituted with —COR¹¹, benzyl, unsubstituted or substituted with 1-3 substituents selected from: hydroxy, methoxy, chloro, fluoro, —COR¹¹, methyl and trifluoromethyl, —CH₂-pyridyl, unsubstituted or substituted with 1-3 substituents selected from: hydroxy, methoxy, chloro, fluoro, methyl and trifluoromethyl.

In the present invention it is preferred that R⁹ is selected from: hydroxyl, hydrogen, ═O, where R⁹ is connected to the ring via a double bond.

In the present invention it is more preferred that R⁹ is hydrogen.

In the present invention it is preferred that R¹⁰ is hydrogen.

In the present invention it is preferred that R¹⁵ is hydrogen or is joined to R² as described in R².

In the present invention it is preferred that R¹⁶ is oxygen and is connected via a double bond.

The independent syntheses of diastereomers and enantiomers or their chromatographic separations may be achieved as known in the art by appropriate modification of the methodology disclosed herein. Their absolute stereochemistry may be determined by the x-ray crystallography of crystalline products or crystalline intermediates which are derivatized, if necessary, with a reagent containing an asymmetric center of known absolute configuration.

The independent syntheses of diastereomers and enantiomers or their chromatographic separations may be achieved as known in the art by appropriate modification of the methodology disclosed herein. Their absolute stereochemistry may be determined by the x-ray crystallography of crystalline products or crystalline intermediates which are derivatized, if necessary, with a reagent containing an asymmetric center of known absolute configuration.

As appreciated by those of skill in the art, halo or halogen as used herein are intended to include chloro, fluoro, bromo and iodo. Similarly, C₁₋₈, as in C₁₋₈alkyl is defined to identify the group as having 1, 2, 3, 4, 5, 6, 7 or 8 carbons in a linear or branched arrangement, such that C₁₋₈alkyl specifically includes methyl, ethyl, n-propyl, iso-propyl, n-butyl, iso-butyl, tert-butyl, pentyl, hexyl, heptyl and octyl. Likewise, C₀, as in C₀alkyl is defined to identify the presence of a direct covalent bond. The term “heterocycle” as used herein is intended to include the following groups: benzoimidazolyl, benzofuranyl, benzofurazanyl, benzopyrazolyl, benzotriazolyl, benzothiophenyl, benzoxazolyl, carbazolyl, carbolinyl, cinnolinyl, furanyl, imidazolyl, indolinyl, indolyl, indolazinyl, indazolyl, isobenzofuranyl, isoindolyl, isoquinolyl, isothiazolyl, isoxazolyl, naphthpyridinyl, oxadiazolyl, oxazolyl, oxetanyl, pyranyl, pyrazinyl, pyrazolyl, pyridazinyl, pyridopyridinyl, pyridazinyl, pyridyl, pyrimidyl, pyrrolyl, quinazolinyl, quinolyl, quinoxalinyl, tetrahydropyranyl, tetrazolyl, tetrazolopyridyl, thiadiazolyl, thiazolyl, thienyl, triazolyl, azetidinyl, 1,4-dioxanyl, hexahydroazepinyl, piperazinyl, piperidinyl, pyrrolidinyl, morpholinyl, thiomorpholinyl, dihydrobenzoim idazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, methylenedioxybenzoyl, tetrahydrofuranyl, and tetrahydrothienyl, and N-oxides thereof.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, “pharmaceutically acceptable salts” refer to derivatives wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like.

The pharmaceutically acceptable salts of the present invention can be prepared from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media such as ether, ethyl acetate, ethanol, isopropanol, or acetonitrile are preferred. Suitable salts are found, e.g. in Remington's Pharmaceutical Sciences, 17th ed., Mack Publishing Company, Easton, Pa., 1985, p. 1418.

Exemplifying the invention is the use of the compounds disclosed in the Examples and herein.

Specific compounds within the present invention include a compound which selected from the group consisting of: the title compounds of the Examples; and pharmaceutically acceptable salts thereof and individual diastereomers thereof.

The subject compounds are useful in a method of modulating chemokine receptor activity in a patient in need of such modulation comprising the administration of an effective amount of the compound.

The present invention is directed to the use of the foregoing compounds as modulators of chemokine receptor activity. In particular, these compounds are useful as modulators of the chemokine receptors, in particular CCR-2.

The utility of the compounds in accordance with the present invention as modulators of chemokine receptor activity may be demonstrated by methodology known in the art, such as the assay for chemokine binding as disclosed by Van Riper, et al., J. Exp. Med., 177, 851-856 (1993) which may be readily adapted for measurement of CCR-2 binding.

Receptor affinity in a CCR-2 binding assay was determined by measuring inhibition of ¹²⁵I-MCP-1 to the endogenous CCR-2 receptor on various cell types including monocytes, THP-1 cells, or after heterologous expression of the cloned receptor in eukaryotic cells. The cells were suspended in binding buffer (50 mM HEPES, pH 7.2, 5 mM MgCl₂, 1 mM CaCl₂, and 0.50% BSA) with and added to test compound or DMSO and ¹²⁵I-MCP-1 at room temperature for 1 h to allow binding. The cells were then collected on GFB filters, washed with 25 mM HEPES buffer containing 500 mM NaCl and cell bound ¹²⁵I-MCP-1 was quantified.

In a chemotaxis assay chemotaxis was performed using T cell depleted PBMC isolated from venous whole or leukophoresed blood and purified by Ficoll-Hypaque centrifugation followed by rosetting with neuraminidase-treated sheep erythrocytes. Once isolated, the cells were washed with HBSS containing 0.1 mg/ml BSA and suspended at 1×10⁷ cells/ml. Cells were fluorescently labeled in the dark with 2 μM Calcien-AM (Molecular Probes), for 30 min at 37° C. Labeled cells were washed twice and suspended at 5×10⁶ cells/ml in RPMI 1640 with L-glutamine (without phenol red) containing 0.1 mg/ml BSA. MCP-1 (Peprotech) at 10 ng/ml diluted in same medium or medium alone were added to the bottom wells (27 μl). Monocytes (150,000 cells) were added to the topside of the filter (30 μl) following a 15 min preincubation with DMSO or with various concentrations of test compound. An equal concentration of test compound or DMSO was added to the bottom well to prevent dilution by diffusion. Following a 60 min incubation at 37° C., 5% CO₂, the filter was removed and the topside was washed with HBSS containing 0.1 mg/ml BSA to remove cells that had not migrated into the filter. Spontaneous migration (chemokinesis) was determined in the absence of chemoattractant

In particular, the compounds of the following examples had activity in binding to the CCR-2 receptor in the aforementioned assays, generally with an IC₅₀ of less than about 1 μM. Such a result is indicative of the intrinsic activity of the compounds in use as modulators of chemokine receptor activity.

Mammalian chemokine receptors provide a target for interfering with or promoting eosinophil and/or lymphocyte function in a mammal, such as a human. Compounds which inhibit or promote chemokine receptor function, are particularly useful for modulating eosinophil and/or lymphocyte function for therapeutic purposes. Accordingly, compounds which inhibit or promote chemokine receptor function would be useful in treating, preventing, ameliorating, controlling or reducing the risk of a wide variety of inflammatory and immunoregulatory disorders and diseases, allergic diseases, atopic conditions including allergic rhinitis, dermatitis, conjunctivitis, and asthma, as well as autoimmune pathologies such as rheumatoid arthritis and atherosclerosis.

For example, an instant compound which inhibits one or more functions of a mammalian chemokine receptor (e.g., a human chemokine receptor) may be administered to inhibit (i.e., reduce or prevent) inflammation. As a result, one or more inflammatory processes, such as leukocyte emigration, chemotaxis, exocytosis (e.g., of enzymes, histamine) or inflammatory mediator release, is inhibited.

In addition to primates, such as humans, a variety of other mammals can be treated according to the method of the present invention. For instance, mammals including, but not limited to, cows, sheep, goats, horses, dogs, cats, guinea pigs, rats or other bovine, ovine, equine, canine, feline, rodent or murine species can be treated. However, the method can also be practiced in other species, such as avian species (e.g., chickens).

Diseases and conditions associated with inflammation and infection can be treated using the compounds of the present invention. In a preferred embodiment, the disease or condition is one in which the actions of lymphocytes are to be inhibited or promoted, in order to modulate the inflammatory response.

Diseases or conditions of humans or other species which can be treated with inhibitors of chemokine receptor function, include, but are not limited to: inflammatory or allergic diseases and conditions, including respiratory allergic diseases such as asthma, particularly bronchial asthma, allergic rhinitis, hypersensitivity lung diseases, hypersensitivity pneumonitis, eosinophilic pneumonias (e.g., Loeffler's syndrome, chronic eosinophilic pneumonia), delayed-type hypersentitivity, interstitial lung diseases (ILD) (e.g., idiopathic pulmonary fibrosis, or IL)D associated with rheumatoid arthritis, systemic lupus erythematosus, ankylosing spondylitis, systemic sclerosis, Sjogren's syndrome, polymyositis or dermatomyositis); systemic anaphylaxis or hypersensitivity responses, drug allergies (e.g., to penicillin, cephalosporins), insect sting allergies; autoimmune diseases, such as rheumatoid arthritis, psoriatic arthritis, multiple sclerosis, systemic lupus erythematosus, myasthenia gravis, juvenile onset diabetes; glomerulonephritis, autoimmune thyroiditis, Behcet's disease; graft rejection (e.g., in transplantation), including allograft rejection or graft-versus-host disease; inflammatory bowel diseases, such as Crohn's disease and ulcerative colitis; spondyloarthropathies; scleroderma; psoriasis (including T-cell mediated psoriasis) and inflammatory dermatoses such an dermatitis, eczema, atopic dermatitis, allergic contact dermatitis, urticaria; vasculitis (e.g., necrotizing, cutaneous, and hypersensitivity vasculitis); eosinphilic myositis, eosinophilic fasciitis; cancers with leukocyte infiltration of the skin or organs. Other diseases or conditions in which undesirable inflammatory responses are to be inhibited can be treated, including, but not limited to, reperfusion injury, atherosclerosis, certain hematologic malignancies, cytokine-induced toxicity (e.g., septic shock, endotoxic shock), polymyositis, dermatomyositis.

Diseases or conditions of humans or other species which can be treated with modulators of chemokine receptor function, include, but are not limited to: immunosuppression, such as that in individuals with immunodeficiency syndromes such as AIDS or other viral infections, individuals undergoing radiation therapy, chemotherapy, therapy for autoimmune disease or drug therapy (e.g., corticosteroid therapy), which causes immunosuppression; immunosuppression due to congenital deficiency in receptor function or other causes; and infections diseases, such as parasitic diseases, including, but not limited to helminth infections, such as nematodes (round worms), (Trichuriasis, Enterobiasis, Ascariasis, Hookworm, Strongyloidiasis, Trichinosis, filariasis), trematodes (flukes) (Schistosomiasis, Clonorchiasis), cestodes (tape worms) (Echinococcosis, Taeniasis saginata, Cysticercosis), visceral worms, visceral larva migraines (e.g., Toxocara), eosinophilic gastroenteritis (e.g., Anisaki sp., Phocanema sp.), and cutaneous larva migraines (Ancylostona braziliense, Ancylostoma caninum). In addition, treatment of the aforementioned inflammatory, allergic and autoimmune diseases can also be contemplated for promoters of chemokine receptor function if one contemplates the delivery of sufficient compound to cause the loss of receptor expression on cells through the induction of chemokine receptor internalization or delivery of compound in a manner that results in the misdirection of the migration of cells.

The compounds of the present invention are accordingly useful in treating, preventing, ameliorating, controlling or reducing the risk of a wide variety of inflammatory and immunoregulatory disorders and diseases, allergic conditions, atopic conditions, as well as autoimmune pathologies. In a specific embodiment, the present invention is directed to the use of the subject compounds for treating, preventing, ameliorating, controlling or reducing the risk of autoimmune diseases, such as rheumatoid arthritis or psoriatic arthritis.

In another aspect, the instant invention may be used to evaluate putative specific agonists or antagonists of chemokine receptors, including CCR-2. Accordingly, the present invention is directed to the use of these compounds in the preparation and execution of screening assays for compounds that modulate the activity of chemokine receptors. For example, the compounds of this invention are useful for isolating receptor mutants, which are excellent screening tools for more potent compounds. Furthermore, the compounds of this invention are useful in establishing or determining the binding site of other compounds to chemokine receptors, e.g., by competitive inhibition. The compounds of the instant invention are also useful for the evaluation of putative specific modulators of the chemokine receptors, including CCR-2. As appreciated in the art, thorough evaluation of specific agonists and antagonists of the above chemokine receptors has been hampered by the lack of availability of non-peptidyl (metabolically resistant) compounds with high binding affinity for these receptors. Thus the compounds of this invention are commercial products to be sold for these purposes.

The present invention is further directed to a method for the manufacture of a medicament for modulating chemokine receptor activity in humans and animals comprising combining a compound of the present invention with a pharmaceutical carrier or diluent.

The present invention is further directed to the use of the present compounds in treating, preventing, ameliorating, controlling or reducing the risk of infection by a retrovirus, in particular, herpes virus or the human immunodeficiency virus (HIV) and the treatment of, and delaying of the onset of consequent pathological conditions such as AIDS. Treating AIDS or preventing or treating infection by HIV is defined as including, but not limited to, treating a wide range of states of HIV infection: AIDS, ARC (AIDS related complex), both symptomatic and asymptomatic, and actual or potential exposure to HIV. For example, the compounds of this invention are useful in treating infection by HIV after suspected past exposure to HIV by, e.g., blood transfusion, organ transplant, exchange of body fluids, bites, accidental needle stick, or exposure to patient blood during surgery.

In a further aspect of the present invention, a subject compound may be used in a method of inhibiting the binding of a chemokine to a chemokine receptor, such as CCR-2, of a target cell, which comprises contacting the target cell with an amount of the compound which is effective at inhibiting the binding of the chemokine to the chemokine receptor.

The subject treated in the methods above is a mammal, preferably a human being, male or female, in whom modulation of chemokine receptor activity is desired. “Modulation” as used herein is intended to encompass antagonism, agonism, partial antagonism, inverse agonism and/or partial agonism. In a preferred aspect of the present invention, modulation refers to antagonism of chemokine receptor activity. The term “therapeutically effective amount” means the amount of the subject compound that will elicit the biological or medical response of a tissue, system, animal or human that is being sought by the researcher, veterinarian, medical doctor or other clinician.

The term “composition” as used herein is intended to encompass a product comprising the specified ingredients in the specified amounts, as well as any product which results, directly or indirectly, from combination of the specified ingredients in the specified amounts. By “pharmaceutically acceptable” it is meant the carrier, diluent or excipient must be compatible with the other ingredients of the formulation and not deleterious to the recipient thereof.

The terms “administration of” and or “administering a” compound should be understood to mean providing a compound of the invention to the individual in need of treatment.

As used herein, the term “treatment” refers both to the treatment and to the prevention or prophylactic therapy of the aforementioned conditions.

Combined therapy to modulate chemokine receptor activity for thereby treating, preventing, ameliorating, controlling or reducing the risk of inflammatory and immunoregulatory disorders and diseases, including asthma and allergic diseases, as well as autoimmune pathologies such as rheumatoid arthritis and atherosclerosis, and those pathologies noted above is illustrated by the combination of the compounds of this invention and other compounds which are known for such utilities.

For example, in treating, preventing, ameliorating, controlling or reducing the risk of inflammation, the present compounds may be used in conjunction with an antiinflammatory or analgesic agent such as an opiate agonist, a lipoxygenase inhibitor, such as an inhibitor of 5-lipoxygenase, a cyclooxygenase inhibitor, such as a cyclooxygenase-2 inhibitor, an interleukin inhibitor, such as an interleukin-1 inhibitor, an NMDA antagonist, an inhibitor of nitric oxide or an inhibitor of the synthesis of nitric oxide, a non-steroidal antiinflammatory agent, or a cytokine-suppressing antiinflammatory agent, for example with a compound such as acetaminophen, aspirin, codeine, embrel, fentanyl, ibuprofen, indomethacin, ketorolac, morphine, naproxen, phenacetin, piroxicam, a steroidal analgesic, sufentanyl, sunlindac, tenidap, and the like. Similarly, the instant compounds may be administered with a pain reliever; a potentiator such as caffeine, an H2-antagonist, simethicone, aluminum or magnesium hydroxide; a decongestant such as phenylephrine, phenylpropanolamine, pseudophedrine, oxymetazoline, ephinephrine, naphazoline, xylometazoline, propylhexedrine, or levo-desoxy-ephedrine; an antiitussive such as codeine, hydrocodone, caramiphen, carbetapentane, or dextramethorphan; a diuretic; and a sedating or non-sedating antihistamine.

Likewise, compounds of the present invention may be used in combination with other drugs that are used in the treatment/prevention/suppression or amelioration of the diseases or conditions for which compounds of the present invention are useful. Such other drugs may be administered, by a route and in an amount commonly used therefor, contemporaneously or sequentially with a compound of the present invention. When a compound of the present invention is used contemporaneously with one or more other drugs, a pharmaceutical composition containing such other drugs in addition to the compound of the present invention is preferred. Accordingly, the pharmaceutical compositions of the present invention include those that also contain one or more other active ingredients, in addition to a compound of the present invention.

Examples of other active ingredients that may be combined with a compound of the present invention, either administered separately or in the same pharmaceutical compositions, include, but are not limited to: (a) VLA-4 antagonists such as those described in U.S. Pat. No. 5,510,332, WO095/15973, WO096/01644, WO096/06108, WO096/20216, WO096/22966, WO096/31206, WO096/40781, WO097/03094, WO097/02289, WO 98/42656, WO098/53814, WO098/53817, WO098/53818, WO098/54207, and WO098/58902; (b) steroids such as beclomethasone, methylprednisolone, betamethasone, prednisone, dexamethasone, and hydrocortisone; (c) immunosuppressants such as cyclosporin, tacrolimus, rapamycin and other FK-506 type immunosuppressants; (d) antihistamines (HI-histamine antagonists) such as bromopheniramine, chlorpheniramine, dexchlorpheniramine, triprolidine, clemastine, diphenhydramine, diphenylpyraline, tripelennamine, hydroxyzine, methdilazine, promethazine, trimeprazine, azatadine, cyproheptadine, antazoline, pheniramine pyrilamine, astemizole, terfenadine, loratadine, desloratadine, cetirizine, fexofenadine, descarboethoxyloratadine, and the like; (e) non-steroidal anti-asthmatics such as β2-agonists (terbutaline, metaproterenol, fenoterol, isoetharine, albuterol, bitolterol, and pirbuterol), theophylline, cromolyn sodium, atropine, ipratropium bromide, leukotriene antagonists (zafirlukast, montelukast, pranlukast, iralukast, pobilukast, SKB-106,203), leukotriene biosynthesis inhibitors (zileuton, BAY-1005); (f) non-steroidal antiinflammatory agents (NSAIDs) such as propionic acid derivatives (alminoprofen, benoxaprofen, bucloxic acid, carprofen, fenbufen, fenoprofen, fluprofen, flurbiprofen, ibuprofen, indoprofen, ketoprofen, miroprofen, naproxen, oxaprozin, pirprofen, pranoprofen, suprofen, tiaprofenic acid, and tioxaprofen), acetic acid derivatives (indomethacin, acemetacin, alclofenac, clidanac, diclofenac, fenclofenac, fenclozic acid, fentiazac, furofenac, ibufenac, isoxepac, oxpinac, sulindac, tiopinac, tolmetin, zidometacin, and zomepirac), fenamic acid derivatives (flufenamic acid, meclofenamic acid, mefenamic acid, niflumic acid and tolfenamic acid), biphenylcarboxylic acid derivatives (diflunisal and flufenisal), oxicams (isoxicam, piroxicam, sudoxicam and tenoxican), salicylates (acetyl salicylic acid, sulfasalazine) and the pyrazolones (apazone, bezpiperylon, feprazone, mofebutazone, oxyphenbutazone, phenylbutazone); (g) cyclooxygenase-2 (COX-2) inhibitors; (h) inhibitors of phosphodiesterase type IV (PDE-IV); (i) other antagonists of the chemokine receptors, especially CCR-1, CCR-2, CCR-3, CXCR-3 and CCR-5; (j) cholesterol lowering agents such as HMG-CoA reductase inhibitors (lovastatin, simvastatin and pravastatin, fluvastatin, atorvastatin, rosuvastatin, and other statins), sequestrants (cholestyramine and colestipol), cholesterol absorption inhibitors (ezetimibe), nicotinic acid, fenofibric acid derivatives (gemfibrozil, clofibrat, fenofibrate and benzafibrate), and probucol; (k) anti-diabetic agents such as insulin, sulfonylureas, biguanides (metformin), α-glucosidase inhibitors (acarbose) and glitazones (troglitazone and pioglitazone); (l) preparations of interferon beta (interferon beta-1α, interferon beta-1β); (m) other compounds such as 5-aminosalicylic acid and prodrugs thereof, antimetabolites such as azathioprine and 6-mercaptopurine, and cytotoxic cancer chemotherapeutic agents.

The weight ratio of the compound of the present invention to the second active ingredient may be varied and will depend upon the effective dose of each ingredient. Generally, an effective dose of each will be used. Thus, for example, when a compound of the present invention is combined with an NSAID the weight ratio of the compound of the present invention to the NSAID will generally range from about 1000:1 to about 1:1000, preferably about 200:1 to about 1:200. Combinations of a compound of the present invention and other active ingredients will generally also be within the aforementioned range, but in each case, an effective dose of each active ingredient should be used.

In such combinations the compound of the present invention and other active agents may be administered separately or in conjunction. In addition, the administration of one element may be prior to, concurrent to, or subsequent to the administration of other agent(s).

The compounds of the present invention may be administered by oral, parenteral (e.g., intramuscular, intraperitoneal, intravenous, ICV, intracisternal injection or infusion, subcutaneous injection, or implant), by inhalation spray, nasal, vaginal, rectal, sublingual, or topical routes of administration and may be formulated, alone or together, in suitable dosage unit formulations containing conventional non-toxic pharmaceutically acceptable carriers, adjuvants and vehicles appropriate for each route of administration. In addition to the treatment of warm-blooded animals such as mice, rats, horses, cattle, sheep, dogs, cats, monkeys, etc., the compounds of the invention are effective for use in humans.

The pharmaceutical compositions for the administration of the compounds of this invention may conveniently be presented in dosage unit form and may be prepared by any of the methods well known in the art of pharmacy. All methods include the step of bringing the active ingredient into association with the carrier which constitutes one or more accessory ingredients. In general, the pharmaceutical compositions are prepared by uniformly and intimately bringing the active ingredient into association with a liquid carrier or a finely divided solid carrier or both, and then, if necessary, shaping the product into the desired formulation. In the pharmaceutical composition the active object compound is included in an amount sufficient to produce the desired effect upon the process or condition of diseases. As used herein, the term “composition” is intended to encompass a product comprising the specified ingredients in the specified amounts, as well as any product which results, directly or indirectly, from combination of the specified ingredients in the specified amounts.

The pharmaceutical compositions containing the active ingredient may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the techniques described in the U.S. Pat. Nos. 4,256,108; 4,166,452; and 4,265,874 to form osmotic therapeutic tablets for control release.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin, or olive oil.

Aqueous suspensions contain the active materials in admixture with excipients suitable for the manufacture of aqueous suspensions. Such excipients are suspending agents, for example sodium carboxymethylcellulose, methylcellulose, hydroxy- propylmethylcellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethylene-oxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, such as sucrose or saccharin.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

The pharmaceutical compositions of the invention may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative and flavoring and coloring agents.

The pharmaceutical compositions may be in the form of a sterile injectable aqueous or oleagenous suspension. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. Among the acceptable vehicles and solvents that may be employed-are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The compounds of the present invention may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials are cocoa butter and polyethylene glycols.

For topical use, creams, ointments, jellies, solutions or suspensions, etc., containing the compounds of the present invention are employed. (For purposes of this application, topical application shall include mouthwashes and gargles.)

The pharmaceutical composition and method of the present invention may further comprise other therapeutically active compounds as noted herein which are usually applied in the treatment of the above mentioned pathological conditions.

In treating, preventing, ameliorating, controlling or reducing the risk of conditions which require chemokine receptor modulation an appropriate dosage level will generally be about 0.01 to 500 mg per kg patient body weight per day which can be administered in single or multiple doses. Preferably, the dosage level will be about 0.1 to about 250 mg/kg per day; more preferably about 0.5 to about 100 mg/kg per day. A suitable dosage level may be about 0.01 to 250 mg/kg per day, about 0.05 to 100 mg/kg per day, or about 0.1 to 50 mg/kg per day. Within this range the dosage may be 0.05 to 0.5, 0.5 to 5 or 5 to 50 mg/kg per day. For oral administration, the compositions are preferably provided in the form of tablets containing 1.0 to 1000 milligrams of the active ingredient, preferably 2.0 to 500, more preferably 3.0 to 200, particularly 1, 5, 10, 15, 20, 25, 30, 50, 75, 100, 125, 150, 175, 200, 250, 300, 400, 500, 600, 750, 800, 900, and 1000 milligrams of the active ingredient for the symptomatic adjustment of the dosage to the patient to be treated. The compounds may be administered on a regimen of 1 to 4 times per day, preferably once or twice per day.

It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

### Schemes

Several methods for preparing the compounds of this invention are illustrated in the following Schemes and Examples. Starting materials are commercially available, made by known procedures, or prepared as illustrated herein.

One of the principal routes used for preparation of compounds within the scope of the instant invention which bear a 1,1,3-trisubstituted cyclopentane framework 1-5 is depicted in Scheme 1. According to this route, keto acids 1-1 (preparation described in Schemes 2A, 2B, 2C, and 2D) is coupled to amines 1-2 (either commercially available or synthesized according to literature procedures). This can be accomplished in various ways, including by first converting the acid to its acid chloride with a reagent such as oxalyl chloride, and then combining with amine 1-2 in the presence of a base such as triethylamine. Reductive amination of 1-3 with an amine (NH₂—R⁸) (available commercially or synthesized according to literature procedures) using, for example, NaB(OAc)₃H or NaBH₃CN as the reducing agent to give final products of the form 1-5a. These compounds can be further modified to make different final compounds of the form 1-5b, by reductive alkylation with aldehydes or ketones (O═R⁷). Compounds 1-5 are often obtained as a mixture of cis and trans isomers. When 1-1 is a single stereoisomer only 2 possible isomers of 1-5 can result (cis and trans); these can be separated by a variety of methods, including by preparative TLC, flash chromatography, MPLC, or by HPLC using a column with a chiral stationary phase. When 1-1 is racemic, a total of at least 4 possible isomers of 1-5 can be obtained. Again, these may be separated by HPLC using a column with a chiral stationary phase, or by a combination of the methods above.

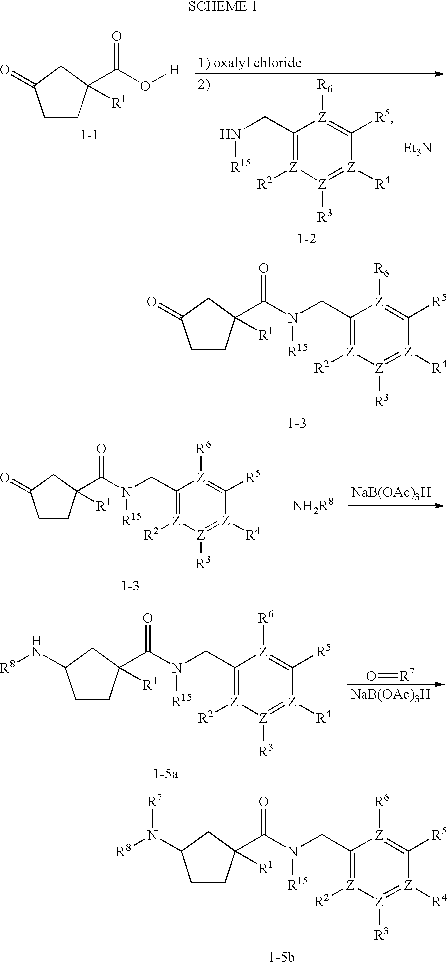

Furthermore, compounds 1-5 can themselves be modified to give new chemokine receptor modulators 1-5.1. For example, an ester functional group within a compound 1-5 can be hydrolyzed to the corresponding carboxylic acid, which also can be a chemokine receptor modulator.

As an alternate route to chemokine modulators 1-5 is shown in Scheme 1A. As depicted in this scheme, the keto-ester 1-6 (where R³⁰ is an approporiate alkyl group) could be reductively aminated with an amine to form the amino ester 1-7 under a variety of conditions, including sodium triacetoxyborohydride or sodium cyanoborohydride. Alkylation of the ester 1-7 with an alkylating agent such as an alkyl chloride, bromide or iodide in the presence of an appropriate base such as lithium bis(trimethylsilyl)amide, affords the intermediate esters 1-8. These esters formed in the above mentioned transformations represent in general a mixture of 1,3-cis- and 1,3-trans-diastereoisomers, which could be separated into respective diastereoisomeric pairs using column chromatography. A similar diastereoisomeric separation could be also accomplished later, after the esters 1-8 were hydrolytically cleaved to yield the respective acids 1-9. This hydrolysis was readily accomplished under usual conditions, including lithium, sodium or potassium hydroxide, at ambient to elevated temperatures, depending on the nature of the ester group and substituent R¹. These diastereoisomers could be separated by crystallization from a variety of solvents, taking advantage of the finding, that the cis-diastereoisomeric acids are less soluble, when compared to their trans-epimers.

The compounds of formula 1-5c are then formed from the acids 1-9 and benzylamine derivatives 1-2 under standard amide-bond forming reaction conditions, including carbodiimide reagents, such as DCC, EDC and catalysts such as DMAP, HOAT or HOBT.

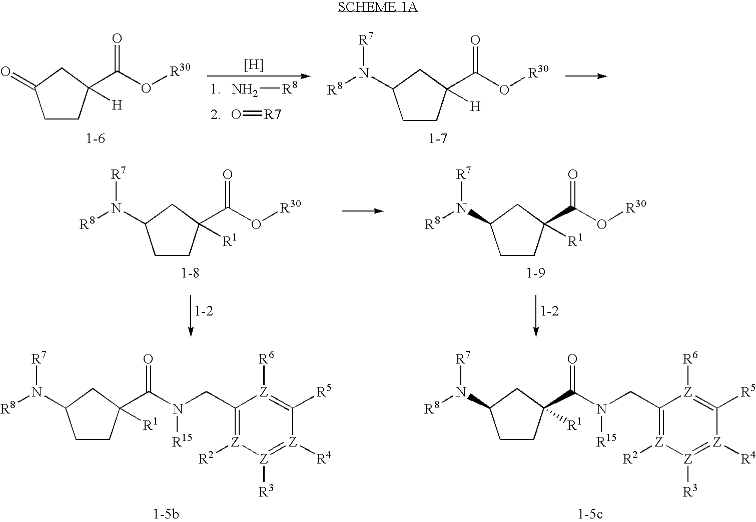

Additionally, Intermediate 1-3 can also be resolved by Chiral HPLC to give 1-3.1 and 1-3.2 (Scheme 1B). This then would give cis isomers 1-5a.1 or 1-5b.1 and the trans isomers 1-5a.2 or 1-5b.2.

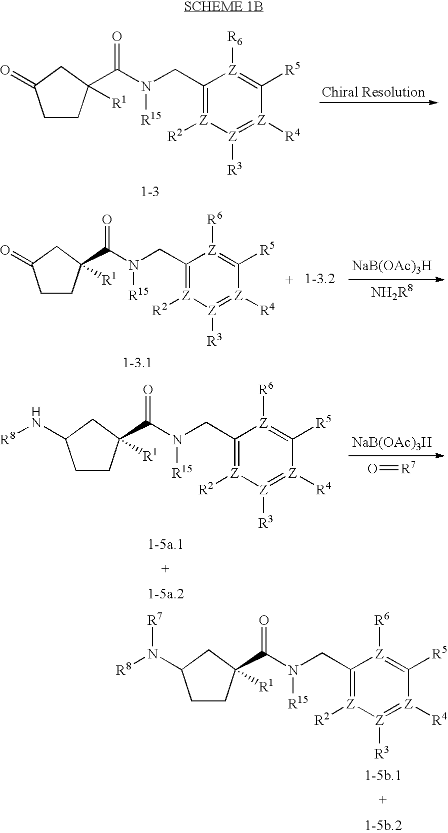

Another principal route for the synthesis of chemokine receptor modulators of the form 1-5a and 1-5b is depicted in Scheme IC. According to this route, intermediate 1-10 (described in Scheme 2C) is condensed with amine 1-2 using a peptide coupling reagent such as EDC to give 1-11. The Boc protecting group is removed under standard conditions such as with HCl in a solvent such as dioxane followed by treatment of the resulting amine 1-12 with an aldehyde or ketone (O═R⁸) in the presence of a reducing agent such as sodium triacetoxyborohydride leads to 1-5a. Further reductive amination with a ketone or aldehyde (R═R⁷) gives rise to new chemokine modulators 1-5b.

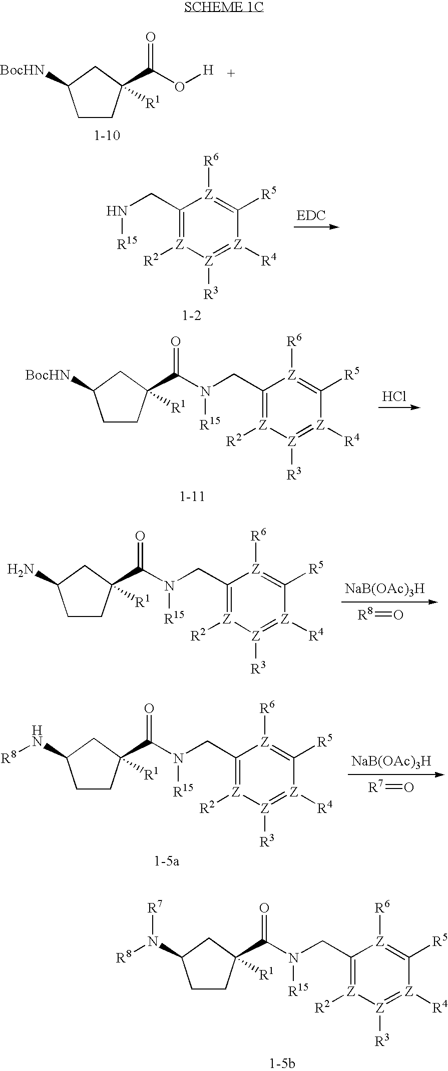

Another principal route for the synthesis of chemokine receptor modulators of the form 1-5c is depicted in Scheme 1D. According to this route, intermediate 1-12 (described in Scheme 1C) is alkylated with an alkyl bromide using an appropriate base to give new chemokine modulators 1-5a.

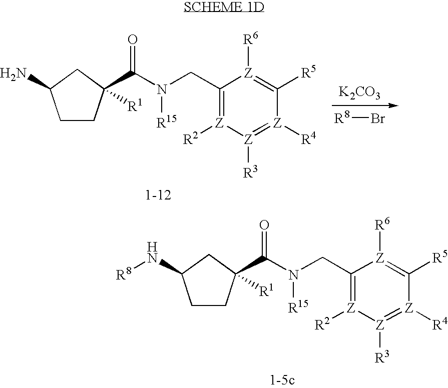

One of the principal routes used for the preparation of Intermediate 1-1 and Intermediate 1-6 is outlined in Scheme 2A. According to this route, 3-oxocyclopentanecarboxylic acid (2-1), which can be synthesized following a known procedure (Stetter, H., Kuhlman, H., Liebigs Ann. Chim., 1979, 944) is esterified under standard conditions. When R³⁰ represents a tert-Butyl group, the respective ester 1-6 can be prepared by reacting the appropriate alcohol, in this case tert-butanol, with acid 2-1 in the presence of sulfuric acid. Protection of the oxo-group in 2-1 can be achieved by a number of ways (Greene, T., Wuts, P. G. M., Protective Groups in Organic Chemistry, John Wiley & Sons, Inc., New York, N.Y. 1991). The particularly suitable dimethyl acetal protecting group can be introduced using trimethylorthoformate as a reagent in a suitable solvent such as dichloromethane and methyl alcohol in the presence of an acidic catalyst. Alternatively, in the case of R³⁰ being a methyl group, the acid 2-1 can be converted to 2-3 directly by using trimethylorthoformate and an acidic catalyst, such as para-toluenesulfonic acid. An alkylation of esters 2-3 with an alkylating agent such as an alkyl chloride, bromide or iodide in the presence of an appropriate base such as lithium diisopropylamide, produces intermediates 2-4. The ester protecting group present in 2-4 can be removed in a number of ways, depending on the nature of the ester. Methyl esters (R³⁰=methyl) can be hydrolyzed in the presence of an acid or base at ambient or elevated temperatures, whereas tert-butyl esters (R³⁰=tert-butyl) can be easily cleaved under acidic conditions. Under these conditions, the dimethyl acetal is simultaneously deprotected to give 1-1.

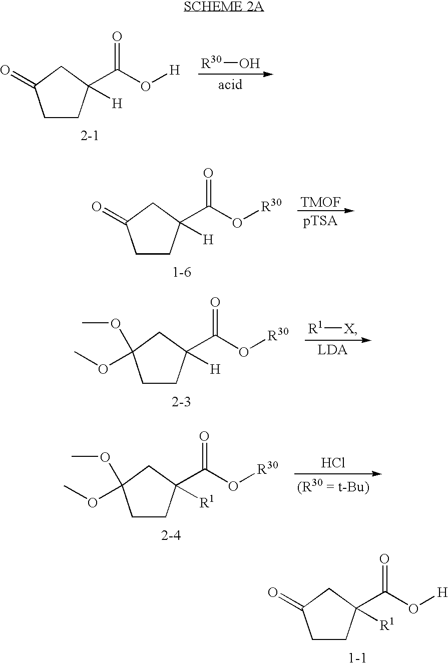

Intermediate 1-1 can be prepared as a single stereoisomer (1-1a) in various ways including those depicted in Schemes 2B and 2C. According to Scheme 2B, racemic 1-1 can be converted to its benzyl ester. There are many ways to effect this esterification, one of which being by a sequence involving conversion to the corresponding acid chloride with, for example oxalyl chloride, followed by treatment with benzyl alcohol in the presence of a base such as triethylamine. Then the racemic benzyl ester 2-5 can be separated by chiral preparative HPLC to give 2-5a as a single stereoisomer. Removal of the benzyl group to give the chiral ketoacid 1-1a can be accomplished in several ways. One convenient way is by hydrogenolysis in the presence of a catalyst such as Pd/C.

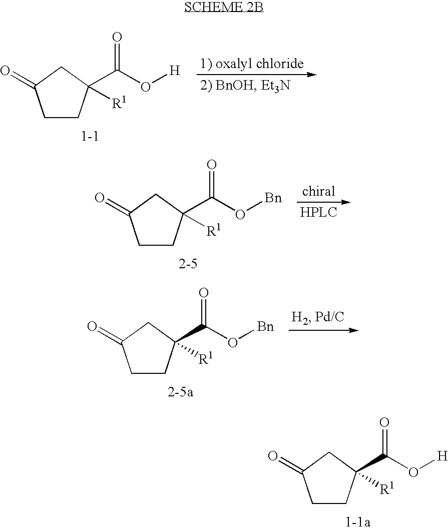

According to Scheme 2C, chiral ketoacid intermediate 1-1a can be prepared starting from commercially available optically pure amino acid 2-6. Protection of the carboxylic acid group can be achieved in a variety of ways. When R³⁰ is methyl, esterification can be accomplished by treatment with methanol in the presence of an acid catalyst such as HCl. Treatment with Boc₂O results in protection of the amine group of 2-7. Stereoselective alkylation of ester 2-8 with an alkylating agent such as an alkyl chloride, bromide or iodide in the presence of an appropriate base such as lithium bis(trimethylsilyl)amide, produces intermediates 2-9. Hydrogenation in the presence of a catalyst such as Pd/C affords 2-10. Hydrolysis of the ester to give 1-10 can be achieved under standard conditions depending on the R³⁰ group. For example, when R³⁰ is methyl (methyl ester), hydrolysis can be accomplished by treatment with a base such as sodium hydroxide, lithium hydroxide, or potassium hydroxide, with or without heating. The Boc protecting group can be removed under standard acidic conditions, such as with HCl in a solvent such as dioxane, or with TFA. Oxidation of 2-11 to give 1-1a (as a single stereoisomer if constituent R¹ is achiral, or as a mixture of stereoisomers if constituent R¹ has a chiral center) can be achieved in several ways, including by treatment with NBS, followed by treatment with sodium methoxide.

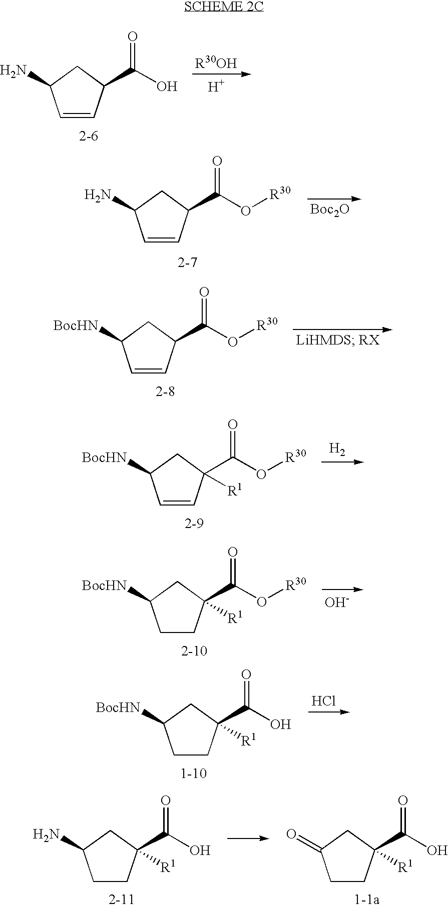

Another principal route to synthesize compounds within the scope of the instant invention is detailed in Scheme 3A and 3B.

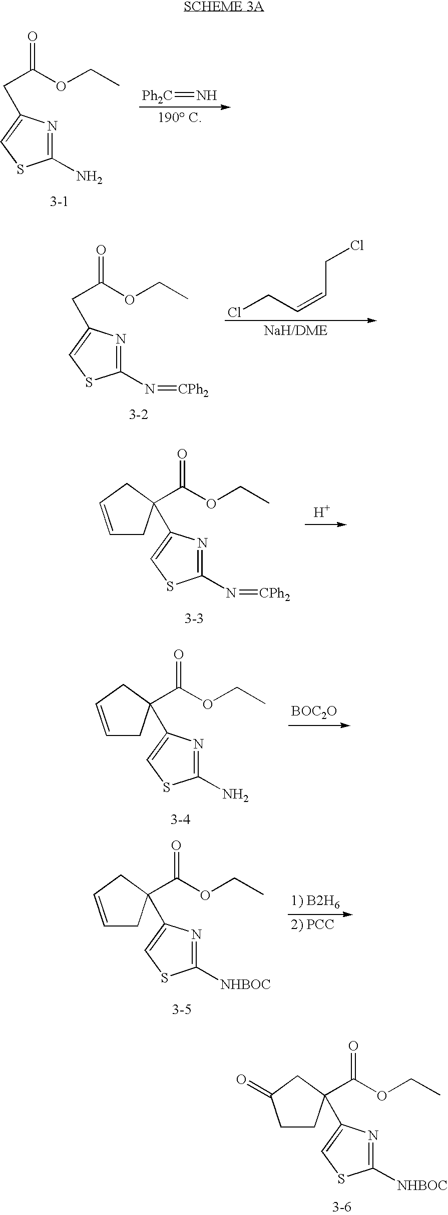

According to this, the commercially available ethyl aminothiazole acetate 3-1 is treated with benzophenone imine, preferably at elevated temperature. The enolate, generated from ester 3-2 with a strong base, e.g. sodium hydride is then double alkylated with 1,4-dichloro-2-butene in a suitable solvent, such as dimethoxyethane preferably in the presence of an additional co-solvent (e.g. DMPU) to suppress undesired side-reactions. The cleavage of the Schiff base 3-3 is accomplished as described previously and the amino group in 3-4 is protected by treatment with BOC₂O in the presence of a catalytic amount of DMAP 3-5. Addition of borane to the double bond (see March, J. Advanced Organic Chemistry, 4^{th} edition, John Wiley & Sons Inc., New York, p. 702-707) is followed by a direct pyridinium chlorochromate mediated oxidation of the formed adduct to produce ketones 3-6 directly, in fair yield.

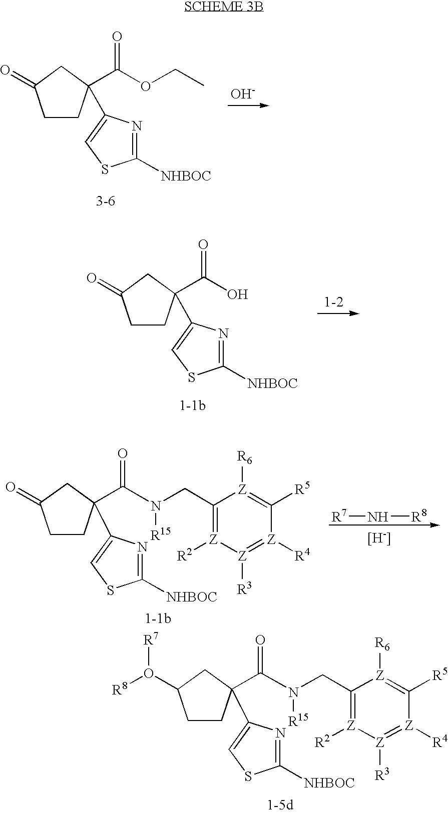

The ester group present in intermediates 3-6 is then removed by a base catalyzed hydrolysis, and the acids I-1b are coupled to amines 1-2 as discussed previously. The last step in preparation of final compounds 1-5d is a reductive amination of the ketone with amines as detailed above. Similarly to the case described in Schemes 1 this synthetic sequence produces mixtures of diastereoisomers, and their separation can be accomplished using chromatography on normal-, reverse-, or chiral phases.

There are several more specialized ways to synthesize compounds of the formula I. These routes are elaborated in the experimental section. In some cases the order of carrying out the foregoing reaction schemes may be varied to facilitate the reaction or to avoid unwanted reaction products. The following examples are provided for the purpose of further illustration only and are not intended to be limitations on the disclosed invention.

Concentration of solutions was generally carried out on a rotary evaporator under reduced pressure. Flash chromatography was carried out on silica gel (230-400 mesh). NMR spectra were obtained in CDCl₃ solution unless otherwise noted. Coupling constants (J) are in hertz (Hz). Abbreviations: ethyl acetate (EA), diethyl ether (ether), triethylamine (TEA), N,N-diisopropylethylamine (DIEA) saturated aqueous (sat'd), room temperature (rt), hour(s) (h), minute(s) (min).

The following are representative Procedures for the preparation of the compounds used in the following Examples or which can be substituted for the compounds used in the following Examples which may not be commercially available

### INTERMEDIATES & EXAMPLES

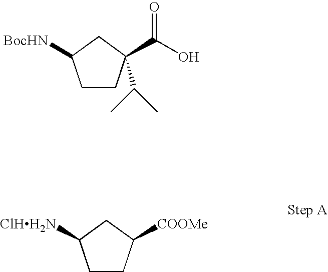

A mixture of (1S)-(+)-2-azabicyclo[2.2.1]hept-5-en-3-one (10.3 g, 94.4 mmol) in EtOAc (200 mL) and 10% Pd/C (0.5 gm), was hydrogenated at room temperature under a hydrogen balloon. After 24 h the reaction mixture was filtered and evaporated leaving behind 10.4 g (100%) of a product that was taken in 250 mL methanol and HCl (12M, 6 ML). The resultant mixture was stirred at RT, until the reaction was complete (72 h). Evaporation of methanol followed by drying under high vacuum, yielded the title compound as an off white solid (16.0 g, 96%). ¹H NMR (D₂O, 500 MHz): 3.70 (s, 3H), 3.01 (m, 1H), 2.38 (m, 1H), 2.16-1.73 (m, 6H).

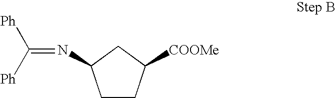

To a suspension of the intermediate from step A (10.2 g, 56.8 mmol) in dry dichloromethane (200 mL) was added benzophenone imine (10.2 g, 56.8 mmol) at room temperature and the resultant mixture was stirred for 24 h. The reaction mixture was filtered and the filtrate was evaporated, to leave behind a yellow oil that was triturated with ether (100 mL), filtered and evaporated. This operation was repeated twice to ensure that the product was free of ammonium chloride impurities. The resultant oil was thoroughly dried under vacuum to yield the title compound (18.03 g, >100%) and required no further purification. ¹H NMR (CDCl₃, 500 MHz): 7.5-7.18 (m, 10H), 3.75 (m, 1H), 3.7 (s, 3H), 2.78 (m, 1H), 2.26-1.71 (m, 6H).

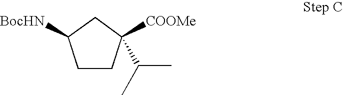

To a solution of LDA (prepared from diisopropylamine (7.7 g, 76.1 mmol) and n-butyllithium (30.4 mL, 2.5 M in hexane, 76 mmol) in THF (120 mL) at −78° C. was added the ester from Step B (18.0 g, 58.6 mmol). The resultant burgundy colored solution was stirred for 20 min. after which it was quenched with 2-iodopropane (14.9 gm, 88.0 mmol). The reaction mixture was gradually warmed over 3 h to 0° C. and this temperature was maintained for an additional 3 h. Reaction was quenched with water and extracted with EtOAc. The organic layer was washed with water, brine, dried (anhydrous magnesium sulfate) and concentrated to yield an oil. To the solution of the crude Schiff base (20.0 g) in THF (100 mL) was added HCl (5.0 mL, 12 M) and was allowed to stir at room temperature for 3 h. After the removal of all volatiles, the hydrochloride salt was taken up into dichloromethane (250 mL), and a saturated solution of sodium bicarbonate (250 mL) and di-tert-butyl dicarbonate (26.0 g, 1.4 Eq.) were added. The resultant mixture was vigorously stirred overnight at RT. The organic layer was separated and washed with water, brine, dried (anhydrous magnesium sulfate) and concentrated to yield an oil. Purification by flash column chromatography (eluent:hexane:EtOAc/19:1) gave the desired product (4.91 g, 30%). ¹H NMR (500 MHz, CDCl₃): 4.79 (br, 1H), 4.01 (m, 1H), 3.71 (s, 3H), 2.18-1.60 (m, 6H), 1.44 (s, 9H), 0.87 (d, J=6.9 Hz, 3), 0.86 (d, J=6.9 Hz, 3H).

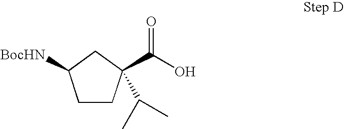

To a solution of the ester from the previous step (4.91 g, 17.2 mmol) in MeOH (100 mL) was added a solution of LiOH (3.6 g, 85 mmol) in water (20 mL) and THF (10 mL). The resultant mixture was heated at 80° C. until the reaction was complete (18 h). Methanol was removed in vacuo and the crude product was taken up with water/EtOAc (200 mL, 1:4) and cooled to 0° C. The acidity of the mixture was adjusted to pH 6. The EtOAc layer was separated, washed with water, brine, dried (anhydrous magnesium sulfate) and concentrated to yield an oil. Purification by flash column chromatography (eluent:hexane:EtOAc/1:1+2% AcOH) gave Intermediate 1 (3.9 g, 84%). ¹H NMR (500 MHz, CDCl₃): 11.36 (br, 1H), 6.49 (br, 1H), 4.83 (m, 1H), 3.71 (s, 3H), 2.30-1.55 (m, 6H), 1.46 (s, 9H), 0.94 (d, J=6.9 Hz, 3H), 0.933 (d, J=6.9 Hz, 3H).

### Intermediate 2

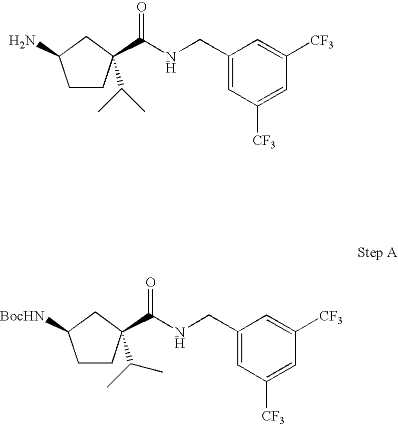

To a stirred solution of Intermediate 1 (2.09 g, 7.71 mmol), 1-ethyl-3-(3-dimethylaminopropyl)carbodiimide (2.96 g, 15.4 mmol) in DCM (100 mL), was added 3,5-bis(trifluoro)benzylamine hydrochloride (2.26 g, 8.10 mmol), diisopropylethylamine (1.05 g, 8.10 mmol), and 1-hydroxy-7-azabenzotriazole (1.15 g, 8.48 mmol). The reaction was stirred at room temperature for 18 h before being diluted with DCM and washed twice with aqueous 1 N HCl, once with saturated aqueous sodium bicarbonate, and once with brine. The organic layer was dried over MgSO₄, filtered and concentrated under reduced pressure. The product was purified by medium pressure liquid chromatography (silica gel, 60% EA/Hexanes) to give 2.23 g of a colorless oil which was used directly in Step B.

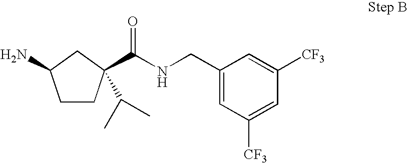

The product from Step A was dissolved in hydrogen chloride (4 N solution in dioxane, 25 mL) and stirred at room temperature. After 1.5 h the reaction was concentrated under reduced pressure to give 1.79 g of a white solid (54% over 2 steps). ESI-MS calc. for C18H22F6N2O: 396.4; found 397.2 (M+H).

### Example 1

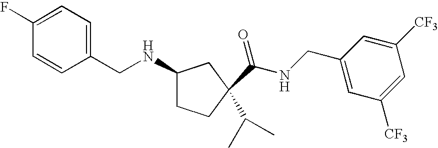

To a stirred suspension of Intermediate 2 (hydrochloride, 33 mg, 0.076 mmol) in 2 mL of methylene chloride at room temperature was added 16 μL (0.95 mmol) of diisopropylethylamine and 9.5 mg (0.076 mmol) of 4-fluorobenzaldehyde. To the reaction mixture was added 4 beads of molecular sieves 4 Å followed by 24 mg of Na(OAc)₃BH. After stirring overnight the reaction mixture was evaporated and the product (21 mg) was isolated by preparative TLC on silica gel plates (CH₂Cl₂:CH₃OH:NH₄OH/85:15:1). ¹H NMR (400 Mz, CD₃OD): 0.85 (t, 6H), 3.68 (q, 2H), 4.48 (q, 2H), 7.00 (m, 2H), 7.29 (q, 2H), 7.82 (s, 1H), 7.87 (s, 2H). LC MS for C₂₅H₂₇F₇N₂O [M+H]⁺ calc. 505, found 505.

### Examples 2-14

Following the procedure described in Example 1, a series of analogous target compounds were synthesized. Their structure and MS-characteristics are summarized in the following Table.

TABLE 1

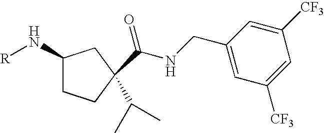

| | | Molecular | Calc'd | Found |
|---|---|---|---|---|
| Ex. | R | Formula | [M + H]⁺ | [M + H]⁺ |
| 2 | 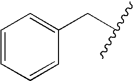 | C₂₅H₂₈F₆N₂O | 487.21 | 487 |
| 3 | 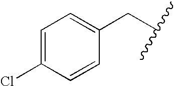 | C₂₅H₂₈ClF₆N₂O | 521.17 | 521 |
| 4 | 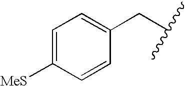 | C₂₆H₃₀F₆N₂OS | 533.20 | 533 |
| 5 | 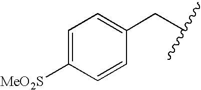 | C₂₆H₃₀F₆N₂O₃S | 565.19 | 565 |
| 6 | 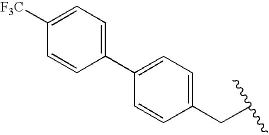 | C₃₂H₃₁F₉N₂O | 631.23 | 631 |
| 7 | 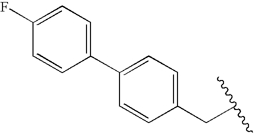 | C₃₁H₃₁F₇N₂O | 481.23 | 581 |
| 8 | 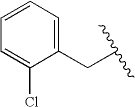 | C₂₅H₂₇ClF₆N₂O | 521.17 | 521 |
| 9 | 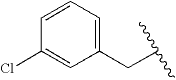 | C₂₅H₂₇ClF₆N₂O | 521.17 | 521 |
| 10 | 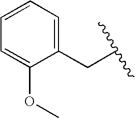 | C₂₆H₃₀F₆N₂O₂ | 517.22 | 517 |
| 11 | 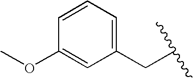 | C₂₆H₃₀F₆N₂O | 501.23 | 517 |
| 12 | 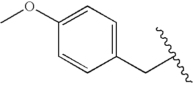 | C₂₆H₃₀F₆N₂O | 501.23 | 517 |
| 13 | 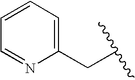 | C₂₄H₂₇F₆N₃O | 488.21 | 488 |
| 14 | 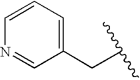 | C₂₄H₂₇F₆N₃O | 488.21 | 488 |

### Intermediate 3

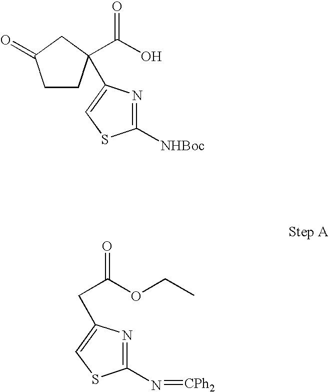

A neat mixture of 54 g (0.29 mole) ethyl (2-aminothiazol-4-yl)acetate and 50 g (0.28 mole) benzophenone imine was stirred at 190° C. for 5 h and then cooled to room temperature and diluted with 100 mL of CH₂Cl₂. The entire mixture was transferred onto a silica gel column and eluted with 20% EtOAc/Hexane. The title compound was obtained as light-yellow solid (70 g, 69% yield). ¹H NMR (300 MHz, CDCl₃): 1.26 (t, 3H), 3.74 (s, 2H), 4.15 (q, 2H), 6.87 (s, 1H), 77.25-7.86 (m, 10 H). LC MS for C20H18N2O2S for [M+H]⁺ calc.351, found 351.

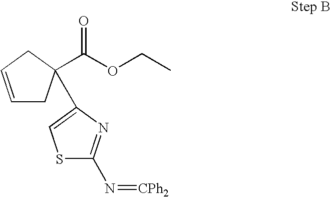

To a mixture of 35 g (0.10 mol) of ethyl (2-diphenylmethyleneamino-thiazol-4-yl)acetate (Step A, Example 195), cis-1,3-dichloro-2-butene (13 mL, 0.11 mol) in 500 mL of DME at room temperature was added in multiple portions solid NaH (60% oil, 10 g, 0.25 mol). The resulting mixture was stirred for 2 days, poured into 2000 mL of ice-water and extracted with 1500 mL of ether. The ether layer was washed with water (3×500 mL), dried over Na₂SO₄ and evaporated. Flash chromatography (silica gel, 5% EtOAc/Hexane) afforded the title compound as an oil (24 g, 59%). ¹H NMR (300 MHz, CDCl₃): 1.20 (t, 3H), 2.87 (d, 2H), 3.19 (d, 2H), 4.14 (q, 2H), 5.29 (s, 2H), 6.71 (s, 1H), 7.26-7.81 (m, 10H). LC MS for C24H22N2O2S for [M+H]⁺ calc.403, found 403.

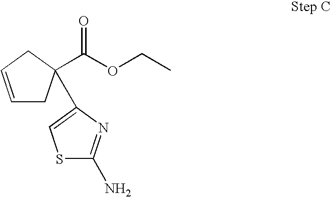

24 g (0.059 mol) of ethyl 1-(2-diphenylmethyleneamino-thiazol-4-yl)-3-cyclopentenecarboxylate (Step B, Example 195) was dissolved in 100 mL of 4 N HCl/dioxane. After 1 h, 1.8 mL of water was added. The mixture was stirred for 3 h and evaporated to dryness. The residue was dissolved in 100 mL of CH₂Cl₂ and 15 mL of DIEA was added. The entire mixture was dumped onto a silica gel column, eluted with 20% EtOAc/hexanes to remove benzophenone, then eluted with 40% EtOAc/hexane to give the title compound as a light yellow solid (12.0 g, 85%). ¹H NMR (300 MHz, CDCl₃): 1.19 (t, 3H), 2.79 (d, 12H), 3.15 (d, 2H), 4.13 (q, 2H), 5.66 (s, 2H), 5.82 (wide, 2H), 6.19 (s, 1H).

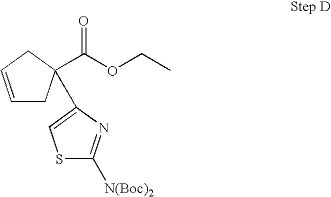

A mixture of 12 g (50 mmol) of ethyl 1-(2-amino-thiazol-4-yl)-3-cyclopentenecarboxylate (Step C, Example 195), 28 g (0.13 mol) of di-tert-butyl dicarbonate and 0.6 g of DMAP in 250 mL of CH₂Cl₂ was stirred overnight, and evaporated. The title compound (21.0 g, 96%) was obtained as a yellow oil after flash chromatography purification on silica gel (10% EtOAc/Hexane). ¹H NMR (300 MHz, CDCl₃): 1.18 (t, 3H), 1.49 (d, 18H), 2.88 (d, 2H), 3.18 (d, 2H), 4.13 (q, 2H), 5.65 (s, 2H), 6.83 (s, 1H). LC MS for C21H30N2O6S for [M+H]⁺ calc.439, found 439.

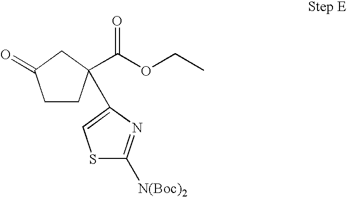

To a solution of 13 g (30 mmol) of ethyl 1-(2-Bis-Boc-amino-thiazol-4-yl)-3-cyclopentenecarboxylate (Step D, Example 195) in 50 mL of anhydrous ether at −78° C. was added dropwise a solution of borane-dimethyl sulfide in THF (14 mL, 0.024 mmol). The cooling bath was removed and the mixture was stirred at room temperature for 3 h, diluted with 250 mL of CH₂Cl₂, and 25 g of sodium acetate and 55 g of PCC were added. The mixture was stirred overnight. The entire mixture was dumped onto a silica gel column and eluted with in 10% EtOAc/hexane and then 30% EtOAc/hexane. Two components were obtained. The fast-eluted isomer (yellow oil, 6.0 g) was identified as the title compound. ¹H NMR (300 Mz, CDCl₃): 1.21 (t, 3H), 1.50 (s, 18H), 2.33 (t,2H), 2.42-2.70 (m, 2H), 2.78-3.10 (dd, 2H), 4.18 (q, 3H), 6.88 (s, 1H). LC MS for C21H30N2O7S for [M+H]⁺ calc.455, found 455.

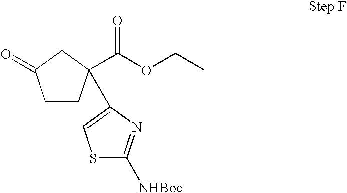

The slow-eluted component from the flash chromatography in Step E, Example 195 was proved to be the title compound (gummy material, 1.80 g). ¹H NMR (300 MHz, CDCl₃): 1.16 (t, 3H), 1.46 (s, 9H), 2.27 (3, 2H), 2.38-2.62 (m,2H), 2.64-3.00 (dd, 2H), 4.11 (q, 2H), 6.66 (s, 1H). LC MS for C16H22N2O5S for [M+H]⁺ calc.355, found 355.

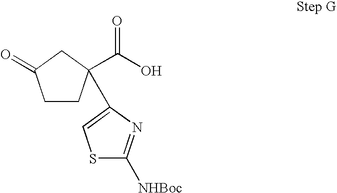

A mixture of 1.4 g (4.0 mmol) of ethyl 1-(2-tert-butoxycarbonyl-amino-thiazol-4-yl)-3-oxo-cyclopentanecarboxylate (Step F, Example 195) and 0.82 g (13 mmol) of lithium hydroxide monohydrate in a solution of 20 mL of MeOH and 2 mL of water was stirred at room temperature overnight. The entire mixture was poured onto a silica gel column and eluted with 10% MeOH/CH₂Cl₂. Evaporation in vacuo afforded a light yellow solid. 1.30 g of the title product was obtained as a fluffy solid. ¹H NMR (300 MHz, CDCl₃): 1.52 (t, 9H), 2.10-3.20 (m, 8H), 6.60 (s, 1H).

### Intermediate 4

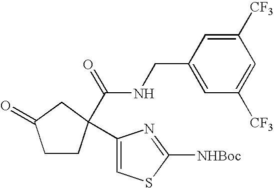

A mixture of 0.65 g (2.0 mmol) of 1-(2-tert-butoxycarbonyl-amino-thiazol-4-yl)-3-oxo-cyclopentane carboxylic acid (Intermediate 3), 0.70 g (2.5 mmol) of (3,5-bis-trifluoromethyl)benzylamine hydrochloride and 0.95 g EDC (5.0 mmol) in 50 mL of CH₂Cl₂ was stirred for 2 h. The reaction mixture was diluted with 100 mL of CH₂Cl₂ and washed with 3 N aqueous HCl (3×50 mL), saturated aqueous NaHCO₃ (50 mL), and water (100 mL) and dried over Na₂SO₄ and evaporated in vacuo. 1.0 g of the title compound was obtained as a yellow solid. ¹H NMR (400 MHz, CDCl₃): 1.55 (s, 9H), 2.10-2.22 (m, 2H), 2.38-2.64 (m, 2H), 2.70-3.23 (dd,2H), 4.48-4.64 (m, 2H), 6.74 (s, 1H), 7.36 (broad, 1H), 7.63 (s, 2H), 7.77 (s, 1H), 7.98 (broad, 1H). LC MS for C23H23F6N3O4S for [M+H]⁺ calc.552, found 552.

### Intermediate 5

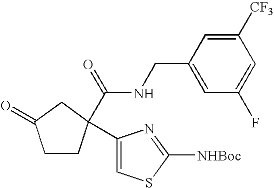

A mixture of 0.65 g (2.0 mmol) of 1-(2-tert-butoxycarbonyl-amino-thiazol-4-yl)-3-oxo-cyclopentanecarboxylic acid (Intermediate 3), 0.65 g (2.5 mmol) of 3-fluoro-5-trifluoromethylbenzylamine hydrochloride and 0.95 g EDC (5.0 mmol) in 50 mL of CH₂Cl₂ was stirred for 2 h. The reaction mixture was diluted with 100 mL of CH₂Cl₂ and washed with 3 N aqueous HCl (3×50 mL), saturated aqueous NaHCO₃ (50 mL), water (100 mL), dried over Na₂SO₄ and evaporated in vacuo. 0.9 g of the title compound was obtained as a yellow solid. ¹H NMR (400 MHz, CDCl₃): 1.56 (s, 9H), 2.18 (m, 1H), 2.38-2.65 (m, 3H), 2.70(d, 1H), 3.12 (d, 1H), 4.48 (m, 2H), 6.74 (s, 1H), 7.10 (d, 1H), 7.20-7.35 (m, 3H), 7.99 (broad, 1H). LC MS for C22H23F4N3O4S for [M+H]⁺ calc.502, found 502.

### Intermediate 6

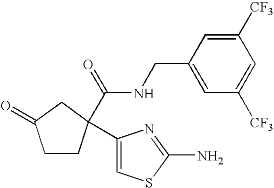

A mixture of 1.1 g (2.0 mmol) of N-(3,5-Bis-trifluoromethyl-benzyl)-1-(2-tert-butoxycarbonyl-amino-thiazol-4-yl)-3-oxo-cyclopentanecarbamide (Intermediate 5) and 5 mL of neat TFA was stirred at room temperature for 1 h and evaporated. The residue was dissolved in 50 mL of EtOAc, washed with saturated aqueous sodium bicarbonate, dried over Na₂SO₄, evaporated and dried under vacuum. The title compound (0.85 g, 94%) was obtained as a yellow solid. ¹H NMR (400 MHz, CDCl₃): 2.20 (m, 1H), 2.38 (m, 1H), 2.52 (m, 2H), 2.60(d, 1H), 3.18 (d, 1H), 4.58 (m, 2H), 5.34 (broad, 2H), 6.31 (s, 1H), 7.65 (2, 2H), 7.75 (s, 1H), 7.80 (broad, 1H). LC MS for C18H15F6N3O2S for [M+H]⁺ calc.452, found 452.

### Intermediate 7

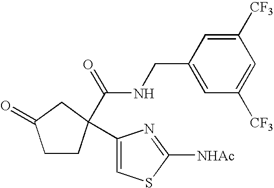

A mixture of 0.85 g (1.9 mmol) of N-(3,5-bis-trifluoromethyl-benzyl)-1-(2-amino-thiazol-4-yl)-3-oxo-cyclopentanecarbamide (Intermediate 6), 1.0 mL of acetic anhydride and 2.0 mL of pyridine in 20 mL of CH₂Cl₂ was stirred overnight, diluted with 50 mL of CH₂Cl₂, washed with water and 2 N aqueous HCl, dried over Na₂SO₄ and evaporated. The title compound (0.74 g) was obtained as a light yellow solid after purification on prep TLC (10% MeOH/CH₂Cl₂). LC MS for C20H17F6N3O3S for [M+H]⁺ calc.494, found 494.

### Intermediate 8

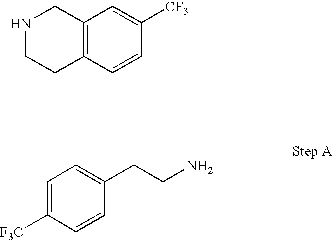

To a solution of 4-trifluoromethyl phenylacetonitrile (40 g, 215 mmol) in 2N NH3/MeOH (400 mL) was added Raney Ni (˜4.0 g). The reaction mixture was placed in a par-shaker and shook under 50 Lb pressure overnight. The solution was filtered through celite and concentrated in vacuo to yield the desired amine (38 g, 95%). ESI-MS calc. For C9H10F3N: 189; Found: 190 (M+M).

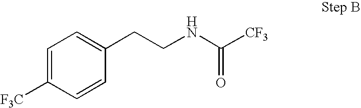

The above amine (Step A, Intermediate 8) (38 g, 200 mmol) and DIEA (52 mL, 300 mmol) were dissolved in DCM (300 mL). The solution was cooled to 0° C. before TFAA (36 mL, 250 mmol) was added slowly. The reaction mixture was stirred in the ice bath for another 10 minutes before warmed up to room temperature. The reaction was completed in 30 minutes and dumped in water and extracted with DCM (2×). The organic layer was washed with 1N HCl and saturated NaCl solution, dried over MgSO₄, and concentrated in vacuo to yield the desired amide (56 g, 98%). ESI-MS calc. For C11H9F6NO: 285; Found: 286 (M+H).

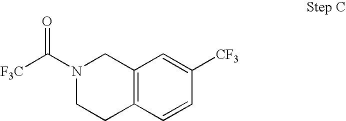

To a mixture of the amide (Step B, Intermediate 8) (73 g, 256 mmol) and paraformaldehyde (11.5 g, 385 mmol) was added 200 mL of acetic acid. The reaction mixture was stirred at room temperature for 5 min before concentrated sulfuric acid (200 mL). An exothermic reaction was observed. After 30 min, TLC showed a complete conversion. The mixture was cooled to RT before poured onto ice water (2000 mL) and extracted with EtOAc (3×500 mL). Combined organic layers were washed with water (2×), saturated NaHCO₃, and brine, dried over MgSO₄, filtered, evaporated and dried in vacuum. The desired amide (72.7 g, 96%) was obtained as a light-yellow solid. 1H NMR (400 MHz, CDCl3) δ 7.22 (q, J=11.67 Hz, 8.46 Hz, 1H), 7.11 (t, J=10.53 Hz, 1H), 7.03 (d, J=11.67 Hz, 1H), 4.79 (d, J=23.57 Hz, 2H), 3.91 (t, J=6.18 Hz, 1H), 3.87 (t, J=5.72 Hz, 1H), 2.97 (m, 2H). ESI-MS calc. For C12H9F6NO: 297; Found: 298 (M+H).

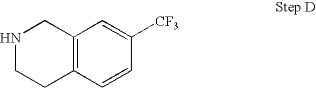

The amide (Step C, Intermediate 8) (50 g, 168 mmol) was dissolved in EtOH (200 mL) before solid K₂CO₃ (50 g, 360 mmol) and H₂O (50 mL) were added. The reaction mixture was refluxed for 15 hours before concentrated in vacuo. The concentrate was diluted with H₂O (100 mL) and extracted with DCM (5×). Combined organic layers were dried over MgSO₄, filtered, concentrated and purified on FC (10% [aq. NH4OH/MeOH 1/9]/DCM) to yield the amine (Step D, Intermediate 8)(30 g, 89%). 1H NMR (400 MHz, CDCl₃) δ 7.11 (d, J=8.4 Hz, 1H), 7.01 (bd, J=8.4 Hz, 1H), 6.89 (s, 1H), 4.03 (s, 2H), 3.15 (t, J=6.1 Hz, 2H), 2.80 (t, J=5.6 Hz, 2H), 1.80 (s, 1H). ESI-MS calc. For C10H10F3N: 201; Found: 202 (M+H).

### Intermediate 9

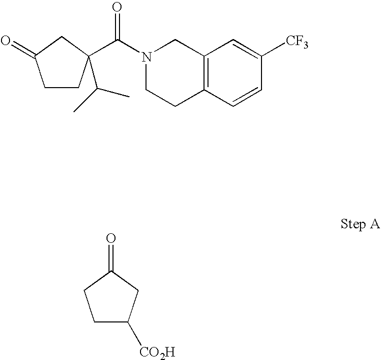

This compound was prepared according to the literature procedure) Stetter, H., Kuhlman, H. Liebigs Ann. Chim., 1979, 944).

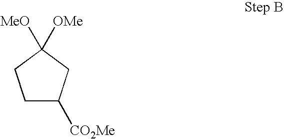

The keto acid (Step A, Intermediate 2) (20 g, 156 mmol) was dissolved in MeOH first before TMOF (85 mL, 781 mmol) was added. TsOH (3 g, 15.6 mmol) was added last. The reaction mixture was stirred at room temperature for 4 hours before concentrated under house vacuum, diluted with ether, quenched with saturated NaHCO₃, washed with brine, and dried over anhydrous MgSO₄. The crude product was purified by flash chromatography (25/75, ether/pentane) to yield the ketal ester (21.52 g, 73.2%). 1H NMR (500 MHz, CDCl₃) δ 3.68 (s, 3H), 3.21 (d, J=9.9 Hz, 6H), 2.89 (p, J=8.5 Hz, 1H), 2.14-2.05 (m, 2H), 2.02-1.80 (m, 4H).

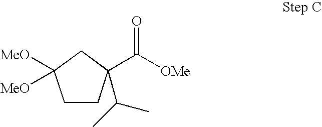

To a flame-dried 500 mL round-bottomed flask, was added dry THF (150 mL). The solution was cooled to −78° C. before iPr₂NH (19.2 mL, 137.3 mmol), 2.5 M nBuLi (55 mL, 137.3 mmol), and neat ketal ester (Step B, Intermediate 2) (21.52 g, 114.4 mmol), were added sequentially. The reaction mixture was stirred at −78° C. for 30 minutes before 2-iodopropane (34.3 mL, 343.2 mmol) was added. After the reaction was stirred for another 20 minutes at −78° C., the mixture was placed in the refrigerator (0° C.) overnight. The mixture was quenched with 10% citric acid and extracted with ether (3×). Combined organic layer was washed with H₂O and brine, dried over anhydrous MgSO₄, and concentrated. The crude product was purified by flash chromatography (20/80 ether/pentane) to yield the alkylated ester (16.74 g, 63.6%). 1H NMR (400 MHz, CDCl3) δ 3.69 (s, 3H), 3.18 (d, J=20.5 Hz, 6H), 2.57 (d, J=13.9 Hz, 1H), 2.29 (m, 1H), 1.90 (p, J=6.8 Hz, 1H), 1.81 (m, 2H), 1.65 (m, 2H), 0.89 (q, J=11.9 Hz, 6.8 Hz, 6H).

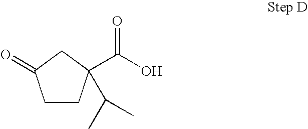

The alkylated ester (Step C, Intermediate 2) (16.74 g, 72.7 mmol) was dissolved in EtOH (30 mL) before a solution of NaOH (11 g, 275 mmol) in H₂O (30 mL) was added. The reaction mixture was refluxed for 3 days before cooled to room temperature and acidified with concentrated HCl. The organic solvent was evaporated under vacuum and the aqueous layer was extracted with DCM (5×). Combined organic layer was dried over anhydrous MgSO₄ and concentrated in vacuo to yield the desired keto acid (11.07 g, 89.5%). %). 1H NMR (500 MHz, CDCl₃) δ 2.70 (d, J=18.1 Hz, 1H), 2.44-2.39 (m, 1H), 2.30-2.15 (m, 2H), 2.14 (dd, J=18.1 Hz, 1.0 Hz, 1H), 2.06 (p, J=6.9 Hz, 1H), 1.98 (m, 1H), 0.98 (dd, J=11.4 Hz, 6.9 Hz, 6H).

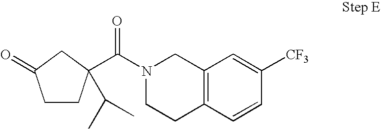

To a solution of the keto acid (Step D, Intermediate 2) (2 g, 11.76 mmol) in DCM (50 mL) was added oxalyl chloride (1.54 mL, 17.64 mmol) followed by 2 drops of DMF. The mixture was stirred at room temperature for 80 minutes before concentrated in vacuo. The concentrate was dissolved in DCM and added slowly to a solution of Intermediate 8 (2.36 g, 11.76 mmol) and Et₃N (2.13 mL, 15.29 mmol) in DCM. The resulting mixture was stirred at room temperature for 18 hours before washed with H₂O, 1N HCl, saturated NaHCO₃, and brine, dried over anhydrous MgSO₄, and concentrated in vacuo. The crude product was purified by MPLC (60/40, EtOAc/Hexanes) to yield 2-E (3.18 g, 76.6%). 1H NMR (500 MHz, CDCl₃) δ 7.46 (d, J=7.3 Hz, 1H), 7.39 (s, 1H), 7.29 (d, J=7.7 Hz, 1H), 4.81 (ABq, 2H), 3.93 (m, 1H), 3.82 (m, 1H), 2.94 (m, 3H), 2.54 (m, 1H), 2.43 (d, J=8.5 Hz, 1H), 2.32 (m, 2 H), 2.26 (p, J=6.6 Hz, 1H), 2.16 (m, 1H), 0.93 (dd, J=19.7 Hz, 6.8 Hz, 6H). LC-MS for C₁₉H₂₃F₃NO₂ [M⁺H⁺] calculated 354.16, found 354.25.

### Intermediate 10

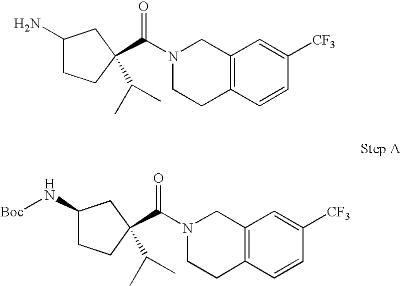

To a flask was added Boc-amino acid (Intermediate 1, 1.10 g, 4 mmol), isoquinoline hydrochloride (Intermediate 8, 0.944 g, 4 mmol), PyBrOP (1.85 g, 4 mmol), DMAP (0.29 g, 2.4 mmol), DIEA (2.77 mL, 16 mmol) and DCM (20 mL). The resulting mixture was stirred for 36 h under nitrogen. The entire material was dumped onto a silica gel column and eluted with 20% EtOAc/Hexane. The desired Boc-amide was obtained as a gummy solid (1.5 g, 82%). ESI-MS calc. for C24H33F3N2O3: 454; Found: 455 (M+H).

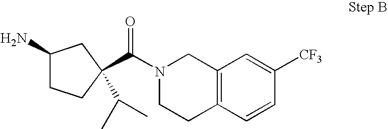

The Boc amino amide (Step A, Intermediate 10) was treated with 10 mL of 4N HCl/Dioxane for 1 h, evaporated, dried in vacuum. The intermediate 11 was obtained as a yellow solid (1.2 g). ESI-MS calc. for C19H25F3N2O: 354; Found: 355 (M+H).

### Intermediate 11

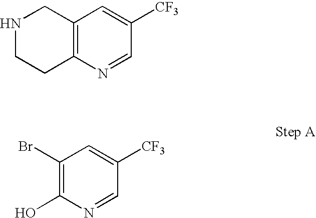

To a solution of 5-trifluoromethyl-2-pyridinal (51 g, 310 mmol) and sodium acetate (26.2 g, 319 mmol) in glacial acetic acid (200 mL) was added bromine (16.7 mL, 325 mmol) and the resulting mixture was heated at 80° C. for 2.5 h. The reaction was allowed to cool to room temperature and then was evaporated under reduced pressure. The residue was neutralized with saturated NaHCO₃ solution and extracted with ethyl acetate (3×200 mL). The organics were combined, dried over MgSO₄, filtered, and evaporated in vacuo to yield 74.45 g (98%) of the crude product. ¹H NMR (400 MHz, CDCl₃) δ 8.04 (d, J=2.6 Hz, 1H), 7.89 (m, 1H).

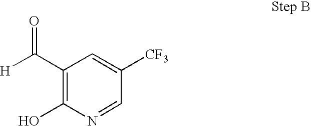

Under nitrogen, the substituted pyridine described in Step A, Intermediate 11 (48.8 g, 202 mmol) was added in small portions to a suspension of NaH (8.9 g, 220 mmol) in anhydrous tetrahydrofuran (500 mL). After complete addition of the intermediate, the reaction mixture was cooled to −78° C. and treated with tert-butyllithium (260 mL, 444 mmol) added dropwise via syringe. After stirring for 5 min, N,N-dimethylformamide (50 mL, 707 mmol) was added slowly to maintain the temperature below −50° C. The resulting mixture was then stirred for 10 h allowing it to warm to room temperature. The mixture was quenched with 2 N HCl and then diluted with ethyl acetate (1000 mL). The organic layer was separated, washed with brine, dried over MgSO4, and evaporated in vacuo. The desired product was precipitated out of ethyl acetate and hexanes and filtered to yield a light brown solid (28.55 g, 74%). ¹H NMR (500 MHz, CD₃OD) δ 10.13 (s, 1H), 8.21 (s, 2H).

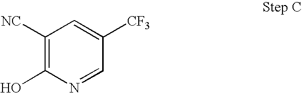

A mixture of the intermediate from Step B, Intermediate 11 (18 g, 95 mmol), sodium formate (7.1 g, 110 mmol), hydroxylamine hydrochloride (7.3 g, 110 mmol), and formic acid (150 mL) was stirred at room temperature for 2 h and then heated to reflux overnight. The reaction mixture was cooled and allowed to stand at room temperature for 7 days. The reaction was poured into water and extracted with ethyl acetate (3×). The combined organic layers were washed with water (2×), saturated NaHCO₃ and brine, dried over Na₂SO₄, filtered, and concentrated in vacuo to yield the desired product as a brown powder (17.84 g, 90%). ¹H NMR (400 MHz, CD₃OD) δ 8.37 (d, J=2.7 Hz, 1H), 8.19 (q, J=0.7 Hz, 0.3 Hz, 1H).

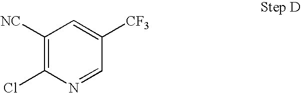

To a mixture of phosphorous oxychloride (13.4 mL, 144 mmol) and quinoline (8.7 mL, 73 mmol) was added the product from Step C, Intermediate 11, (24.6 g, 131 mmol) and the resulting mixture was heated to reflux for 3 h. The reaction was cooled to 100° C. before water (70 mL) was slowly added. The mixture was further cooled to room temperature and neutralized carefully with saturated NaHCO₃ solution. The aqueous layer was extracted with ethyl acetate (3×) and the organic layers were combined, dried over MgSO₄, filtered, and evaporated in vacuo. The crude product was purified by flash chromatography to afford (23.5 g, 87%) of the desired compound. ¹H NMR (500 MHz, CDCl₃) δ 8.88 (d, J=2.0 Hz, 1H), 8.26 (d, J=2.5 Hz, 1H).

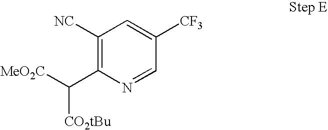

To a suspension of NaH (7.8 g, 200 mmol) in tetrahydrofuran (100 mL) under nitrogen was added dropwise a solution of tert-butyl methyl malonate (20 mL, 120 mmol) in anhydrous tetrahydrofuran (100 mL) via syringe. The reaction mixture was stirred for 0.5 h before a solution of the intermediate prepared in Step D, Intermediate 11 (20.1 g, 97.6 mmol) in tetrahydrofuran (200 mL) was added slowly via syringe. The reaction was stirred at room temperature overnight, then quenched with a saturated solution of NH₄Cl. The organic layer was separated and the aqueous layer was extracted with ethyl acetate (3×). The combined organic layers were washed with water (3×), dried over Na₂SO₄, filtered, and evaporated in vacuo. Flash chromatography afforded 31.76 g (95%) of the pure desired product. ¹H NMR (500 MHz, CDCl₃) δ 9.03 (d, J=1.5 Hz, 1H), 8.25 (d, J=2.0 Hz, 1H), 5.25 (s, 1H), 3.86 (s, 3H), 1.52 (s, 9H).

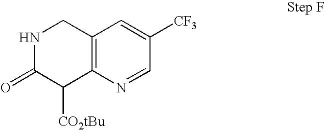

A suspension of Raney Ni (1 g) and the product from Step E, Intermediate 11 (18.2 g, 52.9 mmol) in ethanol (130 mL) was placed on a Parr Apparatus and hydrogenated at 40 psi H₂ overnight. The suspension was filtered through celite and the filtrate was evaporated in vacuo to afford 16.35 g (98%) of the crude product. ¹H NMR (500 MHz, CDCl₃) δ 8.83 (s, 1H), 7.89 (s, 1H), 7.82 (s, 1H), 4.83 (d, J=16 Hz, 1H), 4.72 (s, 1H), 4.49 (d, J=16 Hz, 1H), 1.45 (s, 9H).

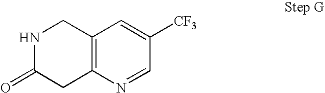

To the mixture of the product from Step F, Intermediate 11 (16 g, 51 mmol) in dichloromethane (60 mL) was added TFA (30 mL) and the resulting mixture was stirred at room temperature for 0.5 h. The solution was evaporated under reduced pressure and the residue was dissolved in dichloromethane. The mixture was neutralized by the slow addition of a solution of saturated sodium bicarbonate and the organic layer was removed. The aqueous layer was extracted with dichloromethane (4×) and the combined organic layers were dried over Na₂SO₄, filtered, and evaporated in vacuo to afford 10.42 g (95%) of the desired product. ¹H NMR (400 MHz, CDCl₃) δ 8.81 (s, 1H), 7.78 (s, 1H), 7.30 (s, 1H), 4.63 (s, 2H), 3.90 (s, 2H).

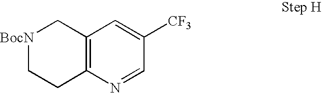

To a solution of the product from Step G, Intermediate 11 (18.0 g, 83.3 mmol) in tetrahydrofuran (50 mL) was added 1.0 M borane in tetrahydrofuran (417 mL, 420 mmol) and the resulting solution was stirred at room temperature overnight. The solution was evaporated under reduced pressure and the residue was treated with 1% HCl/methanol solution. The resulting mixture was heated at 50° C. overnight to breakdown the borane complex. Treatment with acidic methanol was repeated twice to insure that the borane complex was removed. A solution of this crude product (83.3 mmol, assuming 100% conversion) and diisopropylethylamine (43 mL, 250 mmol) in dichloromethane was treated with di-tert-butyl dicarbonate (36.4 g, 167 mmol) and the resulting mixture was stirred at room temperature overnight. The solution was washed with saturated sodium bicarbonate solution, water, and brine. The aqueous layers were combined and back-washed with dichloromethane (2×). The combined organic layers were then dried over Na₂SO₄, filtered, and evaporated to dryness. The crude product was purified by flash chromatography and MPLC to afford (11.89 g, 47%) as a yellow solid. ¹H NMR (500 MHz, CDCl₃) δ 8.69 (s, 1H), 7.66 (s, 1H), 4.67 (s, 2H), 3.79 (t, J=6.0 Hz, 2H), 3.08 (t, J=5.5 Hz, 2H), 1.51 (s, 9H).

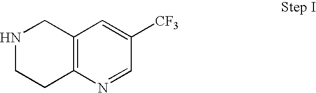

The product described in Step H, Intermediate 11 (11.89 g) was treated with a solution of 4 N HCl in dioxane. The solution was stirred at room temperature for 2 h and then evaporated in vacuo to afford Intermediate 12 (10.85 g, 99%) as a yellow powder. LC-MS for C₉H₁₀F₃N₂ calculated 202.07, found [M+H]⁺ 203.0.

### Intermediate 12

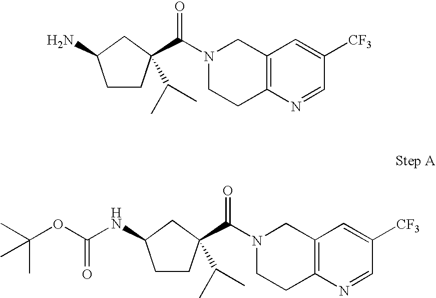

Intermediate 11 (4.6 g, 16 mmol) and Intermediate 1 (4.0 g, 14 mmol) were first dried by azeotropic distillation with toluene (3×50 mL) and placed under high vacuum for 30 min. Under nitrogen, 4-dimethylaminopyridine (1.08 g, 8.60 mmol), anhydrous dichloromethane (40 mL), and diisopropylethylamine (7.0 mL, 40 mmol) were added sequentially. After Intermediate 8 was in solution, bromo-tris-pyrrolidino-phosphonium hexafluorophosphate (6.80 g, 14.3 mmol) was added, immediately followed by additional diisopropylethylamine (7.0 mL, 40 mmol). The reaction mixture was stirred at room temperature overnight and then quenched with saturated NaHCO₃. The aqueous layer was back washed with dichloromethane (3×50 mL) and the organic layers were combined, dried over Na₂SO₄, filtered, and evaporated in vacuo. The crude product was purified by flash chromatography (stepwise gradient 0-60%, ethyl acetate/hexanes) to afford the product (4.80 g, 74%) as a yellow foam. ¹H NMR (500 MHz, CDCL₃) δ 8.72 (s, 1H), 7.70 (s, 1H), 4.88 (br d, J=17.0 Hz, 1H), 4.78 (d, J=17.6 Hz, 1H), 4.04-3.84 (m, 2 H), 3.52 (br s, 1H), 3.12 (br t, J=5.6 Hz, 1H), 2.32-2.06 (m, 3H), 1.98-1.70 (m, 4H), 1.64-1.54 (m, 1H), 1.44 (s, 9H), 0.92-0.82 (m, 6H). LC-MS for C₂₃H₃₂F₃N₃O₃ calculated 455.24, found [M+H]⁺456.2.

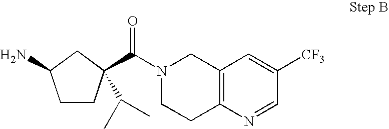

The from Step B, Intermediate 12 (1.2 g, 2.6 mmol) was dissolved with 4 N HCl in dioxane (50 mL) and the resulting solution was stirred at room temperature for 1 h. The reaction was evaporated under vacuum to afford the product (904 mg, 97%) as a white powder. LC-MS calculated for C₁₈H₂₄F₃N₃O is 355.20, found [M+H]⁺356.2.

### Intermediate 13

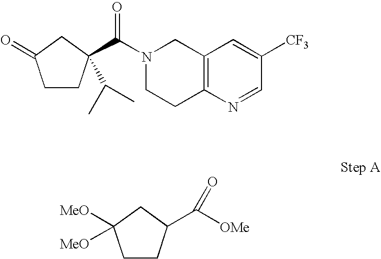

A solution of methyl-3-oxocyclopentane-carboxylate (20 g, 160 mmol) and trimethylorthoformate (85 mL, 780 mmol) in methanol was treated with a catalytic amount of p-toluenesulfonic acid (3 g, 15.6 mmol) and the resulting solution was stirred for 4 h at room temperature. The solvent was evaporated under reduced pressure and the residue was then dissolved in ether (600 mL). The solution was washed with saturated sodium bicarbonate (2×200 mL), water (150 mL), brine (200 mL), dried over anhydrous sodium sulfate, filtered, and the solvent evaporated as before. Purification by flash column (eluant: 25% ether/pentane) afforded 21.52 g (73%) of the desired product as a clear oil. ¹H NMR (500 MHz, CDCl₃) δ 3.68 (s, 3H), 3.21 (d, J=9.9 Hz, 6H), 2.89 (p, J=8.5 Hz, 1H), 2.14-2.05 (m, 2H), 2.02-1.80 (m, 4H).

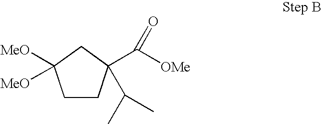

A flame dried 500 mL round bottom flask was charged with 150 mL of dry tetrahydrofuran, and then, set under nitrogen and cooled to −78° C. using an acetone/dry ice bath. Diisopropylamine (19.2 mL, 137 mmol) was added to the cooled solvent via syringe. 2.5 M n-butyllithium in hexanes (55 mL, 140 mmol) was slowly added to the solution. After 5 min stirring, the methyl ketal described in Step A, Intermediate 3 (21.52 g, 114.4 mmol) in 50 mL of tetrahydrofuran was added dropwise via syringe and the resulting mixture was stirred at −78° C. for 2 h. 2-iodopropane (34.3 mL, 343 mmol) was then added dropwise via syringe and the resulting mixture was stirred overnight allowing it to warm slowly to room temperature. The reaction was quenched with a solution of 10% citric acid and the organics were separated. The aqueous layer was extracted with ether (3×150 mL) and all the organics were combined, dried over anhydrous sodium sulfate, filtered, and evaporated under reduced pressure. The crude product was purified by flash column using an eluant of 20% ether/pentane to afford 16.74 g (64%) of the desired product. ¹H NMR (400 MHz, CDCl₃) d 3.69 (s, 3H), 3.18 (d, J=20.5 Hz, 6H), 2.57 (d, J=13.9 Hz, 1H), 2.29-2.20 (m, 1H), 1.90 (p, J=6.8 Hz, 1H), 1.88-1.80 (m, 2H), 1.69-1.61 (m, 2H), 0.89 (dd, J=11.9 Hz, 6.8 Hz, 6H).

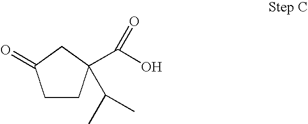

A solution of the ester from Step B, Intermediate 13 (16.74 g, 72.7 mmol) in ethanol (30 mL) was treated with 5 M aqueous NaOH (55 mL) and the resulting mixture was heated to reflux for 3 days. The mixture was then cooled to room temperature and acidified with concentrated hydrochloric acid. The organic solvent was evaporated under reduced pressure and the aqueous layer was then extracted with dichloromethane (5×100 mL). The organic extracts were combined, dried over anhydrous magnesium sulfate, filtered, and evaporated in vacuo to yield the crude 3-oxocyclopentane carboxylic acid (11.07 g, 90%) as a yellow oil. Purification was not attempted because of the compounds polarity and lack of a chromophore. ¹H NMR (500 MHz, CDCl₃) d 2.70 (d, J=18.1 Hz, 1H), 2.44-2.39 (m, 1H), 2.30-2.15 (m, 2H), 2.14 (dd, J=18.1, 1.0 Hz, 1H), 2.06 (p, J=6.9 Hz, 1H), 1.98 (m, 1H), 0.98 (dd, J=11.4, 6.9 Hz, 6H).

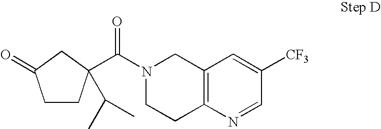

To a solution of the acid from Step C (540 mg, 3.20 mmol) in dichloromethane (50 mL) was added oxalyl chloride (0.834 mL, 9.60 mmol) followed by 2 drops of N,N-dimethylformamide. The solution was stirred at room temperature for 80 min and then evaporated under reduced pressure. The residue was dissolved in dichloromethane (2 mL) and added via syringe to a prepared solution of Intermediate 12 (880 mg, 3.20 mmol) and triethylamine (0.820 mL, 6.50 mmol) in dichloromethane (20 mL). The resulting mixture was stirred at room temperature for 18 h and then quenched with water (25 mL). The organics were separated, washed with saturated sodium bicarbonate and brine, dried over anhydrous sodium sulfate, filtered, and evaporated. The crude product was purified by MPLC using a step-wise gradient eluant of 0-70% ethyl acetate/hexanes to afford Intermediate 2 (720 mg, 64%). ¹H NMR (500 MHz, CDCl₃).

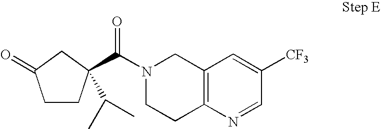

Resolution of product from Step D, Intermediate 13 was accomplished by chiral separation using an HPLC equipped with a preparative ChiralPak AD column. The separation was accomplished by injecting 100 mg/run and using an eluant of 25% isopropanol and 75% heptane with a flow rate of 9 mL/min.

### Example 15

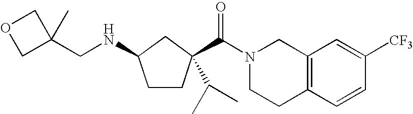

A solution of Intermediate 9 (0.130 g, 0.37 mmol) in DCM (2.0 ml) at RT under N₂ atmosphere, in the presence of 4 Å molecular sieve, was treated with 0.074 g (0.73 mmol) of 1-(3-methyloxetan-3-yl)methenamine dissolved in DCM (2.0 ml) and the resulting mixture stirred for 45 min. 0.154 g (0.73 mmol) Na(AcO)₃BH was then added to the flask and the resulting mixture stirred over night. The mixture was filtered through celite and the filtrated conc. in vacuo. The cis and trans products (racemic) were separated by prep plate TLC eluting with 30% EtOAc/10% MeOH/1% NH₄OH/hexane. The racemic cis diastereomers were then separated by reverse phase chiral HPLC on an OD column, eluting with 20% isopropanol/heptane. 0.0152 g of the title product (band 1) and 0.0146 g (band 2) of it's cis diastereoisomer were obtained. LC MS for C₂₄H₃₃F₃N₂O₂ [M+H]⁺calc. 439.25, found 439.2.

### Example 16

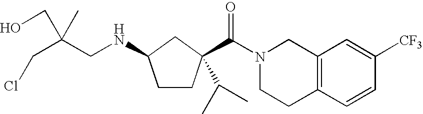

Example 15, upon treatment with 2.0 N HCl (2.0 ml) afforded the title product as the HCl salt. LC MS for C₂₄H₃₄ ClF₃N₂O₂ [M+H]⁺ calc. 475.23, found 475.1.

### Example 17

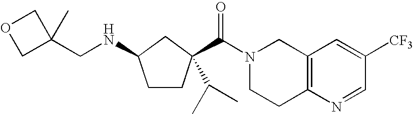

Following the procedure for Example 15 but using Intermediate 13 instead of Intermediate 9 afforded the title product. LC MS for C₂₃H₃₂F₃N₃O₂ [M+H]⁺ calc. 440.24, found 440.2.

### Example 18

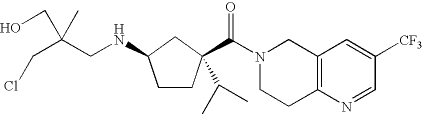

Example 17, upon treatment with 2.0 N HCl (2.0 ml), afforded the title product as the HCl salt. LC MS for C₂₃H₃₃ ClF₃N₃O₂ [M+H]⁺ calc. 476.22, found 476.2.

### Example 19

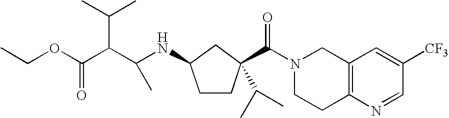

A solution of Intermediate 12 (0.1 g, 0.23 mmol, HCl salt) in DCM (3.0 ml) under N₂ atmosphere in the presence of 4 Å molecular sieves, was treated with 0.08 mL (0.46 mmol) of N,N-diisopropylethylamine and 0.08 g (0.46 mmol) ethyl-α-isopropylacetoacetate. After stirring for 45 min at room temperature 0.146 g (0.69 mmol) Na(AcO)₃BH was added to the mixture and stirred over night. The mixture was filtered through celite and the filtrate was concentrated in vacuo. Reverse phase HPLC afforded 54 mg (46%) of the title product as a mixture of 4 diastereoisomers. LC MS for C₂₇H₄₀F₃N₃O₃ [M+H]⁺ calc. 512.30, found 512.2.

### Examples 20-38

Following the procedure described in Example 19, a series of analogous target compounds were synthesized. Their structure and MS-characteristics are summarized in the following Table.

TABLE 2

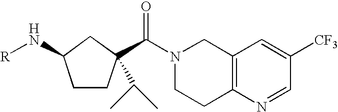

| | | Molecular | Calc'd | Found |
|---|---|---|---|---|
| Ex. | R | Formula | [M + H]⁺ | [M + H]⁺ |
| 20 | 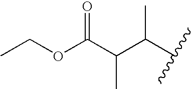 | C₂₅H₃₆F₃N₃O₃ | 484.27 | 484.3 |
| 21 | 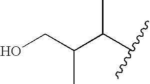 | C₂₃H₃₄F₃N₃O₂ | 442.26 | 442.3 |
| 22 | 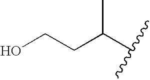 | C₂₂H₃₂F₃N₃O₂ | 428.24 | 428.2 |
| 23 | 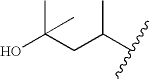 | C₂₄H₃₆F₃N₃O₂ | 456.28 | 456.2 |
| 24 | 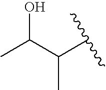 | C₂₂H₃₂F₃N₃O₂ | 428.24 | 428.2 |
| 25 | 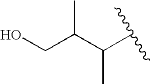 | C₂₃H₃₄F₃N₃O₂ | 442.26 | 442.2 |
| 26 | 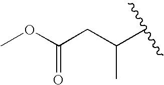 | C₂₃H₃₂F₃N₃O₃ | 456.24 | 456.2 |
| 27 | 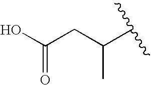 | C₂₂H₃₀F₃N₃O₃ | 442.22 | 442.2 |
| 28 | 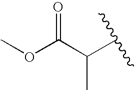 | C₂₂H₃₀F₃N₃O₃ | 442.22 | 442.1 |
| 29 | 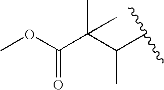 | C₂₅H₃₆F₃N₃O₃ | 484.27 | 484.3 |
| 30 | 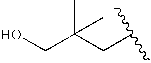 | C₂₃H₃₄F₃N₃O₂ | 442.26 | 442.2 |
| 31 | 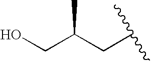 | C₂₂H₃₂F₃N₃O₂ | 428.50 | 428.2 |
| 32 | 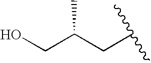 | C₂₂H₃₂F₃N₃O₂ | 428.50 | 428.2 |
| 33 | 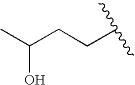 | C₂₂H₃₂F₃N₃O₂ | 428.50 | 428.2 |
| 34 | 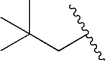 | C₂₃H₃₄F₃N₃O | 426.27 | 426.2 |
| 35 | 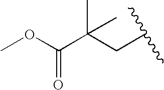 | C₂₄H₃₄F₃N₃O₃ | 470.26 | 470.2 |
| 36 | 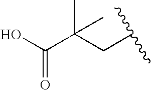 | C₂₃H₃₂F₃N₃O₃ | 456.24 | 456.2 |
| 37 | 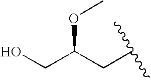 | C₂₂H₃₂F₃N₃O₃ | 444.24 | 444.2 |
| 38 | 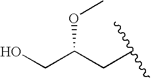 | C₂₂H₃₂F₃N₃O₃ | 444.24 | 444.2 |

### Example 39

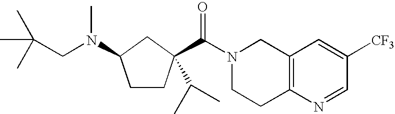

To a solution of Example 34 (0.045 g, 0.105 mmol) in anhydrous MeOH (3.0 mL) was added 0.079 mL (1.057 mmol, 37% in water) formaldehyde and 0.02 g (0.317 mmol) NaBH₃CN and the mixture stirred for 18 h. The solvent was evaporated and the resulting oil diluted with water/DCM. The layers were separated and the organic layer dried (MgSO₄) and concentrated in vacuo. Reverse phase HPLC afforded the title product which was converted into the HCl salt. LC MS for C₂₄H₃₆F₃N₃O [M+H]⁺ calc. 440.28, found 440.2.

### Intermediate 14

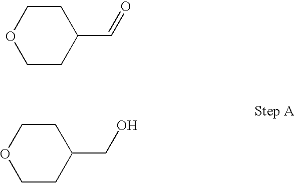

A solution of methyl tetrahydro-2H-pyran-4-carboxylate (2.0 g, 14 mmol) in anhydrous THF (20 mL) at 0° C. under N₂ was treated with 14 mL (14 mmol, 1.0 M in THF) LAH and the mixture stirred for 2 h. The reaction was quenched with 0.5 ml of water followed by 0.5 mL 15% NaOH and finally another 1.5 mL water. The resulting white suspension was filtered through celite and the filtrate dried (MgSO₄) and concentrated in vacuo to afford the title product (1.1 g) as a clear oil. ¹H NMR (CDCl₃, 400 MHz) δ: 3.99-4.03, (dd, 2H), 3.51-3.53 (d, 2H), 3.38-3.45 (t, 2H), 1.72-1.80 (m, 1H), 1.65-1.68 (d, 1H), 1.52 (s (b),1H), 1.29-1.40 (m, 2H).

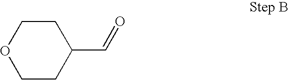

A solution of oxalyl chloride (4.73 mL, 9.47 mmol, 2.0 M in DCM) at −78° C. under a N₂ atmosphere was treated with 1.34 mL (18.9 mmol) DMSO slowly and after 5 min the product from Step A, dissolved in DCM, was added slowly. The resulting mixture was stirred for 15 min and 6.25 mL triethylamine added to the mixture. After 5 min the reaction was stirred at rt for 1 h and quenched with water. The layers were separated and the aqueous layer washed (×2) DCM, the combined organic layers was dried (MgSO₄) and conc. in vacuo. The title compound was afforded as an oil and used in the next step without further purification. ¹H NMR (CDCl₃, 400 MHz) δ: 9.62 (s, 1H), 4.15-4.17 (d, 2H), 3.45-3.55 (t, 2H), 3.15-3.18 (m, 2H), 1.99-2.15 (m, 1H), 1.82-1.89 (d, 2H).

### Intermediate 15

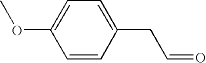

To a solution of 4-allylanisole (1.0 g, 6.7 mmol) in THF/H₂O (1:1) 40 mL, was added 17.1 mg OsO₄ followed by 4.28 g (20.1 mmol) NaIO₄. After 1 h stirring the mixture was diluted with ether and the layers separated. The organic layer was dried (MgSO₄) and conc. in vacuo to afford the title product. ¹H NMR (CDCl₃, 400 MHz) δ: 9.74 (s, 1H), 7.04-7.14 (d, 2H), 6.91-6.94 (d, 2H), 3.82 (s, 3H), 3.65 (s, 2H).

### Example 40

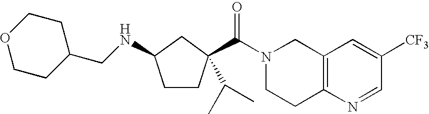

Following the procedure for Example 19 with Intermediate 14, the title product was obtained by reverse phase HPLC purification. LC MS for C₂₄H₃₄F₃N₃O₂ [M+H]⁺ calc. 454.26, found 454.15.

### Example 41

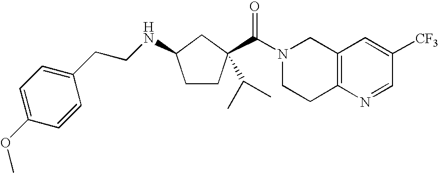

Following the procedure for Example 19 with Intermediate 15, the title product was obtained by reverse phase HPLC purification. LC MS for C₂₇H₃₄F₃N₃O₂ [M+H]⁺ calc. 490.26, found 490.2.

### Example 42

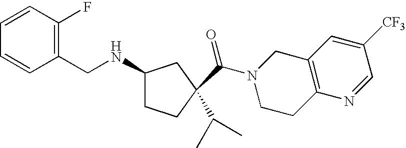

To a stirred suspension of Intermediate 12 (hydrochloride, 50 mg, 0.117 mmol) in 2 mL of methylene chloride at room temperature was added 40 μL (0.3 mmol) of triethylamine and 14.4 mg (0.1166 mmol) of 2-fluorobenzaldehyde. To the reaction mixture was added 4 beads of molecular sieves 4 A followed by 50 mg of NaOAc)₃BH. After stirring overnight the reaction mixture was evaporated and the product (48 mg) was isolated by Gilson reverse phase chromatography C2F₄N₃O [M+H]⁺ calc. 463.52, found 505.

### Examples 43 to 57

Following the procedure described in Example 42, a series of analogous target compounds were synthesized. Their structure and MS-characteristics are summarized in the following Table.

TABLE 3

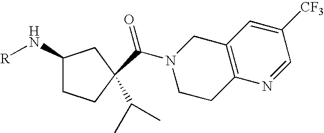

| | | Molecular | Calc'd | Found |
|---|---|---|---|---|
| Ex. | R | Formula | [M + H]⁺ | [M + H]⁺ |
| 43 | 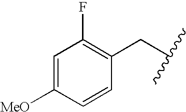 | C₂₆H₃₁F₄N₃O₂ | 494.54 | 494.2 |
| 44 | 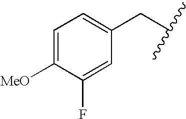 | C₂₆H₃₁F₄N₃O₂ | 494.54 | 494.2 |
| 45 | 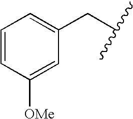 | C₂₆H₃₂F₆N₃O₂ | 476.55 | 476.2 |
| 46 | 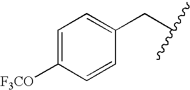 | C₂₆H₂₉F₉N₂O | 530.53 | 530.2 |
| 47 | 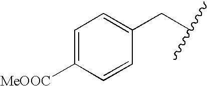 | C₂₇H₃₂F₃N₃O₃ | 504.57 | 504.2 |
| 48 | 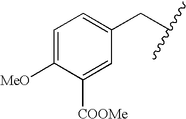 | C₂₈H₃₄F₃N₃O₄ | 534.59 | 534.5 |
| 49 | 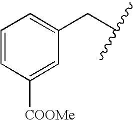 | C₂₇H₃₂F₃N₃O₃ | 504.57 | 504.2 |
| 50 | 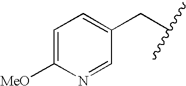 | C₂₅H₃₁F₃N₃O₂ | 4772.4 | 477.2 |
| 51 | 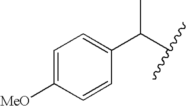 | C₂₇H₃₄F₃N₃O₂ | 490.26 | 490.3 |
| 52 | 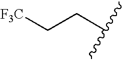 | C₂₁H₂₇F₆N₃O | 452.45 | 452.2 |
| 53 | 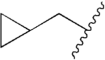 | C₂₁H₂₈F₃N₃O | 396.47 | 396.2 |
| 54 | 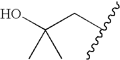 | C₂₂H₃₄F₃N₃O₂ | 442.54 | 442.2 |
| 55 | 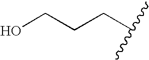 | C₂₁H₃₀F₃N₃O₂ | 414.48 | 414.2 |
| 56 | 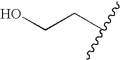 | C₂₀H₂₈F₆N₃O₂ | 400.46 | 400.1 |
| 57 | 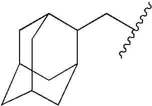 | C₂₅H₂₉F₄N₃₀ | 504.65 | 504.2 |

### Example 58

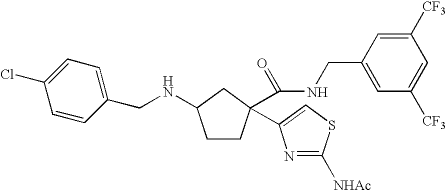

To a stirred solution of Intermediate 7 (100 mg, 0.203 mmol) in DCM (2 mL) was added 4-chlorobenzyl amine (74.1 mg, 0.528 mmol), molecular sieve (4 Å, excess, not measured) and Na(OAc)₃BH (172 mg, 0.812 mmol). The resulting reaction mixture was stirred at room temperature overnight before being filtered and purified by preperative TLC using 10% MeOH in DCM as an eluting solvent. The two diastereomers were separated: High band −58A, and low band −58B). The products were converted to the desired HCl salts by adding 4 N HCl (50 μL). Both compounds were confirmed by LC-MASS. Calc. MW=618 for C₂₇H₂₅N₄O₂SClF₆, found M+1=619.

### Example 59 and Example 60

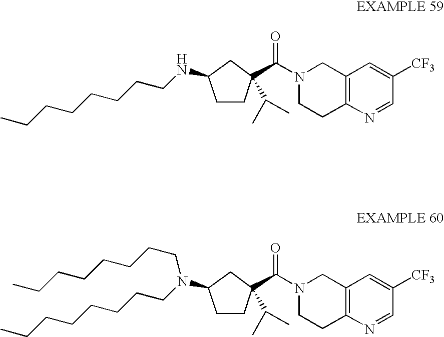

Intermediate 12 (45 mg, 0.11 mmol) was combined with octanal (17 mg, 0.13 mmol), DIEA (38 μL, 0.22 mmol), sodium triacetoxyborohydride (110 mg, 0.55 mmol), and 4 Å molecular sieves (50 mg) in DCM (10 mL). The resulting reaction mixture was stirred at room temperature for 2 days before being diluted with DCM and washed with aqueous saturated sodium bicarbonate and brine. The organic layer was dried over Na₂SO₄, filtered, and concentrated under reduced pressure. The products were separated and purified by preparative TLC (2.7% MeOH/0.7% NH₄OH/97% DCM) and converted to their HCl salts by the addition of 2 M HCl in ether. After concentration, 11 mg of Example 59 was obtained along with 15 mg Example 60.

EXAMPLE 59: LC-MS calculated for C₂₆H₄₀F₃N₃O: Exact Mass: 467.31; Found 468.4.

EXAMPLE 60: LC-MS calculated for C₃₄H₅₆F₃N₃O: Exact Mass: 579.44; Found 580.45.

### Example 61

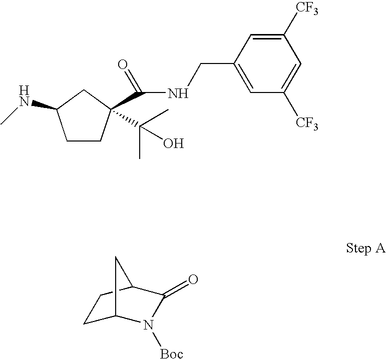

50 g (0.46 mol) of (1S,4R)-(+)-2-azabicyclo[2.2.1]hept-5-en-3-one in 200 mL of methanol containing 2.5 g of Pd/C (10%) was hydrogenated on a Parr Apparatus under 50 psi of hydrogen for 1 h. The catalyst was removed by filtration through a pad of celite. The filtrates were evaporated and the residue was dried in vacuum. The resulting white solid (50 g) was dissolved in 200 mL of methylene chloride and 110 g (0.50 mol) of di-tert-butyl dicarbonate and 1.0 g of DMAP were added. The reaction mixture was stirred at room temperature overnight and then loaded on a silica gel column, eluted with 10% EtOAc/Hexane. The title compound (83 g, 86%) was obtained as a white solid. ¹NMR (400 MHz, CDCl₃): 1.40 (d, 1H), 1.51 (s,9H), 1.70-1.95 (m, 5H), 2.84 (m, 1H), 4.50 (m, 1H).

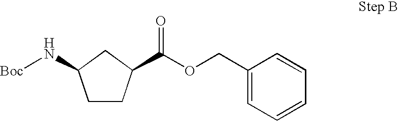

To a stirred mixture of 63.0 g (300 mmol) of (1S,4R)-(+)-N-BOC-2-azabicyclo[2.2.1]hept-3-one and 32 g (300 mmol) of benzyl alcohol in 200 mL of THF under nitrogen was added 2.8 g (300 mmol) of lithium hydride in multiple portions. The resulting mixture was stirred overnight. TLC showed a complete conversion. The entire mixture was poured into a stirred mixture of ice-water/EtOAc (500 mL). The organic phase was separated and washed with water (2×200 mL), dried over Na₂SO₄, evaporated and Dried in vacuum. The title compound (95.5 g, 100%) was obtained as a white solid. ¹H NMR (400 MHz, CDCl₃): 1.44 (s, 9H), 1.60 (m, 1H), 1.72 (m, 1H), 1.95 (m, 3H), 2.24 (m, 1H), 2.90 (m, 1H), 4.08 (m, 1H), 4.98 (broad, 1H), 5.13 (s, 2H), 7.38 (m, 5H).

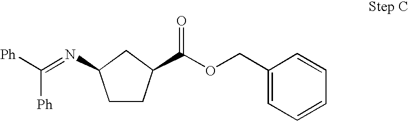

A mixture of 96 g (300 mmol) of (1S,3R)-benzyl-(N-BOC-3-amino)-cyclopentanecarboxylate and 300 mL of 4N HCl in dioxane was stirred for 1 h. The solvent was removed under reduced pressure, and the residue was dried under high vacuum overnight and then suspended in 300 mL of CH₂Cl₂. To this suspension was added 54.4 g of benzophenone imine. The resulting mixture was stirred overnight. The precipitate was removed by filtration and the filtrates were washed with brine, dried over Na₂SO₄, evaporated and dried in vacuum. The title compound was obtained as a light yellow oil (116.0 g, 100%). ¹H NMR (400 MHz, CDCl₃): 1.80 (m, 1H), 1.95 (m, 2H), 2.15 (m, 2H), 2.50 (m, 1H), 2.89 (m, 1H), 3.61 (m, 1H), 5.20 (s, 2H), 7.18 (d, 2H), 7.38 (m, 8H), 7.47 (m, 3H), 7.64 (d, 2H).

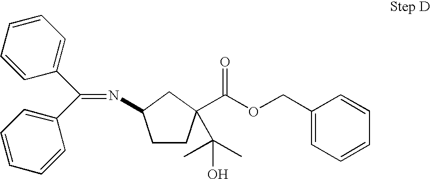

To a flame-dried 500 mL round-bottomed flask, was added dry THF (130 mL). The solvent was cooled to −78° C. before diisopropylamine (10.5 mL, 75.2 mmol), 2.5 M n-butyllithium (30 mL, 75 mmol), and a solution of the product prepared in Step C (25 g, 65 mmol) in THF (20 mL), were added sequentially. The reaction mixture was stirred at −78° C. for 30 minutes before acetone (14.4 mL, 196 mmol) was added. After the reaction was stirred for another h, the mixture was quenched with saturated NH₄Cl, extracted with ether, dried over MgSO4, and concentrated. The crude product was purified by MPLC (EtOAc:Hexanes/25:75). Cis and trans isomers were resolved with cis being the desired isomer (cis, 6.8 g; trans, 3.47 g). %). Cis isomer: ¹H NMR (400 MHz, CDCl₃): 7.58 (m, 2H), 7.48-7.28 (m, 1H), 7.14 (m, 2H), 5.22 (s, 2H), 3.78 (p, J=12.1 Hz, 6.2 Hz, 1H), 3.46 (s, 1H), 2.56-2.50 (m, 1H), 2.27 (dd, J=13.9 Hz, 5.9 Hz, 1H), 2.08 (dd, J=13.8 Hz, 6.6 Hz, 1H), 1.92 (m, 1H), 1.83-1.69 (m, 2H), 1.09 (d, J=14.0 Hz, 6H).

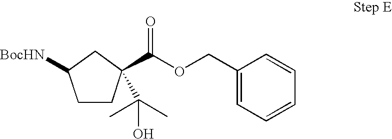

The imine from previous step (6.8 g, 15 mmol) was dissolved in THF (50 mL) before 2 N aqueous HCl (50 mL) was added. The reaction mixture was stirred and monitored by TLC. After completion of reaction, the mixture was concentrated in vacuo to remove THF. The aqueous layer was basisified to pH 9.0 with saturated Na₂CO₃ solution and extracted with DCM. The organic layer was dried over MgSO₄ and di-tert-butyl dicarbonate (4.4 g, 20 mmol) was added. The reaction was stirred at room temperature overnight before being extracted with DCM, dried over MgSO₄, and concentrated in vacuo. The crude product was purified by column chromatography to yield (2.9 g, 50%). ¹H NMR (400 MHz, CDCl₃) 7.39 (m, 5H), 5.20 (s, 2H), 4.62 (bs, 1H), 4.13 (b, 1H), 3.40 (s, 1H), 2.25 (dd, J=14.5 Hz, 8.1 Hz, 1H), 2.16 (m, 1H), 2.01 (m, 2H), 1.89 (m, 1H), 1.44 (s, 9H), 1.18 (s, 6H).

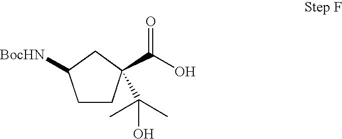

A mixture of the benzyl ester form previous step (2.9 g), Pd/C (300 mg), and ethanol (50 mL) were placed on a Parr Apparatus under 50 psi pressure overnight. The mixture was filtered through celite and concentrated in vacuo to yield the desired product (2.01 g, 91.0%). ¹H NMR (500 MHz, CDCl₃): 6.56 (s, ½ H), 5.17 (s, ½ H), 4.00 (d, J=43.3 Hz, 1H), 2.40-1.70 (m, 6H), 1.46 (b, 9H), 1.27 (b, 6H).

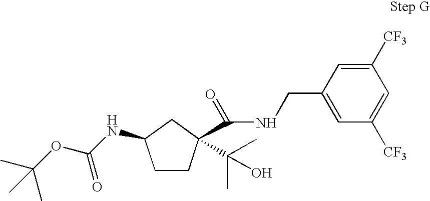

A solution of the acid from Step F (3.72 g, 13.0 mmol), 3,5-bistrifluoromethylbenzylamine hydrochloride (3.62 g, 13.0 mmol), diisopropylethylamine (2.26 mL, 13.0 mmol), 1-hydrozy-7-azabenzotriazole (1.76 g, 13.0 mmol) in dichloromethane (30 mL) was treated with EDC (3.72 g, 19.4 mmol) and the reaction mixture was stirred at room temperature for 2 h. It was poured onto water (50 mL) and extracted with dichloromethane. The combined organic extracts were, washed with brine, dried with anhydrous, magnesium sulfate and the solvent was removed in vacuo to leave 4.80 g of an oily crude product. This was further purified by column chromatography (Silica gel, ethyl acetate hexanes/2:3) to yield 3.18 g (48%) of the pure product. ¹H NMR (500 MHz, CDCl₃): 8.40 (bs, 1H), 7.76 (s, 1H), 7.75 (s, 2H), 5.34 (d, J=6.18 Hz, 1H), 4.56 (m, 2H), 4.0 (m, 1H), 3.21 (s, 1H), 2.15 (dd, J=14.2, 4.81 Hz, 1H), 2.05 to 1.85 (m, 4H), 1.62 (m, 1H), 1.41 (bs, 9H), 1.26 (s, 3H), 1.23 (s, 3H). ¹³C NMR (125 MHz, CDCl₃): 178.4, 155.7, 141.8, 131.9 (m), 127.5, 121.0, 79.1, 74.6, 52.3, 42.7, 37.8, 33.4, 31.6, 28.3, 27.0, 26.3.

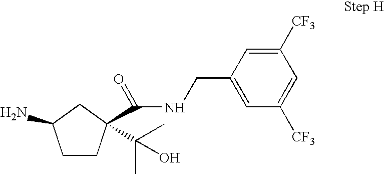

The solution of the BOC-protected amine from the previous step (3.18 g, 6.20 mmol) was stirred at room temperature in dioxane/HCl (4.0 N) for 1 h. The solvent was removed in vacuo to yield the pure hydrochloride (2.63 g, 94%). LC MS for C₁₈H₂₂F₆N₂O₂ for [M+H]⁺ calc. 413.16, found 413.20.

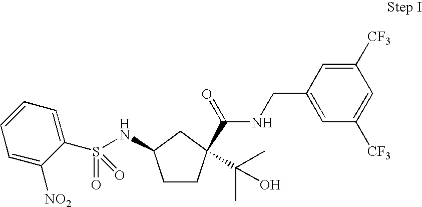

A solution of the amine hydrochloride from the previous step (147 mg, 0.328 mmol), diisopropylethylamine (228 μL, 1.31 mmol) in dichloromethane (8 mL) was treated with 2-nitrophenylsulfonyl chloride (88 mg, 0.39 mmol) and stirred at room temperature for 1 h. The reaction mixture was diluted with dichloromethane (50 mL) and washed with water (2×50 mL). The organic layer was dried with anhydrous sodium sulfate, filtered, and the solvent was removed in vacuo. The residue (188 mg) was purified by preparative TLC (ethylacetate:hexanes/6:4) to afford 145 mg of the desired product. ¹H NMR (500 MHz, CDCl₃): 8.23 (bt, J=5.72 Hz, 1H), 8.12 (m, 1H), 7.75 (bm, 5H), 6.59 (d, J=7.78 Hz), 4.63 (dd, J=16.0, 6.0 Hz), 4.53 (dd, J=15.6, 6.0 Hz), 4.0 (m, 1H), 2.45 (s, 1H), 2.22 (dd, J=14.4, 2.5 Hz, 1H), 2.02 (dd, J=13.7, 6.7 Hz, 1H), 1.80 (m, 3H), 1.6 (m, 1H), 1.24 (s, 3H), 1.18 (s, 3H). LC MS for C₂₄H₂₅F₆N₃O₆S for [M+H]⁺ calc. 598.14, found 598.15.

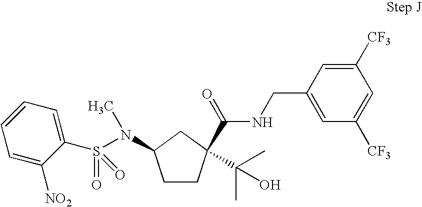

A solution of tributylphosphine ((200 μL, 0.804 mmol) in THF (8 mL), was cooled to 0° C. and neat diethyl azodicarboxylate (126 μL, 0.804 mmol) was added via syringe. After stirring at cold for 30 minutes, a solution of the amide from the previous step (240 mg, 0.402 mmol) and methyl alcohol (100 mL, 2.47 mmol) in THF (6 mL) was added. The cooling bath was removed, and the reaction mixture was stirred at ambient temperature for 2 h. The solvent was evaporated to dryness, the residue was diluted with water (20 mL), and the crude product was extracted with ethyl acetate (3×30 mL). The combined organic extracts were dried with anhydrous magnesium sulfate, filtered, and the solvent was removed in vacuo. The residue (459 mg) was further pre-purified by preparative TLC (ethyl acetate+hexanes/6:4), and purified again using preparative TLC and benzene-diethyl ether (4:1) as an eluent. In this fasion, 126 mg (63%) of the desired pure product was obtained. ¹H NMR (500 MHz, CDCl₃): 8.07 (t, J=6.0 Hz, 1H), 7.97 (m, 1H), 7.75-7.60 (m, 6 H), 4.57 (d, J=6.0 Hz, 2H), 4.30 (m, 1H), 4.18 (m, 2H), 3.18 (s, 1H), 2.82 (s, 3H), 2.64 (s, 1H), 2.32 (m, 1H), 2.14 (m, 1H), 1.92 (dd, J=14.0, 8.9 Hz, 1H), 1.70 (m, 3H), 1.25 (m, 9H). LC MS for C₂₅H₂₇F₆N₃O₆S for [M+H]⁺ calc. 612.15, found 612.10.

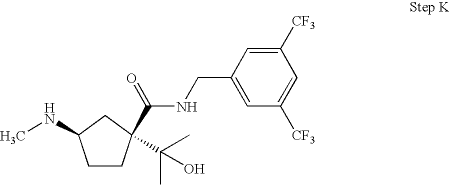

The mixture of the sulfonamide, preparation of which was described in the previous step (145 mg, 0.237 mmol), potassium carbonate (flame dried, 100 mg, 0.279 mmol) and DMF was treated with thiophenol (30 μL, 0.24 mmol) and stirred at room temperature for 6 h. The reaction was quenched by pouring onto water (5 mL) and the product was extracted with ethyl acetate (3×10 mL). The combined organic extracts were dried (anhydrous sodium sulfate), filtered, and the solvent was evaporated to dryness to yield 151 mg of crude product. This was further purified by preparative TLC (ethyl acetate+ethyl alcohol+ammonium hydroxide/90:9:1) to afford 37 mg (37%) of the pure product. ¹H NMR (500 MHz, CDCl₃): 10.30 (s, 1H), 7.79 (s, 1H), 7.74 (s, 2H), 5.82 (s, 1H), 4.57 (dd, J=15.3, 5.5 Hz, 1H), 4.44 (dd, J=15.3, 5.5 Hz, 1H), 3.25 (m, 1H), 2.35 (m, 1H), 2.26 (s, 3H), 1.95 (bm, 3H), 1.70 (bm, 4H), 1.32 (s, 3H), 1.17 (s, 3H). LC MS for C₁₉H₂₄F₆N₂O₂ for [M+H]⁺ 427.40, found 427.15.

### Examples 62 to 96

The following table shows other compounds that where prepared as described in Example 1, using different aldehydes. The products were confirmed by LC-MS in each case.

TABLE 4

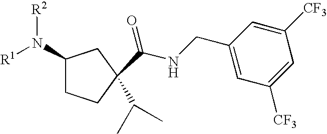

| | | | Molecular | Calc'd | Found |
|---|---|---|---|---|---|
| Ex. | R¹ | R² | Formula | [M + H]⁺ | [M + H]⁺ |
| 62 | 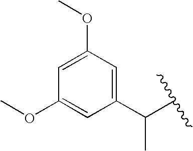 | H | C₂₈H₃₄F₆N₂O₃ | 561 | 561 |
| 63 | 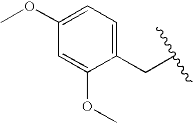 | H | C₂₇H₃₂F₆N₂O₃ | 547 | 547 |
| 64 | 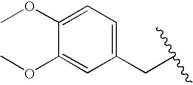 | H | C₂₇H₃₂F₆N₂O₃ | 547 | 547 |
| 65 | 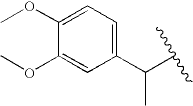 | H | C₂₈H₃₄F₆N₂O₃ | 561 | 561 |
| 66 | 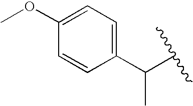 | H | C₂₇H₃₂F₆N₂O₂ | 531 | 531 |
| 67 | 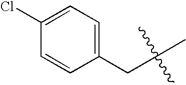 | H | C₂₅H₂₇ClF₆N₂O | 521 | 521 |
| 68 | 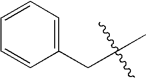 | 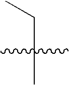 | C₂₇H₃₂F₆N₂O | 515 | 515 |
| 69 | 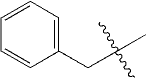 | 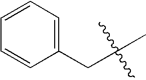 | C₃₂H₃₄F₆N₂O | 577 | 577 |
| 70 | 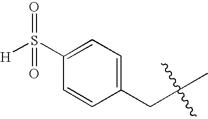 | H | C₂₅H₂₈F₆N₂O₃S | 551 | 551 |
| 71 | 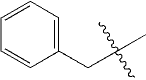 | 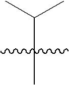 | C₂₈H₃₄F₆N₂O | 529 | 529 |
| 72 | 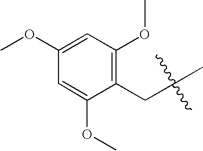 | H | C₂₈H₃₄F₆N₂O₄ | 577 | 577 |
| 73 | 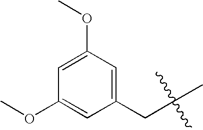 | H | C₂₇H₃₂F₆N₂O₃ | 547 | 547 |
| 74 | 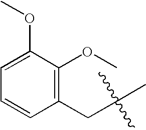 | H | C₂₇H₃₂F₆N₂O₃ | 547 | 547 |
| 75 | 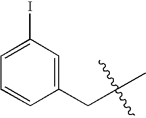 | H | C₂₅H₂₇F₆IN₂O | 613 | 613 |
| 76 | 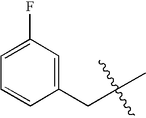 | H | C₂₅H₂₇F₇N₂O | 505 | 505 |
| 77 | 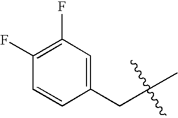 | H | C₂₅H₂₆F₈N₂O | 523 | 523 |
| 78 | 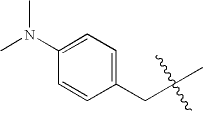 | H | C₂₇H₃₃F₆N₃O | 530 | 530 |
| 79 | 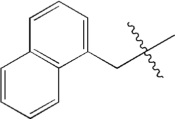 | H | C₂₉H₃₀F₆N₂O | 537 | 537 |
| 80 | 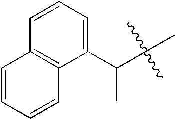 | H | C₃₀H₃₂F₆N₂O | 551 | 551 |
| 81 | 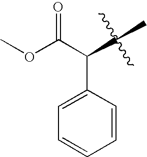 | H | C₂₇H₃₀F₆N₂O₃ | 545 | 545 |
| 82 | 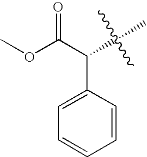 | H | C₂₇H₃₀F₆N₂O₃ | 545 | 545 |
| 83 | 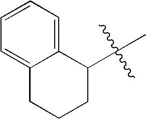 | H | C₂₈H₃₂F₆N₂O | 527 | 527 |
| 84 | 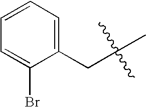 | H | C₂₅H₂₇BrF₆N₂O | 565 | 565 |
| 85 | 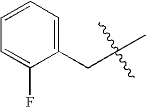 | H | C₂₅H₂₇F₇N₂O | 505 | 505 |
| 86 | 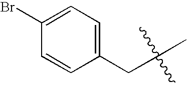 | H | C₂₅H₂₇BrF₆N₂O | 565 | 565 |
| 87 | 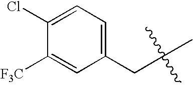 | H | C₂₆H₂₆ClF₉N₂O | 589 | 589 |
| 88 | 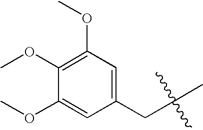 | H | C₂₈H₃₄F₆N₂O₄ | 577 | 577 |
| 89 | 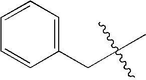 | 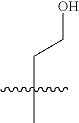 | C₂₇H₃₂F₆N₂O₂ | 531 | 531 |
| 90 | 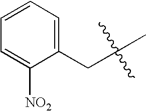 | H | C₂₅H₂₇F₆N₃O₃ | 532 | 532 |
| 91 | 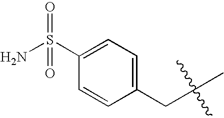 | H | C₂₅H₂₉F₆N₃O₃S | 566 | 566 |
| 92 | 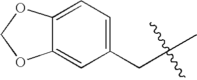 | H | C₂₆H₂₈F₆N₂O₃ | 531 | 531 |
| 93 | 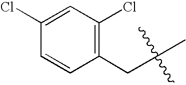 | H | C₂₅H₂₆Cl₂F₆N₂O | 555 | 555 |
| 94 | 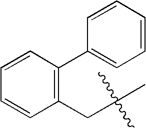 | H | C₃₁H₃₂F₆N₂O | 563 | 563 |
| 95 | 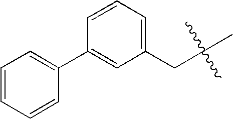 | H | C₃₁H₃₂F₆N₂O | 563 | 563 |
| 96 | 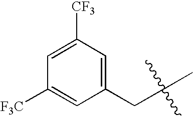 | H | C₂₇H₂₆F₁₂N₂O | 623 | 623 |

### Examples 97 to 106

The following table shows compounds that where prepared as described in Example 15, using different aldehydes. The products were confirmed by LC-MS in each case.

TABLE 5

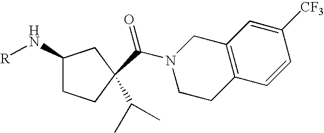

| Ex. | R |
|---|---|
| 97 | 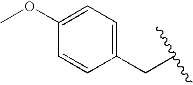 |
| 98 | 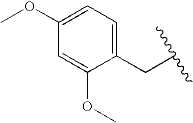 |
| 99 | 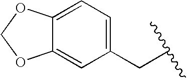 |
| 100 | 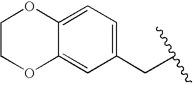 |
| 101 | 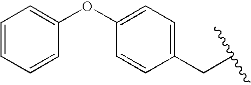 |
| 102 | 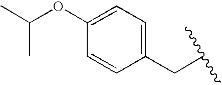 |
| 103 | 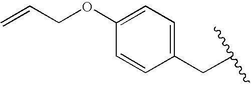 |
| 104 | 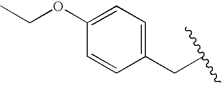 |
| 105 | 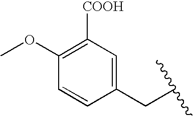 |
| 106 | 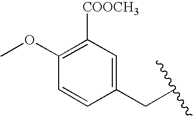 |

### Example 107

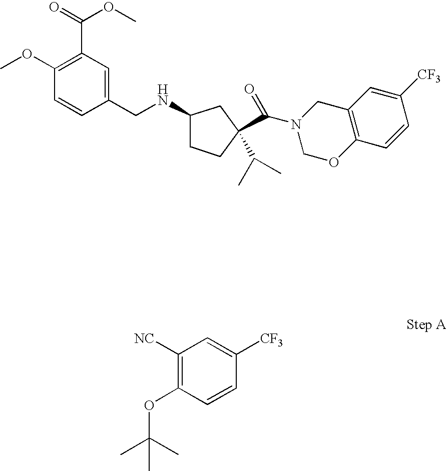

To a cooled (0° C.) solution of 2-fluoro-5-trifluoromethylbenzonitrile (5.23 g, 27.7 mmol) in 140 mL of THF was added, dropwise at a rapid pace, a suspension of potassium t-butoxide (3.88 g, 34.6 mmol) in 35 mL of THF. The reaction mixture was permitted to slowly warm to rt and stirred overnight. The reaction mixture was concentrated under reduced pressure; then ether and 1 M HCl solution were added, and the layers separated. The ethereal layer was washed with saturated NaHCO₃ solution, then brine, dried over anhydrous MgSO₄, filtered, and concentrated. Purification by MPLC (silica, 25% ethyl acetate/hexane) afforded a white crystalline solid. H NMR (CDCl₃, 500 MHz): δ 7.84 (d, J=2.0 Hz, 1H), 7.73 (dd, J=8.5, 2.0 Hz, 1H), 7.27 (d, J=9.0 Hz), 1.55 (s, 9H).

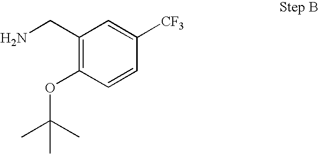

To a solution of the nitrile prepared as described in Step A (7.6 g, 31 mmol) in ethanol (100 mL) was added ammonium hydroxide solution (28-30%, 25 mL) and Raney® 2800 nickel (slurry in water, ˜3.5 g). The resulting mixture was agitated under 50 psi of hydrogen gas for 24 h using a Parr Apparatus. The reaction mixture was then filtered through celite washing with ethanol and then water. The filtrate was concentrated to dryness under reduced pressure, and the residue so obtained was purified by flash chromatography [silica, 5 to 10% gradient (1% increments) of (10% ammonium hydroxide solution (28-30%)/methanol) in DCM] to afford 1-[2-tert-butoxy-5-(trifluoromethyl)phenyl]methanamine as a colorless oil which crystallized upon storage in the freezer. H NMR (CDCl₃, 500 MHz): δ 7.56 (d, J=2.0 Hz, 1H), 7.44 (dd, J=8.5, 2.0 Hz, 1H), 7.12 (d, 8.5 Hz, 1H), 3.90 (s, 2H), 2.70 (br s, 2H), 1.51 (s, 9H).

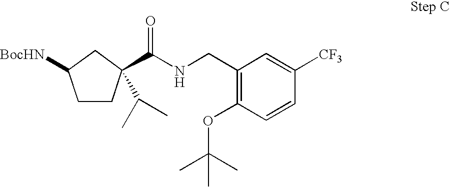

Intermediate 1 (3.42 g, 12.6 mmol) was combined with the product from Step B (3.43 g, 13.9 mmol) and EDC (3.62 g, 18.9 mmol) in DCM (50 mL). After 24 h at room temperature the reaction mixture was treated with 200 mg of DMAP and the resulting solution was stirred for 2 days. The reaction mixture was diluted with DCM and washed with saturated aqueous sodium bicarbonate, 1 N HCl, and then brine. The organic layer was dried over MgSO₄, filtered, and concentrated under reduced pressure to give 3.50 g of the crude desired product which was purified by MPLC (0-50% EA/hexanes) to give 1.64 g of the desired pure product

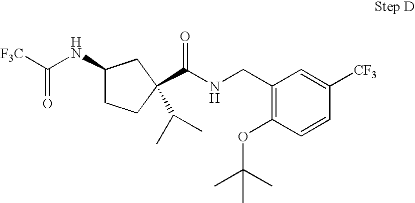

The product form the previous step (1.64 g, 3.28 mmol) was dissolved in 4 M HCl in dioxane (40 mL) and was stirred at room temperature for 3.5 h before being concentrated under reduced pressure and dried under high vacuum overnight. The resulting HCl salt was dissolved in DCM (200 mL) and DIEA (1.1 mL, 6.5 mmol) and treated with TFAA (509 μL, 3.61 mmol). The resulting reaction mixture was stirred at room temperature for 2.5 h before being quenched with bicarb and diluted with DCM. The layers were separated and the DCM layer was washed with 1 N HCl and brine. The organic layer was dried over MgSO4, filtered, and concentrated under reduced pressure to give 1.53 g of the desired product. LC-MS [M+Na]=463.45.

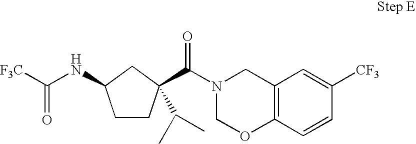

The product from the previous step (1.53 g) was combined with p-toluenesulfonic acid (150 mg) and paraformaldehyde (1.5 g) in benzene (50 mL). The resulting reaction mixture was heated to reflux for 4 h in a Dean/Stark trap assembly. The reaction mixture was cooled to room temperature and was diluted with diethyl ether and washed with saturated aqueous sodium bicarbonate and then brine. The organic layer was dried over MgSO₄, filtered, and concentrated under reduced pressure to give 1.53 g of the desired product. LC-MS (M+H)=453.

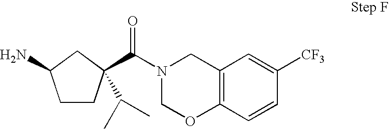

The product from the previous step (1.53 g, 3.38 mmol) was combined with K₂CO₃ (2.23 g, 16.9 mmol) in a mixture of water (2.5 mL) and methanol (100 mL). The resulting reaction mixture was stirred at room temperature overnight, and then was heated to 50° C. for 4.5 h to effect the conversion. The reaction mixture was concentrated and the crude material was diluted with DCM and washed with aqueous saturated sodium bicarbonate and then brine. The organic layer was dried over Na₂SO₄, filtered and concentrated under reduced pressure to give 1.31 g of the desired product which did not require any purification. LC-MS (M+H)=357.

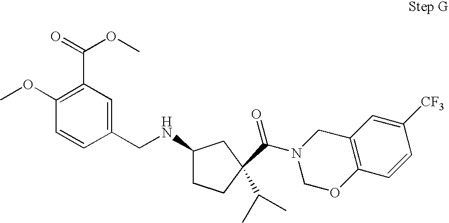

A mixture of amine from step F (0.022 g, 0.047 mmol), aldehyde (9.0 mg, 0.046 mmol) in dichloromethane (2 mL) at room temperature was added 4 Å molecular sieves (0.05 g) followed by Na(OAc)₃BH (50 mg, 0.24 mmol). The resultant mixture was stirred for 12 h and then filtered. The dichloromethane layer was washed with brine, dried, evaporated and purified by preparative chromatography to yield 18 mg of desired compound as hydrochloride salt. LC-MS (M+H)=535.4

### Example 108

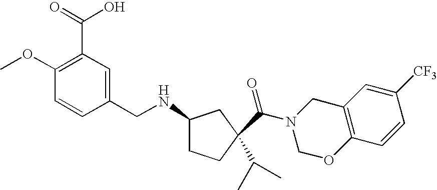

A mixture of Example 107 (15 mg, 0.028 mmol) in THF/MeOH (1.0 mL, 1:1) at room temperature was added lithium hydroxide monohydrate (60 mg, 0.14 mmol) and stirred overnight. The volatiles were evaporated, and purified reverse phase chromatography to yield 12 mg of the desired compound. LC-MS (M+H)=521.5

### Example 109

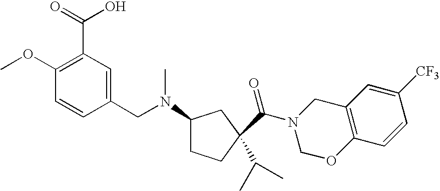

To Example 108 (0.025 g, 0.044 mmol) in MeOH (3.0 mL) was successively added formalin solution (10 equivalents, 37% solution in water) followed by NaCNBH₃ (0.014 g, 0.22 mmol) and the resultant mixture was stirred at room temperature overnight. The reaction mixture was diluted with water and extracted with ethyl acetate. The ethyl acetate layer was washed with brine, dried and evaporated to yield the crude product that was subsequently taken up in THF/MeOH (2.0 mL, 1:1) and saponified with LiOH (5 equivalents) in a procedure analogous to the one described for Example 108. LC-MS (M+H)=535.5

### Example 110

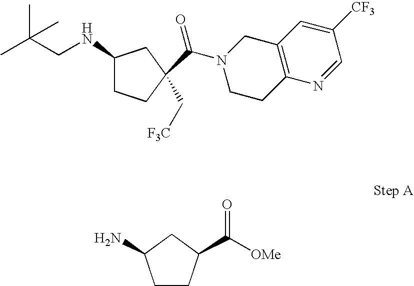

A mixture of (1S)-(+)-2-azabicyclo[2.2.1]hept-5-en-3-one (10.3 g, 94.4 mmol) in ethyl acetate (200 mL) and 10% Pd/C (0.5 g), was hydrogenated at room temperature. After 24 h the reaction mixture was filtered and evaporated leaving behind 10.4 g (100%) of the product that was taken in 250 mL methanol and HCl (12 M, 6 mL). The resultant mixture was stirred at room temperature, until the reaction was complete (72 h). Evaporation of methanol followed by drying under high vacuum, yielded title compound as an off white solid (16.0 g, 96%). ¹H NMR (500 MHz, D₂O): δ 3.70 (s, 3H), 3.01 (m, 1H), 2.38 (m, 1H), 2.16-1.73 (m, 6H).

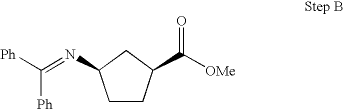

To a suspension of the intermediate from Step A (10.2 g, 56.8 mmol) in dry dichloromethane (200 mL) was added benzophenone imine (10.2 g, 56.8 mmol) at room temperature and the resultant mixture was stirred for 24 h. The reaction mixture was filtered and the filtrate was evaporated, to leave behind a yellow oil that was triturated with ether (100 mL), filtered and evaporated. This operation was repeated twice to ensure that the product was free of ammonium chloride impurities. The resultant oil was thoroughly dried under vacuum to yield the title compound (18.03 g, >100%) and required no further purification. 1H NMR (500 MHz, CDCl₃): δ 7.5-7.18 (m, 10H), 3.75 (m, 1H), 3.7 (s, 3H), 2.78 (m, 1H), 2.26-1.71 (m, 6H).

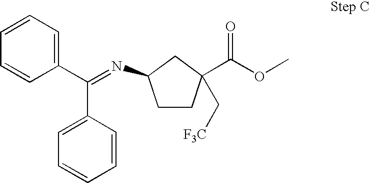

A flame dried 1000 mL round bottom flask was charged with 400 mL of dry tetrahydrofuran, and then, set under nitrogen and cooled to −78° C. using an acetone/dry ice bath. Diisopropylamine (27.4 mL, 195 mmol) was added to the cooled solvent via a syringe. The resulting solution was slowly treated with 2.5 M n-butyllithium in hexanes (55 mL, 140 mmol). After 5 min stirring, the product described in Step B (40 g, 130 mmol) in 100 mL of tetrahydrofuran was added dropwise via syringe and the resulting mixture was stirred at −78° C. for 2 h. 2-iodo-1,1,1-trifluoroethane (47 mL, 480 mmol) was then added dropwise via syringe and the resulting mixture was stirred overnight allowing it to warm slowly to room temperature. The reaction was quenched with a saturated solution of ammonium chloride (400 mL) and the organics were separated. The aqueous layer was extracted with ethyl acetate (3×150 mL) and all the organics were combined, dried over anhydrous sodium sulfate, filtered, and evaporated under reduced pressure. The crude product was used in the next step without further purification. LC-MS for C₂₂H₂₂F₃NO₂ calculated 389.26, found [M+H⁺] 390.4

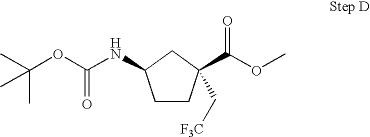

To a solution of the product from Step A, Intermediate 12 (130 mmol, assuming 100% conversion) in 200 mL of tetrahydrofuran was added 200 mL of 2 N hydrochloric acid and the resulting mixture was stirred overnight at room temperature. The solution was concentrate in vacuo to remove the tetrahydrofuran and the aqueous layer was then diluted with dichloromethane (300 mL). The pH of the aqueous layer was adjusted to a pH of 10 by the slow addition of 5 N sodium hydroxide with vigorous stirring. The organic layer was removed using a separatory funnel and the aqueous layer was extracted with dichloromethane (2×150 mL). The organic layers were combined, dried over anhydrous sodium sulfate, and filtered. To the filtrate was added diisopropylethylamine (22.7 mL, 130 mmol) and di-tert-butyl dicarbonate (32.7 g, 150 mmol) and the resulting solution was stirred at room temperature overnight. The mixture was washed with 1 N hydrochloric acid, followed by a saturated solution of sodium bicarbonate, and brine. The organic layer was dried over anhydrous sodium sulfate, filtered, and evaporated under reduced pressure. Purification by MPLC (5 g per run) afforded 5.87 g (14%) of the desired cis (R, S) isomer and 12.31 g (29%) of the undesired trans (S, S) isomer. Also, 5.22 g (12%) was recovered as a 1:1 mixture of the 2 diastereomers. ¹H NMR (500 MHz, CDCl₃) δ (1^{st} desired isomer) 5.05 and 4.40 (singlets, 1H), 3.76 (s, 3H), 2.73 (ddd, J=11.0, 12.8, 14.8 Hz, 1H), 2.38 (ddd, J=10.7, 12.8, 15.0 Hz, 1H) 2.32-2.26 (m, 1H), 2.21 (br dd, J=3.6, 14.5 Hz, 1H), 2.18-2.11 (m, 1H), 2.02 (dd, J=8.8, 14.4 Hz, 1H), 1.61 (dd, J=7.8, 13.2 Hz, 1H) 1.52 (br s, 10H). ¹H NMR (500 MHz, CDCl₃) δ (2^{nd} undesired isomer) 4.52 and 4.06 (singlets, 1H), 3.72 (s, 3H), 2.72 (dd, J=7.1, 13.5 Hz, 1H), 2.66 (ddd, J=10.6, 12.8, 15.0 Hz, 1H), 2.53 (ddd, J=11.0, 12.8, 14.9 Hz, 1H) 2.26 (app dd, J=7.1, 13.5 Hz, 1H), 2.18-2.07 (m, 1H), 1.78 (dd, J=8.6, 13.5 Hz, 1H),1.57-1.48 (m, 2H) 1.46 (s, 9H).

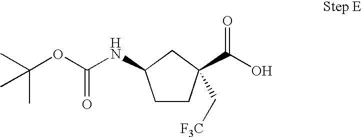

To a mixture of the desired cis (R,S) product described in Step B, Intermediate 12 (4.0 g, 12 mmol) in a 1:1:1 solution of tetrahydrofuran/methanol/water (84 mL) was added solid LiOH (2.60 g, 62.0 mmol) and the resulting solution was heated to 60° C. and stirred for 18 h. The mixture was left standing to cool to room temperature and then concentrated to remove the organic solvent. The aqueous layer was acidified by the slow addition of 6 N hydrochloric acid to pH 4-5. The acidic aqueous layer was extracted with dichloromethane (3×100 mL) and the organics were combined, dried over anhydrous sodium sulfate, filtered, and evaporated under reduced pressure to afford Intermediate 12 (3.86 g, 99%) as a yellow oil. After two days standing at 5° C. in the refrigerator, the material crystallized.

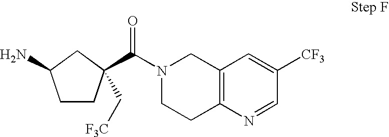

This compound was prepared in an analogous fashion to Intermediate 12, except Intermediate 1 was replaced with the product from Step E. LC-MS for C₁₇H₁₉F₆N₃O calculated 395.17, found [M+H]⁺ 396.2.

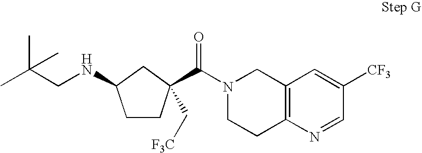

Example 110 was synthesized according to the procedure described for the preparation of Example 19 using the product from Step F and trimethylacetaldehyde. LC-MS for C₂₂H₂₉F₆N₃O calculated 465.22, found [M+H⁺] 466.1.

### Example 111

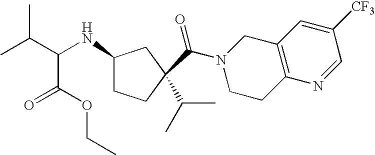

Potassium carbonate (124 mg, 0.9 mmol) was added to a mixture of Intermediate 12 (105 mg, 0.3 mmol) and ethyl 2-bromo-3-methylbutanoate (82 mg, 0.39 mmol) in DMF (5 mL). The reaction was stirred for 40° C. overnight, then poured into ethyl acetate. The organic layer was washed three times with saturated sodium bicarbonate, dried over MgSO₄ and concentrated to yield a residue that was purified by reverse phase HPLC. LC-MS for C₂₅H₃₆F₃N₃O₃ calculated 483.58, found [M+H]⁺ 484.4.

### Example 112

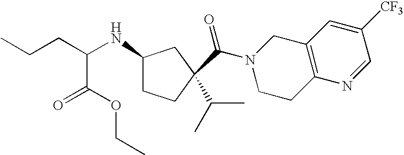

A similar procedure to example 111 was followed using Intermediate 12 and ethyl 2-bromopentanoate. LC-MS for C₂₅H₃₆F₃N₃O₃ calculated 483.58, found [M+H]⁺ 484.5.

### Example 113

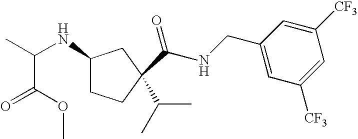

A similar procedure to example 111 was followed using Intermediate 2 and methyl 2-bromopropanoate. LC-MS for C₂₂H₂₈F₆N₂O₃ calculated 482.48, found [M+H]⁺ 483.5.

### Example 114

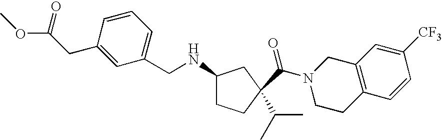

Intermediate 10 (174 mg, 0.5 mmol), methyl-(3-bromomethyl)phenyl acetate (40 mg, 0.2 mmol) and dichloromethane (5 ml) was stirred overnight at room temperature. Reaction mixture was concentrated to give a crude oil which was further purified by reverse phase HPLC to afford the trifluoroacetic acid salt of desired product as a yellow oil (41 mg, 41%). LC-MS for C₂₉H₃₆F₃N₂O₃ calculated 516.61, found [M−CH₃]⁺ 501.13

### Example 115

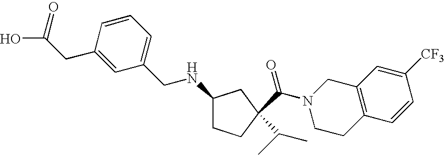

A mixture of example 114 (20 mg, 0.03 mmol), 1.0 N aqueous lithium hydroxide solution (2 ml) and ethanol (2 ml) was stirred at room temperature overnight. Reaction mixture was concentrated to give an oil which was further purified by reverse phase HPLC to afford the acid as a colorless oil (10 mg, 50%). LC-MS for C₂₈H₃₄F₃N₂O₃ calculated 502.58, found M⁺ 503.68

What is claimed:

1. A compound of Formula I: 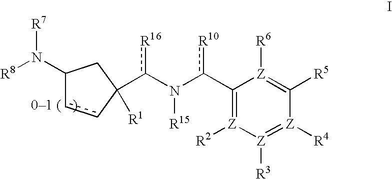
wherein:
Z is N or C, where no more than two Z are N;
R¹ is selected from: —C₁₋₆alkyl, —C₀₋₆alkyl-O—C₁₋₆alkyl, —C₀₋₆alkyl-S—C₁₋₆alkyl, —C₀₋₆alkyl-SO₂-C₁₋₆alkyl, —C₀₋₆alkyl-SO—C₁₋₆alkyl, —C₀₋₆alkyl-SO₂—NR¹²—C₀₋₆alkyl, —(C₀₋₆alkyl)-(C₃₋₇cycloalkyl)-(C₀₋₆alkyl), hydroxy, heterocycle, —CN, —NR¹²R¹², —NR¹²COR¹³, —NR¹²SO₂R¹⁴, —COR¹¹, —CONR¹²R¹², and phenyl;, where alkyl and the cycloalkyl are unsubstituted or substituted with 1-7 substituents independently selected from: halo, hydroxy, —O—C₁₋₃alkyl, trifluoromethyl, C₁₋₃alkyl, —O—C₁₋₃alkyl, —COR¹¹, —SO₂R¹⁴, —NHCOR¹⁵, —NHSO₂CH₃, —heterocycle, ═O, and —CN, and where phenyl and heterocycle are independently unsubstituted or substituted with 1-3 substituents independently selected from: halo, hydroxy, C₁₋₃alkyl, C₁₋₃alkoxy, trifluoromethyl and NHCOR¹⁵;
when the Z attached to R² is N, R² is oxygen or is absent, and when the Z attached to R² is C, R² is selected from: hydrogen, C₁₋₃alkyl optionally substituted with 1-3 fluoro, —O—C₁₋₃alkyl optionally substituted with 1-3 fluoro, hydroxy, chloro, fluoro, bromo and phenyl;
when the Z attached to R³ is N, R³ is oxygen or is absent, and when the Z attached to R³ is C, R³ is selected from: hydrogen, hydroxy, halo, C₁₋₃alkyl where the alkyl is unsubstituted or substituted with 1-6 substituents independently selected from: fluoro, hydroxy and —COR¹¹, —NR¹²R¹², —COR¹¹, —CONR¹²R¹², —NR¹²COR¹³, —OCONR¹²R¹², —NR¹²CONR¹²R¹², —heterocycle, —CN, —NR¹²—SO₂—NR¹²R¹², —NR¹²—SO₂—R¹⁴, —SO₂—NR¹²R¹² and nitro;
when the Z attached to R⁴is N, R⁴ is oxygen or is absent, and when the Z attached to R⁴is C, R⁴ is selected from: hydrogen, C₁₋₃alkyl optionally substituted with 1-3 fluoro, —O—C₁₋₃alkyl optionally substituted with 1-3 fluoro, hydroxy, chloro, fluoro, bromo and phenyl;
R⁵ is selected from: C₁₋₆alkyl where alkyl is unsubstituted or substituted with 1-6 substituents selected from fluoro and hydroxyl, —O—C₁₋₆alkyl where alkyl is unsubstituted or substituted with 1-6 fluoro, —CO—C₁₋₆alkyl where alkyl is unsubstituted or substituted with 1-6 fluoro, —S—C₁₋₆alkyl where alkyl is unsubstituted or substituted with 1-6 fluoro, pyridyl which is unsubstituted or substituted with one or more substituents selected from: halo, trifluoromethyl, C₁₋₄alkyl, and COR¹¹, fluoro, chloro, bromo, —C₄₋₆cycloalkyl, —O—C₄₋₆cycloalkyl, phenyl which is unsubstituted or substituted with one or more substituents selected from halo, trifluoromethyl, C₁₋₄alkyl, and COR¹¹, —O-phenyl which is unsubstituted or substituted with one or more substituents selected from: halo, trifluoromethyl, C₁₋₄alkyl, and COR¹¹, —C₃₋₆cycloalkyl where alkyl is unsubstituted or substituted with 1-6 fluoro, —O—C₃₋₆cycloalkyl where alkyl is unsubstituted or substituted with 1-6 fluoro, -heterocycle, —CN and —COR¹¹;
when the Z attached to R⁶ is N, R⁶ is oxygen or is absent, and when the Z attached to R⁶ is C, R⁶ is selected from: hydrogen, C₁₋₃alkyl optionally substituted with 1-3 fluoro, —O—C₁₋₃alkyl optionally substituted with 1-3 fluoro, hydroxy, chloro, fluoro, bromo and phenyl;
R⁷ is selected from: hydrogen, C₁₋₈alkyl which is unsubstituted or substituted with 1-6 substituents selected from: hydroxy, halo, —O—C₁₋₆alkyl, CN, —NR¹²R¹², —NR¹²COR¹³, —NR¹²SO₂R¹⁴, —COR¹¹, —CONR¹²R¹², phenyl and heterocycle, where the alkyl, phenyl, and heterocycle are unsubstituted or substituted with 1-3 substituents selected from: halo, hydroxy, C₁₋₃alkyl, C₁₋₃alkoxy, —CO₂H, —CO₂—C₁₋₆alkyl, and trifluoromethyl, and —SO₂C₁₋₆alkyl which is unsubstituted or substituted with 1-6 substituents selected from: hydroxy, halo, —O—C₁₋₆alkyl, CN, —NR¹²R¹², —NR¹²COR¹³, —NR¹²SO₂R¹⁴, —COR¹¹, —CONR¹²R¹², phenyl and heterocycle, where the alkyl, phenyl, and heterocycle are unsubstituted or substituted with 1-3 substituents selected from: halo, hydroxy, C₁₋₃alkyl, C₁₋₃alkoxy, —CO₂H, —CO₂—C₁₋₆alkyl, and trifluoromethyl; R⁸ is selected from C₁₋₁₀alkyl, —SO₂C₁₋₁₀alkyl, pyridyl or phenyl, unsubstituted or substituted with 1-5 substituents selected from: hydroxy, halo, —O—C₁₋₆alkyl, —S—C₁₋₆alkyl, CN, —NR¹²R¹², —NR¹²COR¹³, —NR¹²SO₂R¹⁴, —COR¹¹, —CONR¹²R¹², —SO₂R¹⁴, heterocycle, ═O (where the oxygen is connected via a double bond), phenoxy and phenyl, where the alkyl, phenyl, phenoxy and heterocycle are unsubstituted or substituted with 1-3 substituents selected from: halo, hydroxy, C₁₋₃alkyl, C₁₋₃alkoxy, —COR¹¹, —CN, —NR¹²R¹², —SO₂R¹⁴, —NR¹²COR¹³, —NR¹²SO₂R¹⁴, and —CONR¹²R¹², where the alkyl and alkoxy are optionally substituted with 1-5 fluoro;
R¹⁰ and R¹⁶ are independently selected from: ═O, hydrogen, phenyl, C₁₋₆alkyl which is unsubstituted or substituted with 1-6 of the following substituents: —COR¹¹, hydroxy, fluoro, chloro, and —O—C₁₋₃alkyl; and,
R¹¹ is independently selected from: hydroxy, hydrogen, C₁₋₆ alkyl, —O—C₁₋₆alkyl, benzyl, phenyl, C₃₋₆ cycloalkyl, where the alkyl, phenyl, benzyl, and cycloalkyl groups are unsubstituted or substituted with 1-3 substituents independently selected from: halo, hydroxy, C₁₋₃alkyl, C₁₋₃alkoxy, —CO₂H, —CO₂—C₁₋₆ alkyl, and trifluoromethyl,
R¹² is selected from: hydrogen, C₁₋₆ alkyl, benzyl, phenyl, C₃₋₆ cycloalkyl, where the alkyl, phenyl, benzyl, and cycloalkyl groups are unsubstituted or substituted with 1-3 substituents independently selected from: halo, hydroxy, C₁₋₃alkyl, C₁₋₃alkoxy, —CO₂H, —CO₂—C₁₋₆alkyl, and trifluoromethyl, and
R¹³ is selected from: hydrogen, C₁₋₆ alkyl, —O—C₁₋₆alkyl, benzyl, phenyl, C₃₋₆ cycloalkyl, where the alkyl, phenyl, benzyl, and cycloalkyl groups are unsubstituted or substituted with 1-3 substituents independently selected from: halo, hydroxy, C₁₋₃alkyl, C₁₋₃alkoxy, —CO₂H, —CO₂—C₁₋₆alkyl, and trifluoromethyl,
R¹⁴ is selected from: hydroxy, C₁₋₆ alkyl, —O—C₁₋₆alkyl, benzyl, phenyl, C₃₋₆ cycloalkyl, where the alkyl, phenyl, benzyl, and cycloalkyl groups are unsubstituted or substituted with 1-3 substituents independently selected from: halo, hydroxy, C₁₋₃alkyl, C₁₋₃alkoxy, —CO₂H, —CO₂—C₁₋₆alkyl, and trifluoromethyl,
R¹⁵ is selected from hydrogen and C₁₋₃alkyl;
or, R² and R¹⁵ are joined together to form a carbocycle or heterocycle ring with a linker selected from: —CH₂(CR¹⁷R¹⁷)₁₋₃—, —CH₂NR¹⁸—, —NR¹⁸—CR¹⁷R¹⁷—, —CR¹⁷R¹⁷O—, —CR¹⁷R¹⁷SO₂—, —CR¹⁷R¹⁷SO—, —CR¹⁷R¹⁷S—, —CR¹⁷R¹⁷—, and —NR¹⁸— (with the left side of the linker being bonded to the amide nitrogen at R¹⁵),
R¹⁷ is selected from: hydrogen, hydroxy, halo and C₁₋₃alkyl, where the alkyl is unsubstituted or substituted with 1-6 substituents independently selected from: fluoro, and hydroxy, —NR¹²R¹², —COR¹¹, —CONR¹²R¹², —NR¹²COR¹³, —OCONR¹²R¹², —NR¹²CONR¹²R¹², -heterocycle, —CN, —NR¹²—SO₂—NR¹²R¹², —NR¹²—SO₂—R¹⁴, —SO₂—NR¹²R¹², and ═O, and where when one R¹⁷ is connected to the ring via a double bond the other R¹⁷ at the same position is absent,
R¹⁸ is selected from: hydrogen, C₁₋₃alkyl unsubstituted or substituted with 1-6 substituents independently selected from: fluoro, and hydroxy, COR¹³, SO₂R¹⁴, and SO₂NR¹²R¹²;
the dashed line represents an optional bond;
and pharmaceutically acceptable salts thereof and individual diastereomers thereof.

2. The compound of claim 1 of the formula Ia: 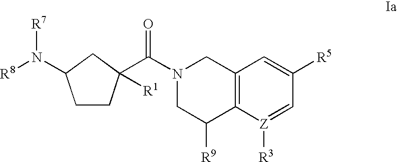
wherein R⁹ is selected from: hydrogen, hydroxy, C₁₋₃alkyl unsubstituted or substituted with 1-6 substituents independently selected from fluoro and hydroxy, —COR¹¹, —CONR¹²R¹², —NR¹²COR¹¹, —NR¹²—SO₂—R¹⁴, —SO₂—NR¹²R¹², and ═O, where R⁹ is connected to the ring via a double bond,
and pharmaceutically acceptable salts thereof and individual diastereomers thereof.

3. The compound of claim 1 of the formula Ib: 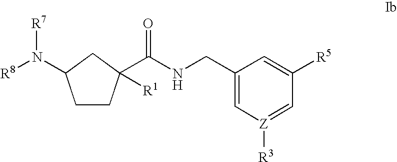
and pharmaceutically acceptable salts thereof and individual diastereomers thereof.

4. The compound of claim 1 of the formula Ic: 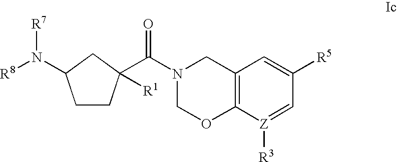
and pharmaceutically acceptable salts thereof and individual diastereomers thereof.

5. The compound of claim 1 of the formula Id: 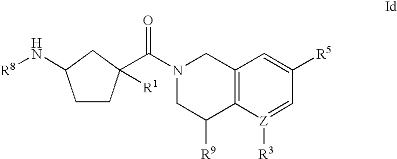
and pharmaceutically acceptable salts thereof and individual diastereomers thereof.

6. The compound of claim 1 of the formula Ie: 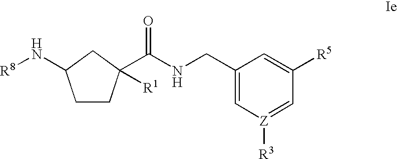
and pharmaceutically acceptable salts thereof and individual diastereomers thereof.

7. The compound of claim 1 of the formula If: 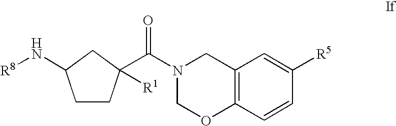
and pharmaceutically acceptable salts thereof and individual diastereomers thereof.

8. The compound of claim 1 wherein R¹ is selected from:
—C₁₋₆alkyl, —C₀₋₆alkyl-O—C₁₋₆alkyl, and —(C₀₋₆alkyl)-(C₃₋₇cycloalkyl)-(C₀₋₆alkyl), where the alkyl and the cycloalkyl are unsubstituted or substituted with 1-7 substituents independently selected from: halo, hydroxy, —O—C₁₋₃alkyl, trifluoromethyl, C₁₋₃alkyl, —O—C₁₋₃alkyl, —COR¹¹, —CN, —NR¹²R¹², and —CONR¹²R¹²,
and pharmaceutically acceptable salts thereof and individual diastereomers thereof.

9. The compound of claim 1 wherein R¹ is selected from:
—C₁₋₆alkyl unsubstituted or substituted with 1-6 substituents independently selected from: halo, hydroxy, —O—C₁₋₃alkyl, trifluoromethyl, and —COR¹¹,
—C₀₋₆alkyl-O—C₁₋₆alkyl-unsubstituted or substituted with 1-6 substituents independently selected from: halo, trifluoromethyl, and —COR¹¹,
—(C₃₋₅cycloalkyl)-(C₀₋₆alkyl) unsubstituted or substituted with 1-7 substituents independently selected from: halo, hydroxy, —O—C₁₋₃alkyl, trifluoromethyl, and —COR¹¹,
and pharmaceutically acceptable salts thereof and individual diastereomers thereof.

10. The compound of claim 1 wherein R¹ is C₁₋₆alkyl unsubstituted or substituted with 1-6 substituents selected from hydroxyl and fluoro, and pharmaceutically acceptable salts thereof and individual diastereomers thereof.

11. The compound of claim 1 wherein R¹ is selected from: —CH(CH₃)₂, —CH(OH)CH₃ and —CH₂CF₃, and pharmaceutically acceptable salts thereof and individual diastereomers thereof.

12. The compound of claim 1 wherein R¹ is selected from: thiazolyl, unsubstituted or substituted with NHCOR¹⁵, and pharmaceutically acceptable salts thereof and individual diastereomers thereof.

13. The compound of claim 1 wherein the Z attached to R² is C, and pharmaceutically acceptable salts thereof and individual diastereomers thereof.

14. The compound of claim 1 wherein R² is hydrogen or R² and R¹⁵ are linked by —CH₂—CH₂— or —CH₂—O—, and pharmaceutically acceptable salts thereof and individual diastereomers thereof.

15. The compound of claim 1 wherein when the Z attached to R³ is N, R³ is absent or is or O, and pharmaceutically acceptable salts thereof and individual diastereomers thereof.

16. The compound of claim 1 wherein when the Z attached to R³is N, R³ is absent, and pharmaceutically acceptable salts thereof and individual diastereomers thereof.

17. The compound of claim 1 wherein when the Z attached to R³ is C, R³ is selected from: hydrogen, halo, hydroxy, C₁₋₃alkyl, where the alkyl is unsubstituted or substituted with 1-6 substituents independently selected from: fluoro, and hydroxy, —COR¹¹, —CONR¹²R¹², -heterocycle, —NR¹²—SO₂—NR¹²R¹², —NR¹²—SO₂—R¹⁴, —SO₂—NR¹²R¹², -nitro, and —NR¹²R¹²;
and pharmaceutically acceptable salts thereof and individual diastereomers thereof.

18. The compound of claim 1 wherein when the Z attached to R³ is C R³ is hydrogen, and pharmaceutically acceptable salts thereof and individual diastereomers thereof.

19. The compound of claim 1 wherein the Z attached to R⁴ is C, and pharmaceutically acceptable salts thereof and individual diastereomers thereof.

20. The compound of claim 1 wherein R⁴ is hydrogen, and pharmaceutically acceptable salts thereof and individual diastereomers thereof.

21. The compound of claim 1 wherein R⁵ is selected from: C₁₋₆alkyl substituted with 1-6 fluoro, —O—C₁₋₆alkyl substituted with 1-6 fluoro, chloro, bromo, and phenyl, and pharmaceutically acceptable salts thereof and individual diastereomers thereof.

22. The compound of claim 1 wherein R⁵ is selected from: trifluoromethyl, trifluoromethoxy, chloro, bromo, and phenyl, and pharmaceutically acceptable salts thereof and individual diastereomers thereof.

23. The compound of claim 1 wherein R⁵ is trifluoromethyl, and pharmaceutically acceptable salts thereof and individual diastereomers thereof.

24. The compound of claim 1 wherein the Z attached to R⁶ is C, and pharmaceutically acceptable salts thereof and individual diastereomers thereof.

25. The compound of claim 1 wherein R⁶ is hydrogen, and pharmaceutically acceptable salts thereof and individual diastereomers thereof.

26. The compound of claim 1 wherein R⁷ is hydrogen or methyl and pharmaceutically acceptable salts thereof and individual diastereomers thereof.

27. The compound of claim 1 wherein R⁷ is hydrogen, and pharmaceutically acceptable salts thereof and individual diastereomers thereof.

28. The compound of claim 1 wherein R⁸ is selected from: C₁₋₈alkyl optionally substituted with hydroxy, C₁₋₆alkyl substituted with 1-6 fluoro, C₁₋₆alkyl substituted with —COR¹¹, benzyl, unsubstituted or substituted with 1-3 substituents selected from: hydroxy, methoxy, chloro, fluoro, —COR¹¹, methyl and trifluoromethyl, —CH₂-pyridyl, unsubstituted or substituted with 1-3 substituents selected from: hydroxy, methoxy, chloro, fluoro, methyl and trifluoromethyl, and pharmaceutically acceptable salts thereof and individual diastereomers thereof.

29. The compound of claim 1 wherein R⁹ is hydroxy, hydrogen, ═O, where R⁹ is connected to the ring via a double bond, and pharmaceutically acceptable salts thereof and individual diastereomers thereof.

30. The compound of claim 1 wherein R⁹ is hydrogen, and pharmaceutically acceptable salts thereof and individual diastereomers thereof.

31. The compound of claim 1 wherein R¹⁰ is hydrogen and pharmaceutically acceptable salts thereof and individual diastereomers thereof.

32. The compound of claim 1 wherein R¹⁵ is hydrogen or is joined to R², and pharmaceutically acceptable salts thereof and individual diastereomers thereof.

33. The compound of claim 1 wherein R¹⁶ is and pharmaceutically acceptable salts thereof and individual diastereomers thereof.

34. A compound selected from: 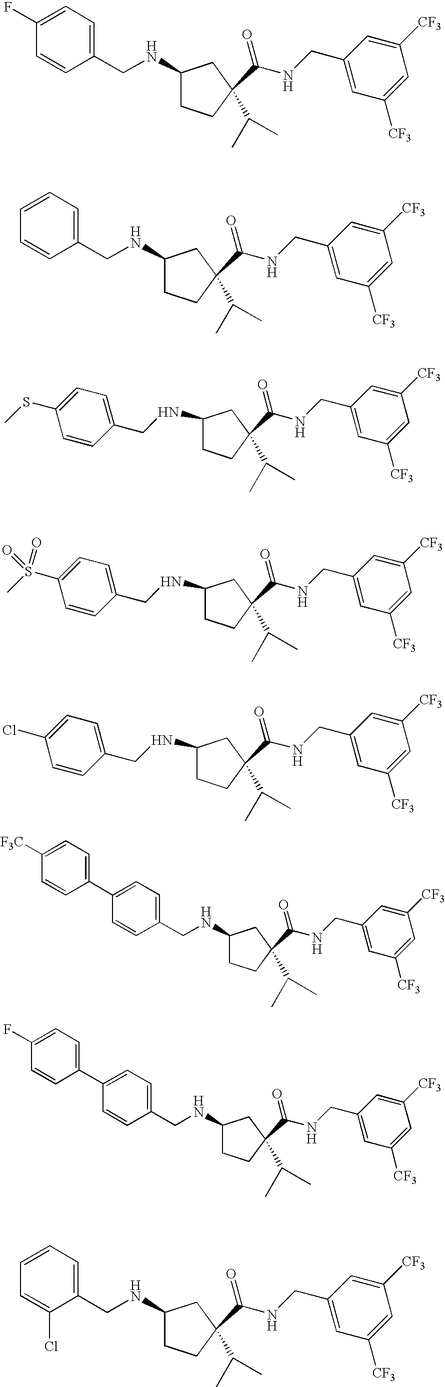 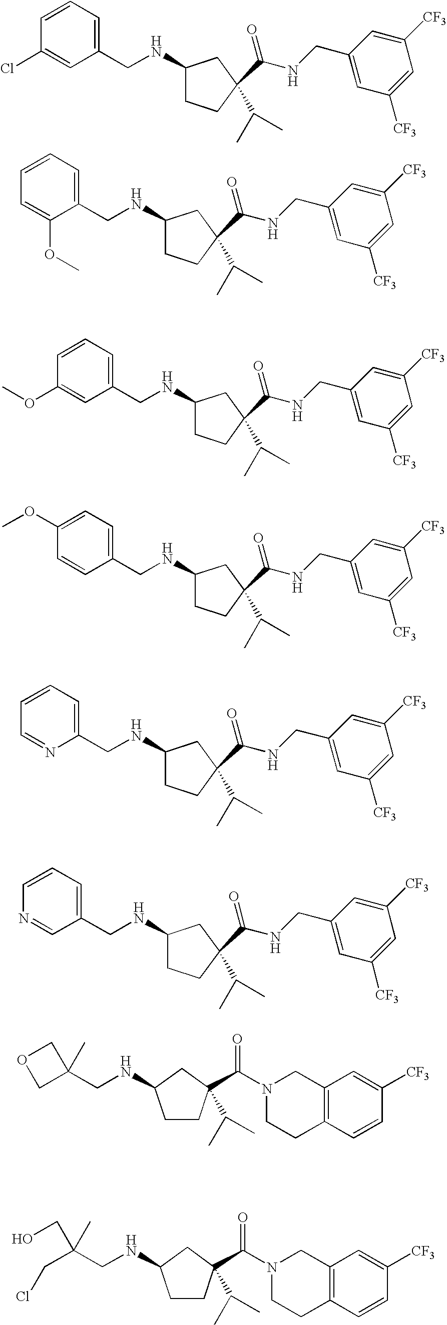 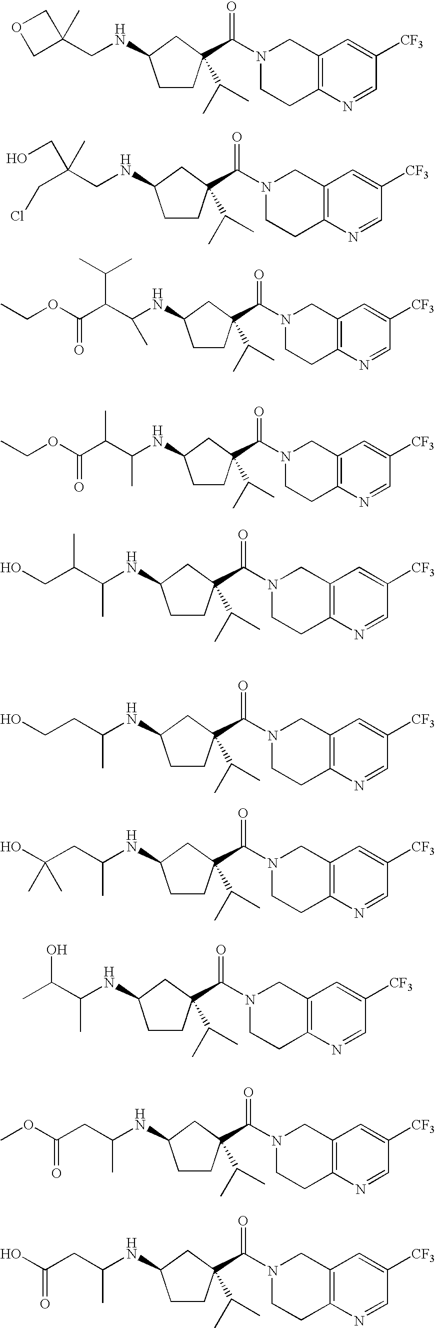 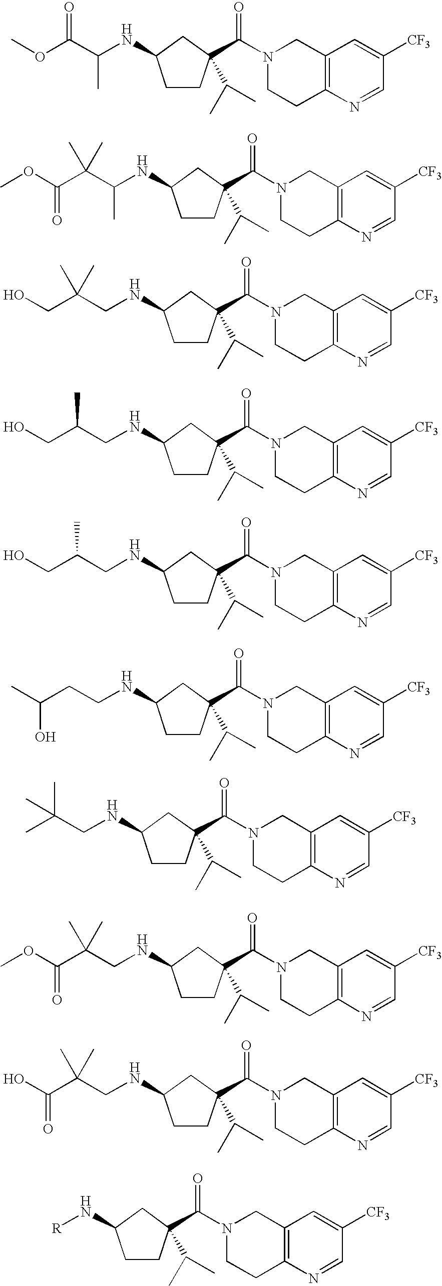 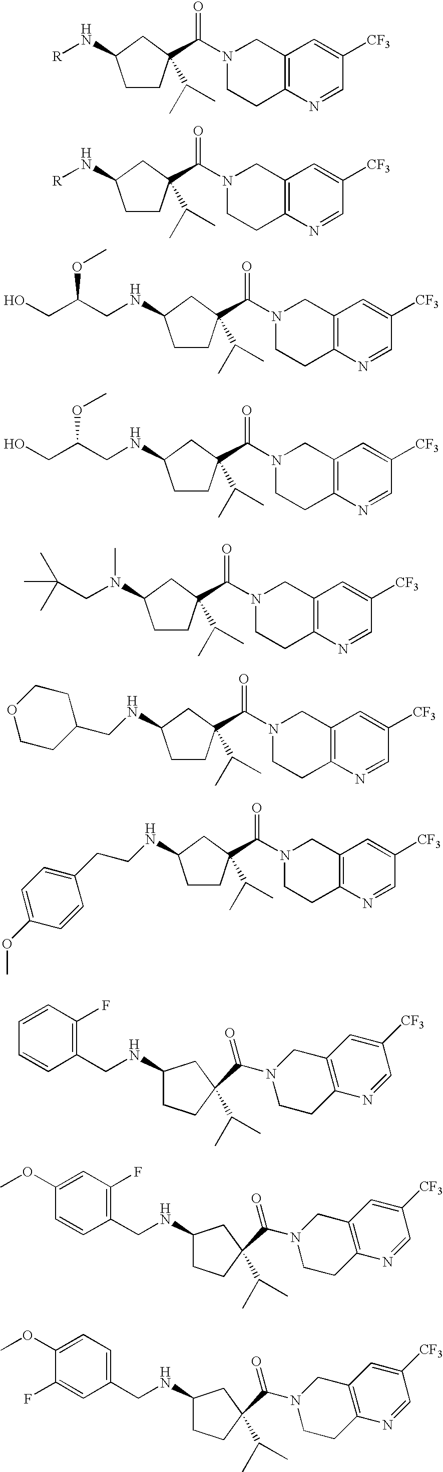 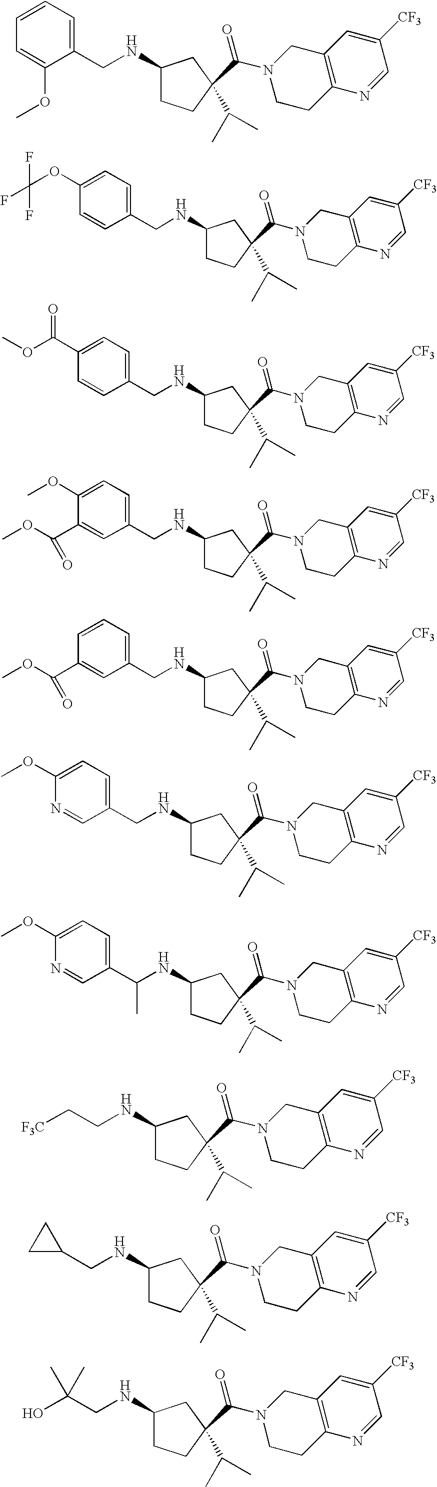 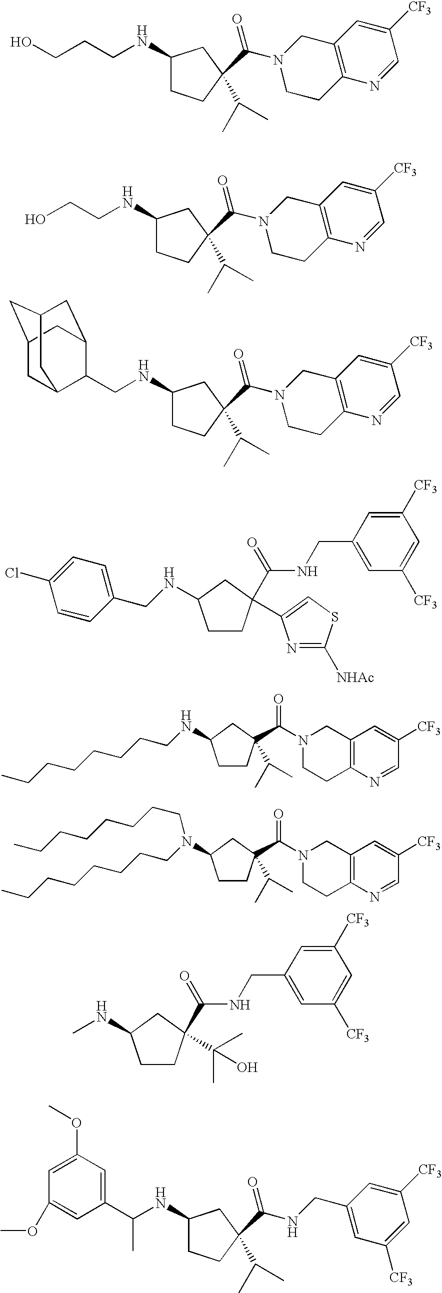 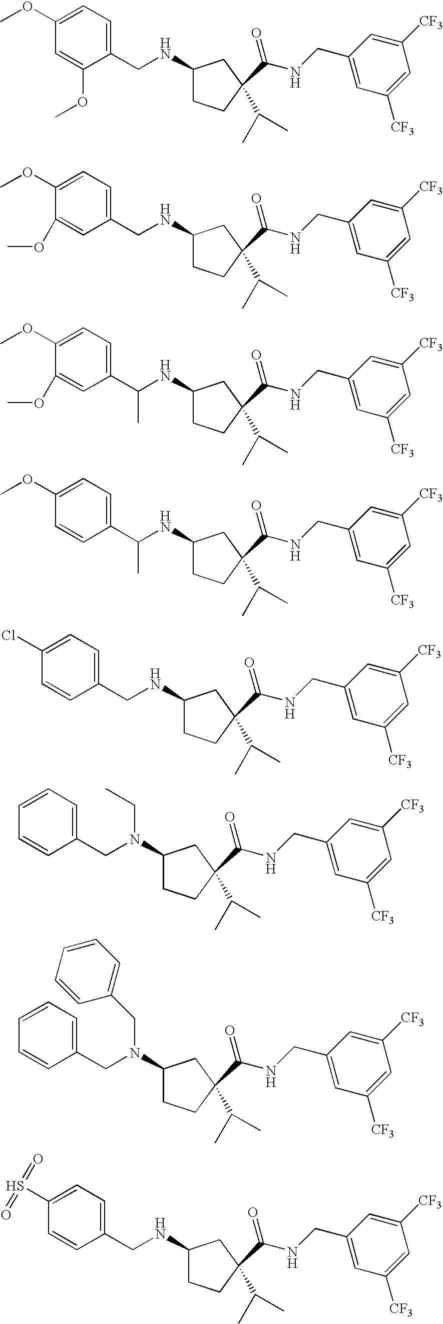 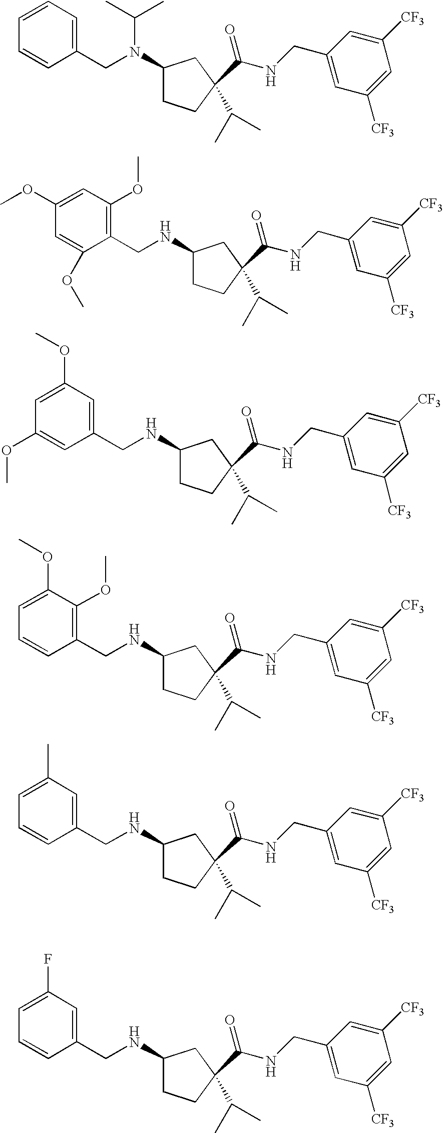 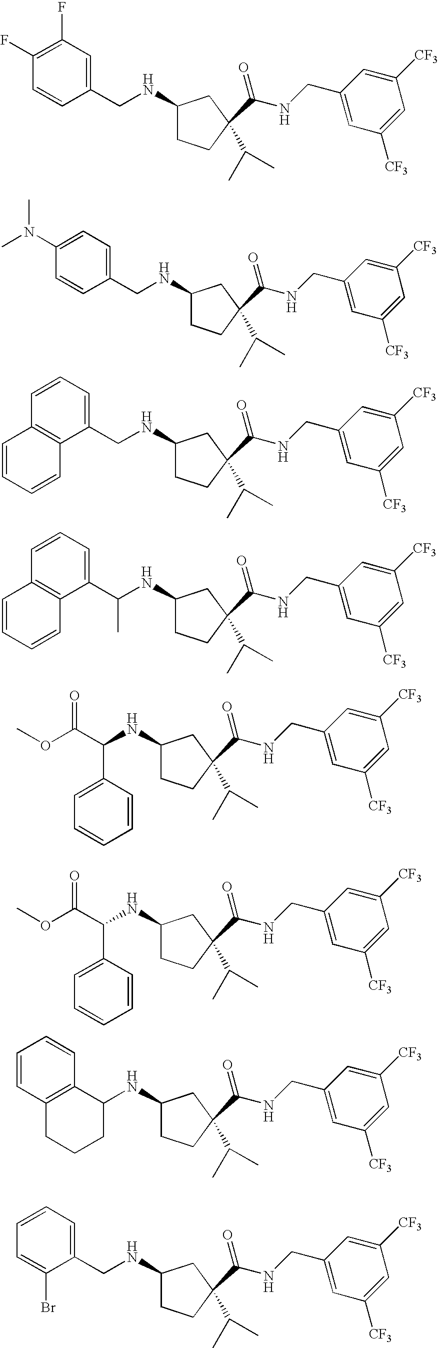 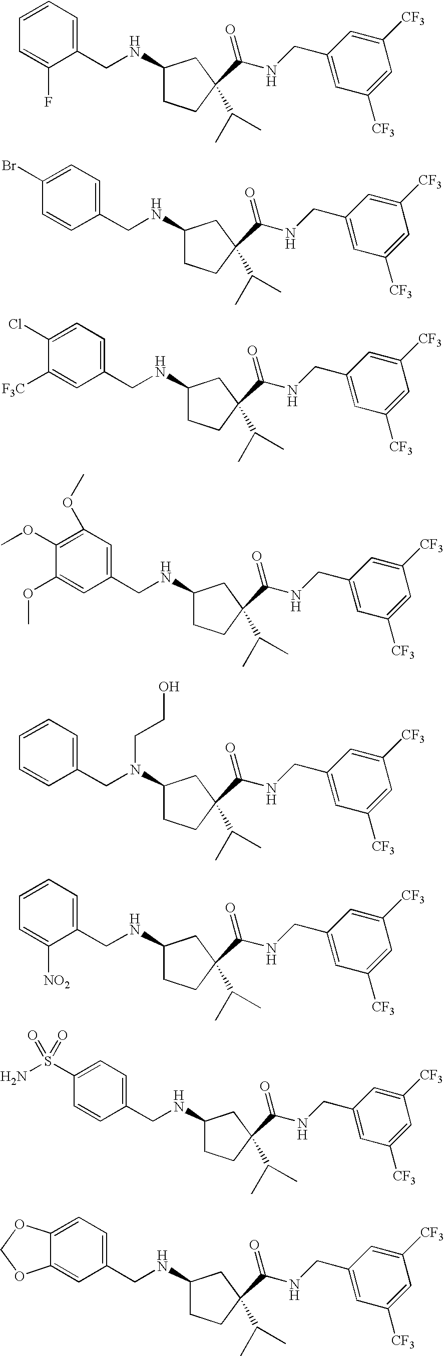 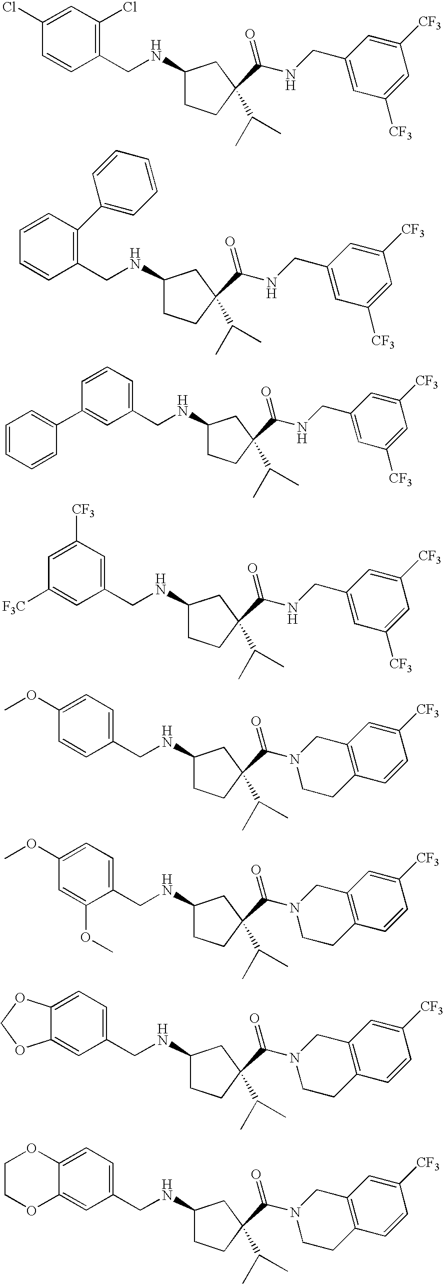 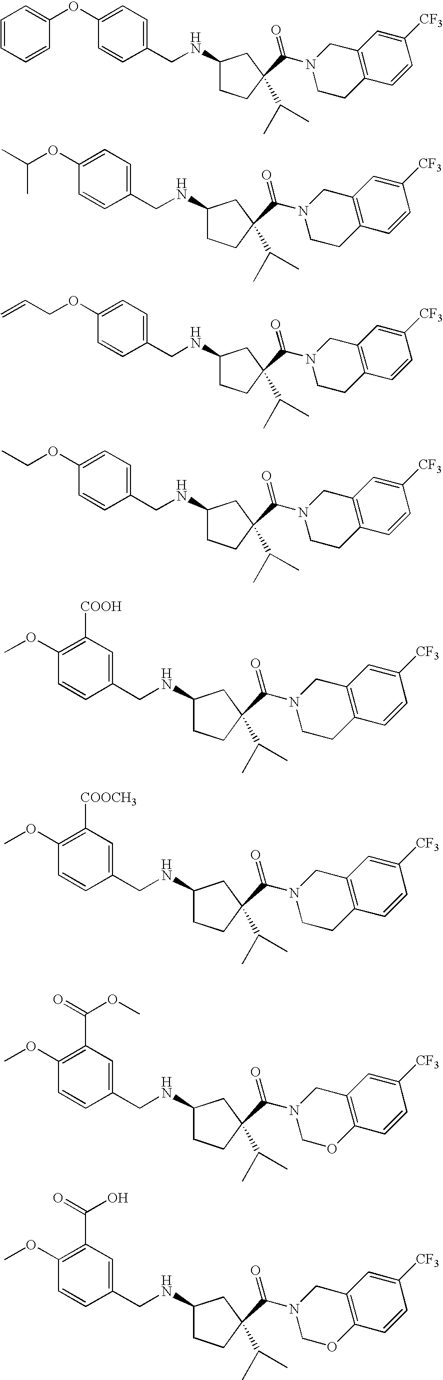 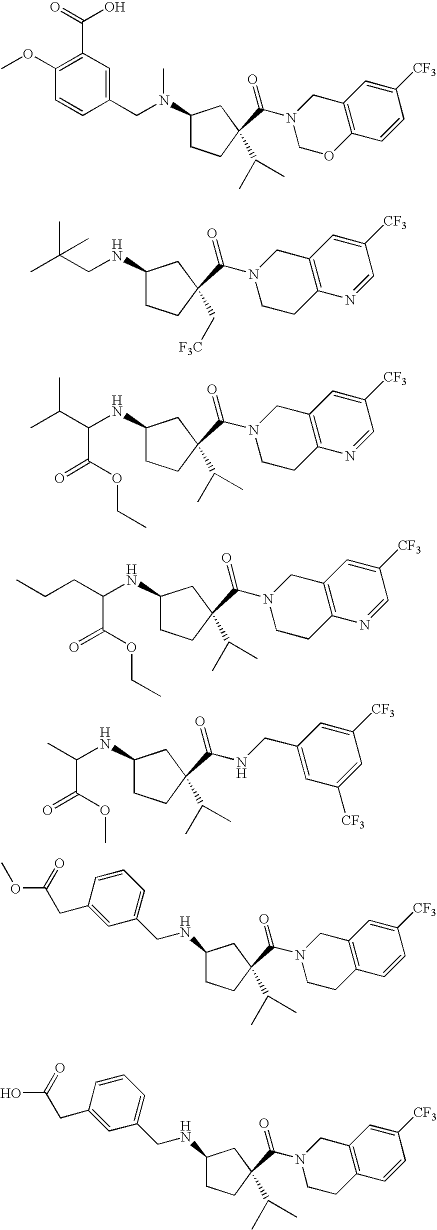
and pharmaceutically acceptable salts thereof and individual diastereomers thereof.

35. A pharmaceutical composition which comprises an inert carrier and a compound of claim 1.

36. A method for modulations of chemokine receptor activity in a mammal which comprises the administration of an effective amount of a compound of claim 1.

37. A method for treating, ameliorating, controlling or reducing the risk of an inflammatory and immunoregulatory disorder or disease which comprises the administration to a patient of an effective amount of a compound of claim 1.

38. A method for treating, ameliorating, controlling or reducing the risk of rheumatoid arthritis which comprises the administration to a patient of an effective amount of a compound of claim 1.
